(19) United States
(12) Patent Application Publication
(10) Pub. No.: US 2013/0183309 A1
(43) Pub. Date: Jul. 18, 2013

(54) Fragments, Mutants and Chimeric Fusion Proteins of Leptin For Treating Alzheimer's Disease

(71) Applicant: Nikolaos Tezapsidis, West Orange, NJ (US)

(72) Inventor: Nikolaos Tezapsidis, West Orange, NJ (US)

(21) Appl. No.: 13/799,932

(22) Filed: Mar. 13, 2013

Related U.S. Application Data
(63) Continuation-in-part of application No. 13/439,804, filed on Apr. 4, 2012.
Application No. 13/439,804 is a continuation of application No. 11/516,224, filed on Sep. 6, 2006, now Pat. No. 8,227,408.

(51) Int. Cl. A61K 38/22; A61K 45/06

(52) U.S. Cl. CPC A61K 38/2264; A61K 45/06
USPC 424/134.1; 514/5.8

(57) ABSTRACT

The described invention relates to methods for treating, preventing, or diagnosing the pathology of progressive cognitive disorders resulting from accumulation of an amyloid peptide, in particular, Alzheimer's disease, Down's syndrome and cerebral amyloid angiopathy, in mammalian subjects using a composition comprising therapeutically effective amount of a leptin, leptin mimic, leptin derivative, leptin agonist, or AMP-dependent protein kinase activator, alone, or in combination with, one or more lipolytic/antilipogenic compounds. It further relates to methods for improving cognitive function using a composition comprising a therapeutically effective amount of leptin, a leptin mimic, a leptin derivative, an AMP-dependent protein kinase activator, a leptin agonist, a leptin blocker, a mimic of a leptin blocker, a leptin antagonist, an AMP-dependent protein kinase inhibitor; or a pharmaceutically acceptable salt thereof.

### CROSS-REFERENCE TO RELATED APPLICATION

This application is a continuation-in-part of U.S. application Ser. No. 13/439,804, filed Apr. 4, 2012, which is a continuation of U.S. application Ser. No. 11/516,224, filed Sep. 6, 2006, issued as U.S. Pat. No. 8,227,408, which claims the benefit of priority to U.S. Provisional Application No. 60/714,948, filed Sep. 7, 2005. The entire content of each of these applications is hereby incorporated by reference in its entirety.

### FIELD OF THE INVENTION

The present invention relates to methods for treating, preventing, or diagnosing the pathology of progressive cognitive disorders resulting from accumulation of an amyloid peptide or tau hyperphosphorylation.

### BACKGROUND OF THE INVENTION

Weight loss frequently is observed in Alzheimer's disease (AD) patients prior to the onset of dementia, supportive of an underlying metabolic disorder. (Barrett-Connor et al., J Am Geriatr Soc. 44:1147-52 (1996); Bissoli et al., J Nutr Health Aging. 6:247-53 (2002)). Furthermore, lipid homeostasis (meaning the multi-layered regulatory networks of lipid metabolism, transport, and signal transduction) specifically, as exemplified in cell culture and animal models in addition to clinical studies with lipid-lowering agents, e.g., statins, can have an impact on amyloidogenic pathways. Such pathways lead to the generation of amyloid β (Aβ) peptide through proteolytic processing of the amyloid precursor protein (APP). (Stefan F. Lichtenthaler and Christian Haass, I Clin. Invest. 113:1384-1387 (2004); Puglielli et al., Nature Cell Biol. 3:905-912. (2001)). An important modulator of lipid homeostasis in non-adipose tissues is the pluripotent peptide leptin (Unger in Annu Rev Med. Vol. 53. 319-36 (2002).

Many neurons in the brain regions typically affected in AD (entorrhinal cortex, hippocampus, parahippocampus gyrus, amygdale, frontal, temporal, parietal and occipital association cortices, and certain subcortical nuclei projecting to these regions) contain large, nonmembrane-bound bundles of abnormal fibers that occupy much of the perinuclear cytoplasm. Most of these fibers consist of pairs of 10 nm filaments wound into helices (paired helical filaments (PHF)), with a helical period of about 160 nm. Some tangle-bearing neurons also contain skeins of straight, 10 nm to 15 nm filaments interspersed with the PHF. Neurofibrillary tangles (NFTs) are aggregates of the microtubule-associated protein “tau”, which have become hyperphosphorylated and accumulate inside the cells themselves. Tau is relatively abundant in neurons but is present in all nucleated cells and functions physiologically to bind microtubules and to stabilize microtubule assembly for polymerization. During neurodegeneration, tau is phosphorylated abnormally at proline directed serine/threonine phosphorylation sites, which can be detected using specific antisera. These serine/threonine (Ser/Thr) phosphorylation sites include Ser-202/Thr-205 (AT8 site), Ser-214 and/or Ser-214, Ser-181, and/or Ser-212 (AT1OO site), Thr-231 and/or Ser-235 (TG3 site), and Ser-396/Ser-404 (PHF-I site).

The correlation between regional distribution of phosphorylated tau and clinical signs suggests a close relationship between tau and AD pathogenesis. The increased tau phosphorylation that accompanies AD may result in separation of tau from the microtubule, possibly aided by other factors (such as, for example, Aβ, oxidative stress, inflammatory mediators), and sequestration of NFTs and neuropil threads. Without being limited by theory, the loss of normal tau function (stabilization and maintenance of microtubules), combined with a toxic gain of function, could compromise axonal transport and contribute to synaptic degeneration. The role of NFT toxicity, however, remains unclear. Studies have indicated that mice models expressing a repressible human tau still developed NFTs, neuronal loss, and behavioral impairments; after tau suppression, the behavioral deficits stabilized, yet NFTs continued to accumulate. In another AD-like model, axonal pathology with accumulation of tau proceeded plaque deposition. NFTs (and presumable “intermediates”) exist within the cytoplasm of viable neurons. Only in advanced disease are large numbers of extracellular NFTs identified.

NFTs are not specific for AD, particularly if a broader definition of NFTs includes different tau isoforms or if one expands the expectation of the morphological characteristics of NFTs. The two classical lesions of AD, neuritic plaques and NFTs, can occur independently of each other. Tangles composed of tau aggregates that are biochemically similar to, or in some cases, indistinguishable from those in AD have been described in more than a dozen less common neurodegenerative diseases, in almost all of which one finds no Aβ deposits and neuritic plaques.

In addition to deregulation of lipid metabolism in the CNS, the immune system has been implicated in the pathobiology of Alzheimer's disease. Amyloid plaques are decorated with proteins of the complement system, eicosanoids and cytokines, integral components of ongoing inflammatory processes that augment the harmful effects of NI (Emmerling et al., Biochim Biophys Acta 1502: 158-71 (2000)). Important regulators of the immune system include the cytokines and chemokines secreted by leukocytes (B or T cells, normally scarce in the brain) or antigen presenting cells (APC) (microglia, perivascular macrophages, astrocytes in the brain). In AD brain, both pro-inflammatory cytokines and anti-inflammatory cytokines are expressed (Benveniste et al., Neurochem Intl, 39: 381-91 (2001)). In addition to immune function, cytokines may directly affect the processing of APP (Blasko et al., FASEB J. 13: 63-68 (1999)). Leptin has very similar structural and functional characteristics to the cytokines (Heshka, J. T., and P. J. Jones, Life Sci. 69:987-1003 (2001)), sharing post-receptor pathways and participating in our immune response to pathogens and infections. Leptin deficiency is associated with impaired T cell immunity (Faggioni, R., K. R. Feingold, and C. Grunfeld. 2001. FASEB J. 15:2565-71 (2001)) and increased sensitivity to the lethal effects of bacterial endotoxin and TNF-a. Most importantly, these effects can be reversed with leptin administration, which attenuates inflammatory cytokine and neuroendocrine responses to infection (Xiao et al., Endocrinology 144: 4350-53 (2003)). Further, in critically ill septic patients, higher leptin levels are positively correlated with survival (Arnalich et al., J. Infect. Dis. 180: 908-11 (1999)).

Leptin is a peptide hormone that controls adaptive metabolic mechanisms to energy availability leading to storage or mobilization of fat (Schwartz et al., Nature. 404: 661-71 (2000)). Adipocyte-derived leptin primarily exerts its central action through the arcuate nucleus neurons (an aggregation of neurons in the mediobasal hypothalamus); however, it can affect other populations, including hippocampal neurons and cells of the periphery (Shanley et al., Nat Neurosci. 5:299-300 (2002)). Ablation of leptin or of leptin signaling is sufficient to cause obesity as exemplified by leptin-deficient obese, hyperinsulinemic mice having the genotype ob/ob; diabetic mice with a mutation in the leptin receptor gene having the genotype db/db, which produce but are non-responsive to leptin; rats of the genotype fa/fa, which have the “fatty” obesity gene, which is a mutated leptin receptor; and in a few rare genetic cases (Schwartz et al., Nature. 404: 661-71 (2000)).

Leptin has been investigated as a novel therapy for obesity. While the peptide was well tolerated by humans and exhibited an excellent safety profile in clinical trials, it failed to demonstrate efficacy for the targeted condition.

The primary amino acid sequence of Leptin indicated that it adopts a 4-helix bundle structure similar to that of the cytokine IL-2, which has since been verified by x-ray crystallography (Zhang, F. et al., Nature 387 (1997) 206-209). Recent structural studies have modeled the Leptin-Leptin Receptor (LR) complex as a hexamer. In this model, each Leptin molecule interacts with three different LR chains through domains I, II and III of Leptin (FIG. 8). These domains are similar to the long-chain cytokine IL-6 which interacts with its receptor through three different binding domains I-III (Peelman, F. et al., J. Biol. Chem. 279 (2004) 41038-41046; Peelman, F. et al., J. Biol. Chem. 281 (2006) 15496-15504). Leptin contains all three LR-interacting domains at both its N- and C-termini as well as a single disulfide bond at its C-terminus, resulting in formation of a C-terminal loop at positions 117-167 (FIG. 8).

The structure-function relationship of domains II and III of Leptin are well described, however less is known about the role of domain I (Table 2). Domain II is responsible for binding of the Leptin molecule to the LR signaling chain, with limited effect on receptor activation, while domain III is involved in signaling, with limited effect on receptor binding (Peelman, F. et al., J. Biol. Chem. 279 (2004) 41038-41046). Domain I incurs a modest effect on receptor activation, but is hypothesized to function in binding to the non-signaling α-chain of the LR (Peelman, F. et al., J. Biol. Chem. 279 (2004) 41038-41046).

TABLE 2
Leptin Structure and Functional Domains

| | Secondary | Primary | |
|---|---|---|---|
| Domain | Structure | Sequence | Function |
| I | A-B Loop | 62 | Binding to non-signaling chain |
| | | | of OB-R? |
| | C-terminus | 155-159 | Modest effect on receptor activa- |
| | Helix D | | tion |
| II | Helix A | 30-41 | Binding to signaling chain of |
| | | | OB-R |
| | Helix C | 96-113 | Limited effect on receptor |
| | | | activation |
| III | N-terminus A-B | 50-55 | No effect on receptor binding |
| | Loop | | |
| | Helix E | 129-143 | Receptor activation/signaling |
| | C-terminus C-D | | |
| | Loop | | |
| | N-terminus | | |
| | Helix D | | |

Initial studies of Leptin's structure-function relationship came from analysis of the Leptin mutant found in the ob/ob mouse (Zhang, Y. et al., Nature 372 (1994) 425-432), which contains a false stop codon at position 105. This truncated Leptin molecule is 83 amino acids (aa) long (after cleavage of the 21 aa signal sequence, see FIG. 8) and lacks the C-terminal loop. The absence of truncated Leptin in the plasma of these mice indicated that the truncated molecule either was unstable or failed to be secreted from adipocytes. These studies supported a role for the C-terminal loop in Leptin's bioactivity. Likewise, patients with the extremely rare disorder human congenital Leptin deficiency, share an obese phenotype and low to undetectable levels of Leptin in the plasma as observed for the ob/ob mouse (Montague, C. T. et al., Nature 387 (1997) 903-908). However, these patients possess a frameshift mutation at position 133 which results in the incorrect addition of 14 aa and a false stop codon. The resulting 126 aa truncated Leptin molecule lacks a disulfide bond. These findings suggest the importance of the disulfide bond in producing a functional Leptin molecule. Other studies have shown the necessity of the C-terminal loop in BBB transport as well as effects on Leptin stability, solubility and secretion (Imagawa, K. et al., J. Biol. Chem. 273 (1998) 35245-35249; Giese, K. et al., Molecular Medicine 2 (1996) 50-58; Muller, G. et al., J. Biol. Chem. 272 (1997) 10585-10593). In contrast, a 35 aa N-terminal fragment of Leptin (22-56) (Samson, W. K. et al., Endocrinology 137 (1996) 5182-5185) as well as N-terminal fragments lacking either the C-terminal loop or a disulfide-bond (Imagawa, K. et al., J. Biol. Chem. 273 (1998) 35245-35249) have shown biological activity when injected into the brains of rats and mice, respectively.

The leptin receptor (ObR), a member of the class I cytokine receptor superfamily (Lord, G. M., et al. Nature 394:897 (1998)) has at least six isoforms as a result of alternative splicing. As used herein the term “isoform” refers to a version of a protein that has the same function as another version of the protein but that has some small differences in its sequence. All isoforms of ObR share an identical extracellular ligand-binding domain (Couce et al., Neuroendocrinology. 66:145-50 (1997)). Leptin's functional receptor (ObRb), the b isoform, is expressed not only in the hypothalamus, where it regulates energy homeostasis and neuroendocrine function, but also in other brain regions and in the periphery, including all cell types of innate and adaptive immunity (Lord, G. M., et al., Nature 394:897 (1998); Zhao, Y., R. et al., Biochem. Biophys. Res. Commun. 300: 247 (2003)); Zarkesh-Esfahani, H., G. et al., J. Immunol. 157: 4593 (2001) CaldefieChezet, F., A. et al., J. Leukocyte Biol. 69:414 (2001)). The full-length b isoform (ObRb) lacks intrinsic tyrosine kinase activity and is involved in several downstream signal transduction pathways.

Leptin binding to its functional receptor recruits Janus tyrosine kinases and activates the receptor, which then serves as a docking site for cytoplasmic adaptors such as STAT (Baumann, H., et al. Proc. Natl. Acad. Sci. USA 93:8374 1996)). According to the general model for JAK/STAT activation, STAT proteins initially are present in inactive forms in the cytoplasm Following ligand stimulation and receptor dimerization, the JA1C/STAT pathway is activated by activation of receptor-bound JAK kinases. These JAK kinases subsequently phosphorylate the receptor at tyrosine residues, which recruits STATs to the receptor. STATs then are phosphorylated to form phosphoSTATs, dimerized, and translocated to the nucleus, where the phosphoSTAT dimers bind to specific sequences in the promoter regions of their target genes, and stimulate the transcription of these genes (Schindler et al., Ann. Rev. Biochem. 64: 621-51 (1995)), including negative regulators, such as the suppressor of cytokine signaling 3 (Bjorbaek, C., K. et al. J. Biol. Chem. 274:30059 (1999)) and the protein tyrosine phosphatase 1B (Cheng, A. N. et al. Dev. Cell 2:497 (2002), Schwartz et al., Nature, 404:661-71 (2000), Louis A. Tartaglia, J. Biol. Chem. Minireview, 272:6093-6096 (March 1997)).

In addition to the JAK-2-STAT-3 pathway, other pathways also are involved in mediating leptin's effect in the brain and on the immune cells. For example, the mitogen-activated protein kinase (MAPK) pathways, the insulin receptor substrate 1 (IRS1) pathway, and the phosphatidylinositol 3′-kinase (MK) pathway (Martin-Romero, C., V. Sanchez-Margalet. Cell. Immunol. 212:83 (2001)) also mediate leptin's action (Sanchez-Margalet, V., C. Martin-Romero, Cell. Immunol. 211:30 (2001)).

Leptin may have a physiologic role as a liporegulatory hormone responsible for maintaining intracellular homeostasis in the face of wide variations in caloric intake (Unger R H. 2003. Annu Rev Physiol. 65:333-47). This is achieved by directly stimulating lipolysis, (meaning fat breakdown), and inhibiting lipogenesis (meaning fat synthesis) (Lee Y, et al., J. Biol Chem. 276(8):5629-35 (2001)). Leptin can improve insulin resistance and hyperglycemia by a mechanism not completely understood (Toyoshima et al., Endocrinology 146: 4024-35 (2005)), despite insulin's ability to stimulate lipogenesis (Kersten, EMBO Reports 2(4): 282-286 (2001). Both insulin and Aβ are degraded by insulin degrading enzyme (IDE).

The levels of cholesterol and fatty acids in cells also are regulated tightly by a single family of transcription factors named Sterol Regulatory Element-Binding Proteins (SREBPs) which activate relevant target genes (Brown and Goldstein, Cell. 89:331-40 (1997)). SREBPs are transcription factors that regulate the expression of genes for both cholesterol and fatty acid synthesis. The inactive precursor form of SREBPs resides in cytoplasmic membranes. Intracellular lipid depletion triggers proteolytic cleavage of the SREBPs, allowing the amino terminus to enter the nucleus and activate the expression of enzymes, including acetyl-CoA carboxylase (ACC) and fatty acid synthase (FAS), major biosynthetic enzymes for fatty acid synthesis. (Wilentz, Robb E. et al., Pediatric and Developmental Pathology, 3 (6): 525-531 (2000)).

In the central nervous system (CNS, meaning the brain and spinal cord), metabolic pathways involving lipids serve mainly to provide the building blocks for membranes, vitamins, second messengers and to modify proteins by acylation, because there are no main mechanisms for utilizing triglycerides/fatty acids as energy sources.

It is well documented that brain lipids are intricately involved in Amyloid β (Aβ)-related pathogenic pathways. The Aβ peptide is the major proteinaceous component of the amyloid plaques found in the brains of Alzheimer's disease (AD) patients and is regarded by many as the culprit of the disorder. The amount of extracellular Aβ accrued is critical for the pathobiology of AD and clearly depends on the antagonizing rates of its production/secretion and its clearance. It has been shown (Tezapsidis et al., FASEB 1 17:1322-1324 (2003)) that neurons depend on the interaction between Presenilin 1 (PS1) and Cytoplasmic-Linker Protein 170 (CLIP-170) to both generate Aβ and to take it up through the lipoprotein receptor related protein (LRP) pathway. Further to this requirement, formation of Aβ depends on the assembly of key proteins in lipid rafts (LRs) (Simons et al., Proc Natl Acad Sci USA. 95: 6460-4 (1998)). The term “lipid rafts” as used herein refers to membrane microdomains enriched in cholesterol, glycosphingolipids and glucosylphosphatidyl-inositol-(GPI)-tagged proteins implicated in signal transduction, protein trafficking and proteolysis. Within the LRs it is believed that Aβ's precursor, Amyloid Precursor Protein (APP), a type I membrane protein, is cleaved first by the protease β-secretase (BACE) to generate the C-terminal intermediate fragment of APP, CAPPβ, which remains imbedded in the membrane. The amino acid sequence of Aβ peptide showing its cleavage sites and membrane domain is shown in FIG. 1 a. CAPPβ is subsequently cleaved at a site residing within the lipid bilayer by y-secretase, a high molecular weight multi-protein complex containing presenilin, (PS1/PS2), nicastrin, PEN-2, and APH-1 or fragments thereof (De Strooper, Neuron. 38: 9-12 (2003)). Aβ finally is released outside the cell, where it can: a) start accumulating following oligomerization and exerting toxicity to neurons or b) be removed either by mechanisms of endocytosis (involving apolipoprotein-E (apoE) and LRP or Scavenger Receptors) or by degradation by extracellular proteases including insulin-degrading enzyme (IDE) and neprilysin (Farris et al., Proc Natl Acad Sci USA. 100:4162-4167 (2003)) (FIG. 1 b).

Fatty acid and cholesterol availability and cellular composition modifies the transbilayer distribution of cholesterol and, consequently, overall membrane fluidity, function and localization of lipid rafts, a process which changes with aging (Wood et al., Neurobiol Aging. 23:685-694 (2002)). Therefore, it was hypothesized that leptin's lipolytic/antilipogenic activity could affect the composition of the LRs, affecting Aβ turnover.

U.S. Pat. No. 8,227,408 describes leptin's ability to modify the levels of Aβ both in vitro and in vivo. Leptin, similarly to methyl-β-cyclodextrin, reduces β-secretase activity in neuronal cells, possibly, but without being limited by theory, by altering the lipid composition of membrane LRs. This contrasts the results of treatments with cholesterol and etomoxir (an inhibitor of carnitine-palmitoyl transferase-1). Conversely, inhibitors of acetyl CoA carboxylase and fatty acid synthase mimicked leptin's action. Additionally, leptin was able to increase apoEdependent Aβ uptake in vitro. Thus, leptin can modulate indirectly bi-directional Aβ kinesis, reducing its levels extracellularly. Chronic administration of leptin to AD-transgenic animals reduced the brain Aβ load, illustrating its therapeutic potential.

The described invention discloses that fragments of leptins, derivatives of leptins, mutants of leptins, and chimeric fusion proteins of leptins share these properties.

### SUMMARY OF THE INVENTION

According to one aspect, the described invention provides a method for treating a progressive cognitive disease, cognitive disorder, or cognitive condition resulting from accumulation of an amyloid peptide, comprising: administering to a subject in need thereof a first composition comprising: (i) a therapeutic amount of a leptin, a leptin mimic, or a pharmaceutically acceptable salt thereof, and (ii) a pharmaceutically acceptable carrier, wherein the leptin or the leptin mimic is a recombinant human leptin, a pegylated recombinant human leptin (PEG-OB), a recombinant human methionyl leptin, a leptin peptidomimetic, a biologically active fragment of leptin, a fusion peptide of leptin with an Fc fragment of immunoglobulin, a fusion peptide of the biologically-active fragment of leptin with the Fc fragment of immunoglobulin, a leptin agonist, or a combination thereof, wherein the therapeutic amount of the leptin or the leptin mimic is effective to modulate accumulation of the amyloid peptide in brain. According to one embodiment, the method further comprises monitoring circulating levels of the amyloid peptide. According to another embodiment, the circulating levels of amyloid peptide are detected in a sample of cerebrospinal fluid or blood. According to another embodiment, the method further comprises placing the subject on a low fat diet. According to another embodiment, the progressive cognitive disease, cognitive disorder, or cognitive condition is a dementia, an amyloidosis, Down's syndrome, or cerebral amyloid angiopathy. According to another embodiment, the progressive cognitive disease, cognitive disorder, or cognitive condition is Alzheimer's disease. According to another embodiment, the progressive cognitive disease, cognitive disorder, or cognitive condition is senile systemic amyloidosis. According to another embodiment, the progressive cognitive disease, cognitive disorder, or cognitive condition is cerebrovascular amyloidosis. According to another embodiment, the amyloid peptide is an amyloid β (Aβ) peptide. According to another embodiment, the first composition further comprises (iii) a therapeutically effective amount of one or more lipolytic/antilipogenic compounds wherein the one or more lipolytic/antilipogenic compounds reduce amyloid β (Aβ) production, increase apoE-Aβ (Aβ) uptake, or both. According to another embodiment, the first composition modulates accumulation of the amyloid peptide in the cerebral nervous system. According to another embodiment, the first composition is administered by at least one route selected from the group consisting of orally, buccally, parenterally, intranasally, rectally, and topically. According to another embodiment, the method further comprises serially administering a second composition comprising a therapeutically effective amount of one or more lipolytic/antilipogenic compounds, wherein the one or more lipolytic/antilipogenic compounds reduce amyloid β (Aβ) production, increase apoE-Aβ (Aβ) uptake, or both. According to another embodiment, the method further comprises placing the subject on a low fat diet.

According to another aspect, the described invention provides a method of improving resilience of cognitive function in a subject in need thereof, the method comprising: (a) administering to the subject a composition comprising: i. a cognitive function-enhancing or cognitive function stabilizing amount of leptin, a leptin mimic, or a pharmaceutically acceptable salt thereof, wherein the leptin or the leptin mimic is a recombinant human leptin, a pegylated recombinant human leptin (PEG-OB), a recombinant human methionyl leptin, a leptin peptidomimetic, a biologically-active fragment of leptin, a fusion peptide of leptin with an Fc fragment of immunoglobulin, a fusion peptide of the biologically-active fragment of leptin with the Fc fragment of immunoglobulin, a leptin agonist, and a combination thereof, and ii. a pharmaceutically acceptable carrier, wherein the cognitive function-enhancing or the cognitive function stabilizing amount of leptin or the leptin mimic is effective to modulate accumulation of an amyloid peptide in brain. According to one embodiment, the composition is administered orally, buccally, parenterally, intranasally, rectally, virally or topically. According to another embodiment, the method further comprises (b) measuring the subject's ability to perform mental tasks. According to another embodiment, the subject's ability to perform mental tasks is measured by at least one test for memory, computation, or attention. According to another embodiment, the biologically active fragment of leptin comprises an amino acid sequence selected from the group consisting of SEQ ID NOs: 27, 28, 29, 30, 31, 32, 33, 34, 35, 36, 37, 38, 39, 40, 43, 44 and 45. According to another embodiment, the biologically active fragment of leptin comprises a first and a second fragment, wherein the first fragment has amino acid sequence SEQ ID NO: 41, wherein the second fragment has amino acid sequence SEQ ID NO: 42, and wherein the first fragment is covalently linked to the second fragment via a disulfide bond between cysteine at amino acid residue 96 of SEQ ID NO: 41 and cysteine at amino acid residue 8 of SEQ ID NO: 42. According to another embodiment, the therapeutic amount is from about 0.01 mg per kg (of body weight) per day to about 0.5 mg per kg (of body weight) per day. According to another embodiment, the subject in need thereof has a systemic leptin deficiency. According to another embodiment, the composition restores, replenishes, or increases leptin levels. According to another embodiment, the biologically active fragment of leptin has an amino acid sequence selected from the group consisting of SEQ ID NOs: 27, 28, 29, 30, 31, 32, 33, 34, 35, 36, 37, 38, 39, 40, 43, 44 and 45. According to another embodiment, the biologically active fragment of leptin comprises a first and a second fragment, wherein the first fragment has amino acid sequence SEQ ID NO: 41, wherein the second fragment has amino acid sequence SEQ ID NO: 42, and wherein the first fragment is covalently linked to the second fragment via a disulfide bond between cysteine at amino acid residue 96 of SEQ ID NO: 41 and cysteine at amino acid residue 8 of SEQ ID NO: 42. According to another embodiment, the cognitive function-enhancing amount of the leptin, the leptin mimic, or the pharmaceutically acceptable salt thereof, reduces amyloid β (Aβ) production, increases apoE-Abeta (Aβ) uptake, or both. According to another embodiment, the amyloid peptide is an amyloid β (Aβ) peptide. According to another embodiment, the subject in need thereof has a systemic leptin deficiency. According to another embodiment, the composition restores, replenishes, or increases leptin levels. According to another embodiment, the cognitive function-enhancing amount is from about 0.01 mg per kg (of body weight) per day to about 0.5 mg per kg (of body weight) per day. According to another embodiment, the composition further comprises (iii) a therapeutically effective amount of one or more lipolytic/antilipogenic compounds, wherein the one or more lipolytic/antilipogenic compounds reduce amyloid β (Aβ) production, increase apoE-Aβ (Aβ) uptake, or both. According to another embodiment, the method further comprises placing the subject on a low fat diet. According to another embodiment, the method further comprises serially administering a second composition comprising a therapeutically effective amount of one or more lipolytic/antilipogenic compounds, wherein the one or more lipolytic/antilipogenic compounds reduce amyloid β (Aβ) production, increase apoE-Aβ (Aβ) uptake, or both.

According to another aspect, the described invention provides a method for treating a progressive cognitive disease, cognitive disorder, or cognitive condition resulting from an increase in tau phosphorylation, comprising: administering to a subject in need thereof a first composition comprising (i) a therapeutic amount of a leptin, a leptin mimic, or a pharmaceutically acceptable salt thereof, and (ii) a pharmaceutically acceptable carrier, wherein the leptin or the leptin mimic is a recombinant human leptin, a pegylated recombinant human leptin (PEG-OB), a recombinant human methionyl leptin, a leptin peptidomimetic, a biologically active fragment of leptin, a fusion peptide of leptin with an Fc fragment of immunoglobulin, a fusion peptide of the biologically-active fragment of leptin with the Fc fragment of immunoglobulin, a leptin agonist, or a combination thereof, wherein the therapeutic amount of the leptin or the leptin mimic is effective to decrease tau phosphorylation in brain. According to one embodiment, the progressive cognitive disease, cognitive disorder, or cognitive condition is a dementia, an amyloidosis, Down's syndrome, or cerebral amyloid angiopathy. According to another embodiment, the progressive cognitive disease, cognitive disorder, or cognitive condition is Alzheimer's disease. According to another embodiment, the progressive cognitive disease, cognitive disorder, or cognitive condition is senile systemic amyloidosis. According to another embodiment, the progressive cognitive disease, cognitive disorder, or cognitive condition is cerebrovascular amyloidosis. According to another embodiment, the composition is administered orally, buccally, parenterally, intranasally, rectally, or topically. According to another embodiment, the biologically active fragment of leptin comprises an amino acid sequence selected from the group consisting of SEQ ID NOs: 27, 28, 29, 30, 31, 32, 33, 34, 35, 36, 37, 38, 39, 40, 43, 44 and 45.

According to another aspect, the described invention provides a method for increasing neuronal cell viability in a subject suffering from a progressive cognitive disease, cognitive disorder, or cognitive condition comprising: administering to a subject in need thereof a first composition comprising (i) a therapeutic amount of a leptin, a leptin mimic, or a pharmaceutically acceptable salt thereof, and (ii) a pharmaceutically acceptable carrier, wherein the leptin or the leptin mimic is a recombinant human leptin, a pegylated recombinant human leptin (PEG-OB), a recombinant human methionyl leptin, a leptin peptidomimetic, a biologically active fragment of leptin, a fusion peptide of leptin with an Fc fragment of immunoglobulin, a fusion peptide of the biologically-active fragment of leptin with the Fc fragment of immunoglobulin, a leptin agonist, or a combination thereof, wherein the therapeutic amount of the leptin or the leptin mimic is effective to increase neuronal cell viability in the subject. According to one embodiment, the progressive cognitive disease, cognitive disorder, or cognitive condition is a dementia, an amyloidosis, Down's syndrome, or cerebral amyloid angiopathy. According to another embodiment, the progressive cognitive disease, cognitive disorder, or cognitive condition is Alzheimer's disease. According to another embodiment is senile systemic amyloidosis. According to another embodiment, the progressive cognitive disease, cognitive disorder, or cognitive condition is cerebrovascular amyloidosis. According to another embodiment, the composition is administered orally, buccally, parenterally, intranasally, rectally, or topically. According to another embodiment, the biologically active fragment of leptin comprises an amino acid sequence selected from the group consisting of SEQ ID NOs: 27, 28, 29, 30, 31, 32, 33, 34, 35, 36, 37, 38, 39, 40, 43, 44 and 45.

### BRIEF DESCRIPTION OF THE DRAWINGS

FIG. 1a shows the amino acid sequence, cleavage sites and membrane domain of Aβ.

FIG. 1b shows mechanisms of Aβ production and clearance.

FIG. 2 shows pathways related to or affected by leptin, leading to inhibition of lipogenesis and stimulation of lipolysis, inhibiting Aβ production.

FIG. 3 shows the amyloidogenic and anti-amyloidogenic pathways (from Lichtenthaler, S. F. and Haass, C., J. Clin. Invest. 113:1384-1387 (2004)).

FIG. 4 shows that leptin affects Aβ production through BACE in rafts. Asterisks indicate that the value is significantly different from that of the corresponding control (set at p<0.05).

In panel (a), Neuro2a cells stably transfected with hyg-sal 34 were treated for about 2 h or about 5 h with about 100 ng/ml leptin, Ob (black); about 125 mg/ml cyclodextrin, CDX (gray stripe); about 5 mg/ml cholesterol, Ch (pale gray); leptin plus cholesterol, Ob+Ch (medium gray).

In panel (b), Neuro2a cells stably transfected with hyg-sal34 were treated for about 2 h or 5 h with about 400 ng/ml leptin, Ob (black); about 250 mg/ml cyclodextrin, CDX (gray stripe), 10 mg/ml cholesterol, Ch (pale gray) and leptin plus cholesterol, Ob+Ch (medium gray).

In panel (c), extracts of SY5Y cells treated with about 400 ng/ml leptin, about 10 μg/ml cholesterol, or both about 400 ng/nl leptin and about 10 μg/ml cholesterol, in the presence of the γ-secretase inhibitors L-685,458 (100 nM) or Z-VL-CHO (100 μM) for about 5 h analysed by SDS-PAGE and Western blotting using an antibody directed against the C-terminal fraction of APP (C-APP, lanes 1-4), actin (top lanes 5-8) or full-length APP (bottom lanes 5-8).

In panel (d), SDS-PAGE and Western blot analysis of sucrose gradient fractions of Triton-X solubilized extracts prepared from SY5Y cells treated with about 400 ng/ml leptin, about 10 μg/ml cholesterol, or both about 400 ng/nl leptin and about 10 μg/ml cholesterol, in the presence of the γ-secretase inhibitors L-685,458 (100 nM) or Z-VL-CHO (100 pM) for about 5 h to detect APP and flotillin (a marker for lipid rafts).

In panel (e), sucrose gradient fractions in (d) were assayed for β-secretase activity using a fluorescence-quenching assay (QTL Biosystems, NM).

FIG. 5 shows that leptin affects apoE-dependent Aβ-uptake and the possible involvement of SREBPs.

In panel (a), Aβ uptake in SY5Y cells following treatment with about 0 ng/ml, about 100 ng/ml or about 400 ng/ml leptin. Uptake did not take place without apoE (white). Uptake was also dependent on PS1 and LRP, as shown in cells previously transfected with antisense DNA for PS1 (black) and in cells treated with RAP (gray stripe) respectively. Leptin induced a dose-dependent increase in Ai3 uptake with a preference for apoE3 (medium gray) over apoE4 (light gray).

In panel (b), Aβ uptake in SY5Y cells pre-treated with about 10 mg/ml cholesterol (+Chol) or normal medium (−Chol). in the absence (black) or the presence (gray) of about 400 ng/ml leptin is shown. Cells were more resistant to taking-up Aβ when pre-loaded with cholesterol. Asterisks indicate that the value is significantly different to that set as 100% (set at p<0.05).

In panel (c), measurement of Aβ in the medium of SY5Y cells transiently transfected with SREBP-1 cDNA, SREBP-2 cDNA, or an empty vector (Control) by ELISA following treatment with (+) or without (−) leptin is shown.

In panel (d), measurement of Aβ uptake in SY5Y cells transiently transfected with transcriptionally active forms of SREBP-1 a cDNA, SREBP-2 cDNA, or an empty vector (Control) following treatment with (+) or without (−) leptin is shown.

FIG. 6 shows that leptin modulates free cholesterol-rich membrane domains and that surplus cholesterol may trigger leptin. Neural cultures from E15 rat cerebral cortex were processed for enrichment in neurons (a-d) or astrocytes (e-h) and, after about 7 days to 10 days in culture, treated for about 5 h with about 10 μg/ml cholesterol (b, f) or about 400 ng/ml leptin plus cholesterol (c, g) or leptin alone (d, h). Controls (a, e) were treated with media alone. Cells were stained for filipin. Neurons (i-k) and astrocytes (l-n) treated with about 0 μM (i, I), about 5 μM (j, m) or about 10 μM cholesterol (k, n) for about 5 h were immunostained for leptin.

FIG. 7 shows the deficiency of leptin in AD transgenic mice and the effect of leptin supplementation on amyloid load.

In panel (a), plasma leptin was quantified in one year old mice with the following genotypes: a) double mutant APP_{swe}/PS1_{M146V} b) single mutant PS1_{M146V} and c) wild-type (a cross between C57BL/6Ntac and B6SJLF1). Asterisk indicates that the value is significantly different to that of non-transgenic controls (set at p<0.05).

In Panel (b) Tg2576 mice under a high fat diets (HFD) and a low fat diets (LFD) from one week prior to the implantation of the Alzet pump subcutaneously (s.c) for constant delivery of leptin (+) or vehicle PBS (−). Pump was replaced after 4 weeks for another 4 week period of treatments. Aβ40 and Aβ42 content in formic acid brain extracts prepared from Tg2576 and wild type (WT) mice were determined by ELISA. Plasma total AP (Aβ40 plus Aβ42/43) was measured in 10 month Tg2576 mice following a 2 month LFD or HFD with (+) or without (−) leptin infusion.

In panel (c), plasma leptin levels were determined by RIA in 10 month old Tg2576 and WT littermate mice following treatments as described in FIG. 7b. Leptin also was measured in WT (but not Tg2576) mice prior to treatment.

In panel (d), plasma insulin levels were determined by RIA in 8 month old WT and Tg2576 mice and then again following a 2 month LFD or HFD with (+) or without (−) leptin infusion.

In panel (e), plasma total Aβ (Aβ40 plus Aβ42/43) was measured in 8 month Tg2576 mice and then again following a 2 month LFD or HFD with (+) or without (−) leptin infusion.

FIG. 8 shows a graphical representation of the leptin molecule's structure and functional domains.

FIG. 9 shows the effect of full length leptin and leptin fragments on RA-SY5Y cell viability. Panel A shows the effect of full length leptin and leptin fragments on RA-SY5Y cell viability in the absence of cholesterol. Panel B shows the effect of full length leptin and leptin fragments on RA-SY5Y cell viability in the presence of cholesterol.

FIG. 10 shows the effect of full length leptin and leptin fragments on tau phosphorylation in RA-SY5Y cells. Panel A shows the effect of full length leptin and leptin fragments on tau phosphorylation in RA-SY5Y cells in the absence of linoleic acid. Panel B shows the effect of full length leptin and leptin fragments on tau phosphorylation in RA-SY5Y cells in the presence of linoleic acid.

FIG. 11 shows the effect of full length leptin and leptin fragments on Aβ production in RA-SY5Y cells. Panel A shows the effect of full length leptin and leptin fragments on Aβ production in RA-SY5Y cells in the absence of ceramide. Panel B shows the effect of full length leptin and leptin fragments on Aβ production in RA-SY5Y cells in the presence of ceramide.

### DETAILED DESCRIPTION OF THE INVENTION

The present invention can be better understood from the following description of exemplary embodiments, taken in conjunction with the accompanying figures and drawings. It should be apparent to those skilled in the art that the described embodiments of the present invention provided herein are merely exemplary and illustrative and not limiting.

Alzheimer's disease (AD) is characterized histologically by the presence of extracellular amyloid deposits in the brain, together with widespread neuronal loss. Extracellular amyloid deposits are known as neuritic or senile plaques. Amyloid deposits can also be found within and around blood vessels. The main protein constituent of AD and AD-like senile plaques, a peptide known as Aβ, is a normal proteolytic product of a much larger transmembrane protein, the amyloid precursor protein (APP). Aβ can be detected in plasma and cerebrospinal fluid (CSF) in vivo, and in cell culture media in vitro. The terms “amyloid peptide” “amyloid 0 peptide” and “Aβ” are used interchangeably herein to refer to the family of peptides generated through proteolytic processing of the amyloid precursor protein (APP). APP exists as three different spliced isoforms, one having 770 amino acids (isoform a) (SEQ ID NO:1), one having 751 amino acids (isoform b) (SEQ ID NO:2), and one having 695 amino acids (SEQ ID NO:3). The term “APP” as used herein refers to all three isoforms. The terms “amyloid peptide” “amyloid 0 peptide” and “Aβ” include, but are not limited to, Aβ40 (SEQ ID NO:4), Aβ42 (SEQ ID NO:5) and Aβ43 (SEQ ID NO:6). The two major forms of Aβ are Aβ40 (SEQ ID NO:4), corresponding to a 40 amino acid-long peptide and Aβ42 (SEQ ID NO:5), corresponding to a 42 amino acid-long peptide. Aβ43 (SEQ ID NO:6) corresponds to a 43 amino acid-long Aβ peptide.

The term “administer”, “administering” or “to administer” as used herein, refers to the giving or supplying of a medication, including in vivo administration, as well as administration directly to tissue ex vivo. Generally, compositions may be administered systemically either orally, bucally, parenterally, topically, by inhalation or insufflation (i.e., through the mouth or through the nose) or rectally in dosage unit formulations containing conventional nontoxic pharmaceutically acceptable carriers, adjuvants and vehicles as desired, or may be locally administered by means such as, but not limited to, injection, implantation, grafting, topical application or parenterally.

The terms “agent” and “therapeutic agent” are used interchangeably herein to refer to a drug, molecule, composition, or other substance that provides a therapeutic effect. The term “active agent” as used herein, refers to the ingredient, component or constituent of the compositions of the present invention responsible for the intended therapeutic effect.

The terms “amino acid residue” or “amino acid” or “residue” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids, including amino acids with secondary modifications such as acetylation or other substitutions. The abbreviations used herein for amino acids are those abbreviations which are conventionally used: A=Ala=Alanine; R=Arg=Arginine; N=Asn=Asparagine; D=Asp=Aspartic acid; C=Cys=Cysteine; Q=Gln=Glutamine; E=Glu=Glutamic acid; G=Gly=Glycine; H=His=Histidine; I=Ile=Isoleucine; L=Leu=Leucine; K=Lys=Lysine; M=Met=Methionine; F=Phe=Phenyalanine; P=Pro=Proline; S=Ser=Serine; T=Thr=Threonine; W=Trp=Tryptophan; Y=Tyr=Tyrosine; V=Val=Valine. The amino acids may be L- or D-amino acids. An amino acid may be replaced by a synthetic amino acid which is altered so as to increase the half-life of the peptide or to increase the potency of the peptide, or to increase the bioavailability of the peptide. The following represent groups of amino acids that are conservative substitutions for one another:

### Alanine (A), Serine (S), Threonine (T);

### Aspartic Acid (D), Glutamic Acid (E);

### Asparagine (N), Glutamine (Q);

### Arginine (R), Lysine (K);

### Isoleucine (I), Leucine (L), Methionine (M), Valine (V); and

### Phenylalanine (F), Tyrosine (Y), Tryptophan (W).

The terms “amyloid peptide”, “amyloid B peptide”, and “AB” are used interchangeably herein to refer to the family of peptides generated through proteolytic processing of the amyloid precursor protein (APP).

The term “amyloidoses” as used herein refers to a group of conditions of diverse etiologies characterized by the accumulation of insoluble fibrillar proteins (amyloid) in various organs and tissues of the body, wherein eventually organ function is compromised. The associated disease states may be inflammatory, hereditary or neoplastic and the deposition of the amyloid peptide may be localized, generalized or systemic.

The described invention provides a method for treating or preventing the pathology of a disease, disorder or condition resulting from accumulation of an amyloid peptide in a subject. According to one embodiment,

the amyloid peptide is an amyloid β peptide. Such a disease, disorder or condition may be any cognitive impairment, including, but not limited to, a dementia; amyloidoses, such as AD and senile systemic amyloidosis; Down's syndrome (patients with Down's syndrome, characterized by trisomy 21, have an extra copy of APP and develop senile plaques from about 12 years of age); cerebral amyloid angiopathy (CAA), also known as congophilic angiopathy or cerebrovascular amyloidosis (a disease of small blood vessels in the brain in which deposits of amyloid protein in the vessel walls may lead to stroke, brain hemorrhage, or dementia); as well as diseases, disorders or conditions co-morbid with (meaning occurring in association with) AD or with any of the above diseases, disorders or conditions, such as Parkinson's disease and epilepsy.

The term “cognitive function” refers to the intellectual processes resulting in an understanding, perception, or awareness of one's ideas as well as the ability to perform mental tasks, such as thinking, learning, judging, remembering, computing, controlling motor functions, and the like.

The term “dementia” as used herein refers to a decline or a progressive decline in cognitive function due to damage or disease in the brain beyond what might be expected from normal aging.

As used herein the term “treating” includes abrogating, substantially inhibiting, slowing or reversing the progression of a disease, condition, or disorder, substantially ameliorating clinical or aesthetical symptoms of a condition, substantially preventing the appearance of clinical or aesthetical symptoms of a disease, condition, or disorder, and protecting from harmful or annoying stimuli.

The term “disease” or “disorder” as used herein refers to an impairment of health or a condition of abnormal functioning. The term “syndrome,” as used herein, refers to a pattern of symptoms indicative of some disease or condition. The term “injury,” as used herein, refers to damage or harm to a structure or function of the body caused by an outside agent or force, which may be physical or chemical. The term “condition”, as used herein, refers to a variety of health states and is meant to include disorders or diseases caused by any underlying mechanism or disorder, injury, and the promotion of healthy tissues and organs.

The term “subject” as used herein includes animal species of mammalian origin, including humans. It further includes cells and tissues derived from these species.

According to one embodiment, accumulation of amyloid peptide in the disease, disorder or condition may occur extracellularly, meaning located or occurring outside a cell or cells. In a further embodiment, the accumulation of amyloid peptide is in the central nervous system (CNS) of the subject, and may be either in the brain or on cerebral blood vessels walls.

According to one aspect, the method of the present invention comprises the step of administering to a subject susceptible to or having a disease, disorder or condition resulting from accumulation of an amyloid peptide a composition comprising (i) a therapeutically effective amount of leptin, a leptin mimic, a leptin derivative, or a leptin agonist, and (ii) a pharmaceutically acceptable carrier, and thereby modulating accumulation of the amyloid peptide. As used herein, the term “modulate” or “modulating” refers to adjusting, changing, or manipulating the function or status of amyloid peptide accumulation. Such modulation may be any change in the rate of accumulation, including an undetectable change.

According to another embodiment of the method of the present invention, the method comprises monitoring circulating levels of amyloid peptide. Such monitoring may be performed one or more times at any point, i.e., before, during, or after, administration of leptin to a subject. Methods for monitoring include measuring leptin levels detected in a sample of cerebrospinal fluid or blood collected from the subject.

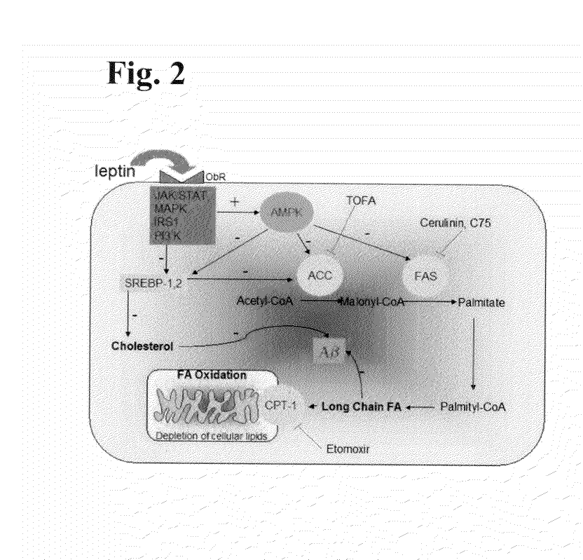

The terms “leptin mimic, leptin mimetic or leptin peptidomimetic” are used interchangeably herein to refer to a leptin derivative comprising a functional domain of the leptin protein, alone or in combination with another molecule, which will produce a biological effect, namely the effect of modulating amyloid peptide levels in a subject. More specifically, a peptidomimetic is a compound containing non-peptidic structural elements capable of mimicking or antagonizing (meaning neutralizing or counteracting) the biological action(s) of a natural parent peptide. Particularly useful for the present invention is a peptidomimetic incorporating the portion of leptin mediating activity, such as decreasing amyloid peptide levels, that is of a size small enough to penetrate the blood-brain barrier. Likewise, a leptin agonist is a compound capable of activating the leptin receptor and/or downstream effectors (see FIG. 2) and modulating amyloid peptide levels in a subject. Moreover, an activator of AMP-dependent protein kinase (AMPK) may have anti-amyloidogenic activity, based on AMPK's ability to promote lipolysis and inhibit lipogenesis upon activation. For example, phenformin and 5-aminoimidazole-4-carboxamide riboside (AICAR) are two drugs widely used to activate AMPK experimentally (King et al. Biochem. Pharmacol. 71:1637-47 (2006)). In addition, the antidiabetic drugs metformin and rosiglitazone may also exert some of their pharmacological actions through AMPK.

The terms “blood brain barrier” or “blood-CSF barrier” are used to describe naturally-occurring systems for excluding substances from the brain and for transporting substances from blood to CSF or brain and vice versa to preserve homeostasis in the nervous system. The barriers facilitate entry of necessary metabolites, but block entry or facilitate removal of unnecessary metabolites or toxic substances. For any solute (i.e., a substance dissolved in and by a solvent), the efficacy of the exclusion or the transport is determined by morphological and functional characteristics of the brain and spinal cord capillaries and by the biochemical and biophysical characteristics of the solute. The barrier systems include carrier-mediated transport systems. Since lipid solubility enhances the transport of substances, ionized polar compounds enter the brain slowly unless there is a specific transport system for them.

Also useful according to the present invention is a leptin blocker; mimic, mimetic or peptidomimetic of a leptin blocker, such as a leptin-binding protein; or a leptin antagonist, which increases amyloid peptide levels. Also, compounds capable of inhibiting AMPK (e.g., compound C) can have leptin blocking properties. For example, and without limitation, such blockers or inhibitors are useful in providing an experimental approach to accelerate AD-like pathology in existing animal models of AD, and for in vitro experimental approaches.

The term “derivative” as used herein refers to an amino acid sequence produced from a leptin-derived peptide, either directly or by modification or partial substitution of the leptin-derived peptide. For example, and without limitation, derivatives of leptin include truncated and fusion leptin products (see infra).

The term “peptide” as used herein, refers to a molecule of two or more amino acid chemically linked together. A peptide may refer to a polypeptide, protein or peptidomimetic. The peptides of the invention may comprise D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Synthetic amino acids include ornithine for lysine, and norleucine for leucine or isoleucine. In addition, the peptides can have peptidomimetic bonds, such as ester bonds, to prepare peptides with novel properties. For example, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a peptide would be resistant to protease activity, and would possess an extended half-live in vivo. Accordingly, these terms also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The term “peptide” is also inclusive of modifications including, but not limited to, glycosylation, lipid attachment, sulfation, gamma-carboxylation of glutamic acid residues, hydroxylation and ADP-ribosylation. In some embodiments, the peptide is of any length or size. Use herein of the terms “peptide”, “peptides”, or “peptidomimetic” should be taken to include reference to “derivatives” of such compounds, unless the context requires otherwise, and to include “prodrugs.”

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity”.

The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information at ncbi.nlm.nih.gov. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA, 1989, 89:10915).

In addition to calculating percent sequence identity, the BLAST algorithm also performs a statistical analysis of the similarity between two sequences (see, e.g., Karlin & Altschul, Proc. Natl. Acad. Sci. USA, 1993, 90: 5873-5787). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wooten and Federhen, Comput. Chem., 1993, 17:149-163) and XNU (Claverie and States, Comput. Chem., 1993, 17:191-201) low-complexity filters may be employed alone or in combination.

As used herein, “sequence identity” or “identity” in the context of two nucleic acid or polypeptide sequences refers to the residues in the two sequences which are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 1988, 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) relative to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid.

The terms “substantial identity” in the context of a peptide indicates that a peptide comprises a sequence with at least 70% sequence identity to a reference sequence, at least 80%, at least 85%, at least 90% or 95% sequence identity to the reference sequence over a specified comparison window. Optionally, optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions that are not identical may differ by conservative amino acid changes.

The administered composition according to the described invention may further comprise a therapeutically effective amount of one or more lipolytic/antilipogenic compounds. The term “lipolytic compound” as used herein refers to a compound whose activity pertains to, is characterized by, or causes lipolysis (meaning the disintegration or splitting of fats). The term “antilipogenic compound” as used herein refers to a compound whose activity pertains to, is characterized by, or causes inhibition of lipid synthesis. In a preferred embodiment, the lipolytic/antilipogenic compound may be an acetyl CoA carboxylase inhibitor (such as 5-(tetraecyloxy)-2-furoic acid (TOFA)), a fatty acid synthase inhibitor (such as cerulenin), an acetyl CoA carboxylase inhibitor and a fatty acid synthase inhibitor, or an AMPK activator. In addition, the administered composition may be used in conjunction with other pharmaceuticals.

Furthermore, if the subject in need of treatment according to the method of the described invention has indications of other complications, such as cardiovascular disease, diabetes, or is a carrier of the apoE [symbol] 4 allele, the subject also may be instructed to follow additional varied treatment regimens. As used herein the term “allele” refers to an alternative DNA coding of the same gene occupying a given gene locus. The [symbol] 4 allele of the apoE gene likely constitutes a major risk factor for amyloid β peptide accumulation and late-onset AD. One such regimen may be to follow a low-fat diet in combination with treatments described herein.

In another aspect, the described invention provides a method of modulating amyloid peptide accumulation in a subject comprising interfering with (meaning affecting or disrupting) at least one step in at least one metabolic or signaling pathway associated with leptin. The metabolic pathways or signaling pathways associated with leptin include, but are not limited to, the amyloidogenic pathways (which lead to generation of the Aβ peptide), the LRP pathway (which leads to endocytosis/clearance of the Aβ peptide), the insulin degrading pathway (which leads to degradation of the Aβ peptide), and any other pathway(s) affected by, or associated with, leptin. (See FIG. 2 for signaling pathways associated with leptin).

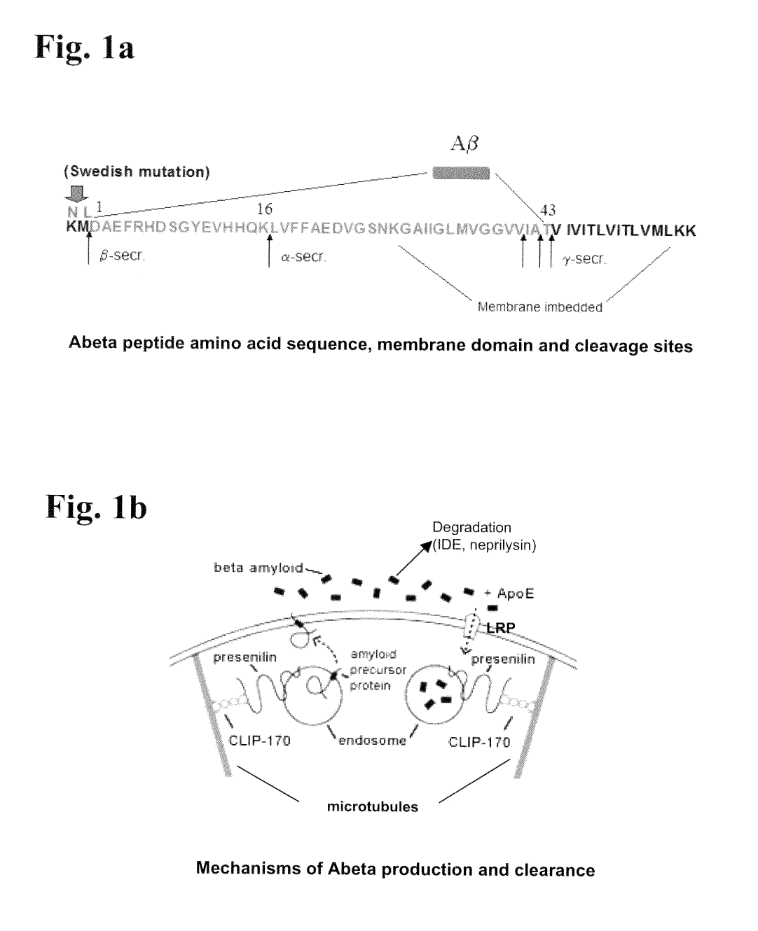

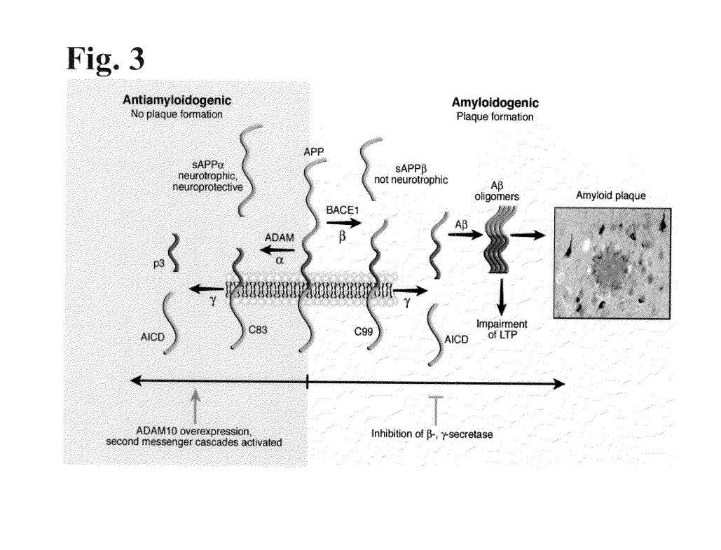

The term “amyloidogenic pathway” as used herein refers to the cellular mechanisms by which APP is proteolytically processed to generate amyloid-β, as shown in FIGS. 1 and 3. APP is proteolytically processed either through the amyloidogenic pathway or the antiamyloidogenic pathway. The term “fragment” or “peptide fragment” as used herein refers to a small part derived, cut off, or broken from a larger peptide, polypeptide or protein, which retains the desired biological activity of the larger peptide, polypeptide or protein. In the amyloidogenic pathway, consecutive cleavage of APP by β- and γ-secretase generates Aβ. In the amyloidogenic pathway, cleavage of APP by the protease β-secretase (BACE1) occurs at the N-terminus of the Aβ domain to yield the secreted sAPPβ (SEQ ID NO:7) as well as a C-terminal fragment of APP of 99 amino acids (C99) (SEQ ID NO:8). C99 is further cleaved within its transmembrane domain by y-secretase, leading to the secretion of the Aβ peptide and the generation of the APP intracellular domain (AICD). The Aβ peptide so generated is prone to aggregation. Aβ peptide oligomers are neurotoxic and lead to an impairment of long-term potentiation (LTP). Finally, large amounts of Aβ peptide are deposited in amyloid plaques, which are the characteristic pathological hallmarks of AD.

In the anti-amyloidogenic pathway, cleavage of APP by α-secretase within the AB peptide domain yields the neurotrophic and neuroprotective sAPPα. The α-secretase is a member of the ADAM (A Disintegrin And Metalloproteinase) family of metalloproteases. α-Cleavage of APP can be induced upon overexpression of ADAM10 or by the activation of second messenger cascades.

As used herein, the term “lipoprotein receptor related protein (LRP) pathway” refers to the pathway in neurons whereby the LDL receptor-related protein (LRP) modulates Aβ deposition. In neurons, the major apoE receptor is the LDL receptor-related protein (LRP), a large endocytic receptor that regulates proteinase and lipoprotein levels by mediating their catabolism. LRP modulates Aβ deposition by increasing its clearance and by serving as a receptor for APP, apoE, and alpha 2-macroglobulin (α2M), all of which have been genetically linked to AD. (Paula G. Ulery and Dudley K. Strickland, J Clin Invest. 106(9): 1077-1079 (2000)). It is believed that LRP is involved in the pathobiology of AD.

As used herein the term “insulin degrading pathway” refers to the pathway by which insulin-degrading enzyme (IDE), a 110-kDa metalloendopeptidase, degrades Aβ peptides.

The described invention also provides a method for diagnosing a cognitive disorder, disease, condition or precondition comprising measuring circulating leptin levels.

The described invention also provides methods of improving cognitive function in a subject in need thereof, the method comprising the step of administering to the subject (i) a composition comprising (i) leptin, a leptin mimic, a leptin derivative, a leptin agonist, an AMP-dependent protein kinase (AMPK) activator, or a leptin blocker, a mimic of a leptin blocker, a leptin antagonist, or an AMPK inhibitor and (ii) a pharmaceutically acceptable carrier to the subject. As used herein, the term “cognitive function” is as defined in above to refer to the intellectual processes resulting in an understanding, perception, or awareness of one's ideas as well as the ability to perform mental tasks, such as thinking, learning, judging, remembering, computing, controlling motor functions, and the like. The expression “resilience of cognitive function” refers to the ability of functional elements of cognitive function to resist deterioration over time. As used herein, the term “cognitive function enhancing amount” refers to that amount of the compositions of the described invention that will noticeably impact the ability to perform mental tasks, as measured by tests for memory, computation, attention, or other mental or cognitive attribute, or as suggested by an individual's perception of his or her abilities in these realms.

According to the described invention, the compositions of the invention may be administered orally, buccally, parenterally, intranasally, rectally, or topically.

The compositions of the described invention may be in a form suitable for oral use, for example, as tablets, troches, lozenges, aqueous or oily suspensions, dispersible powders or granules, emulsions, hard or soft capsules or syrups or elixirs. As used herein, the terms “oral” or “orally” refer to the introduction into the body by mouth whereby absorption occurs in one or more of the following areas of the body: the mouth, stomach, small intestine, lungs (also specifically referred to as inhalation), and the small blood vessels under the tongue (also specifically referred to as sublingually). Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient(s) in admixture with non-toxic pharmaceutically-acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They also may be coated for controlled release.

Compositions of the described invention also may be formulated for oral use as hard gelatin capsules, where the active ingredient(s) is(are) mixed with an inert solid diluent, for example, calcium carbonate, calcium phosphate or kaolin, or soft gelatin capsules wherein the active ingredient(s) is (are) mixed with water or an oil medium, for example, peanut oil, liquid paraffin, or olive oil.

The compositions of the described invention may be formulated as aqueous suspensions wherein the active ingredient(s) is (are) in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth, and gum acacia; dispersing or wetting agents may be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions also may contain one or more coloring agents, one or more flavoring agents, and one or more sweetening agents, such as sucrose or saccharin.

Compositions of the described invention may be formulated as oily suspensions by suspending the active ingredient in a vegetable oil, for example arachis oil, olive oil, sesame oil or coconut oil, or in a mineral oil, such as liquid paraffin. The oily suspensions may contain a thickening agent, for example, beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Compositions of the described invention may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients, or example, sweetening, flavoring and coloring agents also may be present.

The compositions of the invention also may be in the form of an emulsion. An emulsion is a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will not occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil. Thus, the compositions of the invention may be in the form of an oil-in-water emulsion. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also may contain sweetening and flavoring agents.

The compositions of the invention also may be formulated as syrups and elixirs. Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded (meaning torn or cut) tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

For buccal administration, the compositions of the described invention may take the form of tablets or lozenges formulated in a conventional manner.

The compositions of the described invention may be in the form of a sterile injectable aqueous or oleaginous suspension. The term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord), intrasternal injection, or infusion techniques. A parenterally administered composition of the described invention is delivered using a needle, e.g., a surgical needle. The term “surgical needle” as used herein, refers to any needle adapted for delivery of fluid (i.e., capable of flow) compositions of the described invention into a selected anatomical structure. Injectable preparations, such as sterile injectable aqueous or oleaginous suspensions, may be formulated according to the known art using suitable dispersing or wetting agents and suspending agents.

The sterile injectable preparation also may be a sterile injectable solution or suspension in a nontoxic parenterally acceptable diluent or solvent, for example, as a solution in 1, 3-butanediol. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it doesn't rapidly settle out. In everyday life, the most common suspensions are those of solids in liquid water. Among the acceptable vehicles and solvents that may be employed are water, Ringer's solution, and isotonic sodium chloride solution. In addition, sterile, fixed oils are conventionally employed as a solvent or suspending medium. For parenteral application, particularly suitable vehicles consist of solutions, for example, oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers.

The compositions of the described invention may be in the form of a dispersible dry powder for delivery by inhalation or insufflation (either through the mouth or through the nose). Dry powder compositions may be prepared by processes known in the art, such as lyophilization and jet milling, as disclosed in International Patent Publication No. WO 91/16038 and as disclosed in U.S. Pat. No. 6,921,527, the disclosures of which are incorporated by reference. Spray drying, for example, is a process in which a homogeneous aqueous mixture of drug and the carrier is introduced via a nozzle (e.g., a two fluid nozzle), spinning disc or an equivalent device into a hot gas stream to atomize the solution to form fine droplets. The aqueous mixture may be a solution, suspension, slurry, or the like, but needs to be homogeneous to ensure uniform distribution of the components in the mixture and ultimately the powdered composition. The solvent, generally water, rapidly evaporates from the droplets producing a fine dry powder having particles from about 1 pm to 5 pm in diameter. The spray drying is done under conditions that result in a substantially amorphous powder of homogeneous constitution having a particle size that is respirable, a low moisture content and flow characteristics that allow for ready aerosolization. For example, the particle size of the resulting powder is such that more than about 98% of the mass is in particles having a diameter of about 10 pm or less with about 90% of the mass being in particles having a diameter less than 5 pm. Alternatively, about 95% of the mass will have particles with a diameter of less than 10 pm with about 80% of the mass of the particles having a diameter of less than 5 pm. Dry powder compositions also may be prepared by lyophilization and jet milling, as disclosed in International Patent Publication No. WO 91/16038, the disclosure of which are incorporated by reference.

The term “dispersibility” or “dispersible” means a dry powder having a moisture content of less than about 10% by weight (% w) water, according to some embodiments below about 5% w and in some embodiments less than about 3% w; a particle size of about 1.0-5.0 Am mass median diameter (MMD), according to some embodiments 1.04.0 pm MMD, and in some embodiments 1.0-3.0 pm MMD; a delivered dose of about >30%, according to some embodiments >40%, according to some embodiments >50%, and in some embodiments >60%; and an aerosol particle size distribution of about 1.0-5.0 μm mass median aerodynamic diameter (MMAD), according to some embodiments 1.5-4.5 fun MMAD, and in some embodiments 1.5-4.0 μm MMAD. Methods and compositions for improving dispersibility are disclosed in U.S. application Ser. No. 08/423,568, filed Apr. 14, 1995, the disclosure of which is hereby incorporated by reference.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” According to some embodiments, the average particle size is less than about 10 microns (μm) in diameter with a relatively uniform spheroidal shape distribution. According to some embodiments, the diameter is less than about 7.5 and, according to some embodiments, less than about 5.0 μm. According to some embodiments, the particle size distribution is between about 0.1 μm and about 5 μm in diameter; according to some embodiments the particle size distribution is about 0.3 μm to about 5 μm.

The term “dry” means that the composition has a moisture content such that the particles are readily dispersible in an inhalation device to form an aerosol. This moisture content is generally below about 10% by weight (% w) water, according to some embodiments below about 5% w, and according to some embodiments less than about 3% w.

The amount of the pharmaceutically acceptable carrier is that amount needed to provide the necessary stability, dispersibility, consistency and bulking characteristics to ensure a uniform pulmonary delivery of the composition to a subject in need thereof. Numerically the amount may be from about 0.05% w to about 99.95% w, depending on the activity of the drug being employed. According to some embodiments about 5% w to about 95% will be used. The carrier may be one or a combination of two or more pharmaceutical excipients, but generally will be substantially free of any “penetration enhancers.” Penetration enhancers are surface active compounds which promote penetration of a drug through a mucosal membrane or lining and are proposed for use in intranasal, intrarectal, and intravaginal drug formulations. Exemplary penetration enhancers include bile salts, e.g., taurocholate, glycocholate, and deoxycholate; fusidates, e.g., taurodehydrofusidate; and biocompatible detergents, e.g., Tweens, Laureth-9, and the like. The use of penetration enhancers in formulations for the lungs, however, is generally undesirable because the epithelial blood barrier in the lung can be adversely affected by such surface active compounds. The dry powder compositions of the described invention are readily absorbed in the lungs without the need to employ penetration enhancers.

The types of pharmaceutical excipients that are useful as carriers for pulmonary delivery include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable for pulmonary delivery include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-i3-cyclodextrin; and polysaccharides, such as raffinose, maltodextrins, dextrans, and the like; alditols, such as mannitol, xylitol, and the like. A preferred group of carbohydrates includes lactose, trehalose, raffinose, maltodextrins, and mannitol. Suitable polypeptides include aspartame. Amino acids include alanine and glycine, with glycine being preferred.

Additives, which are minor components of the composition for pulmonary delivery, may be included for conformational stability during spray drying and for improving dispersibility of the powder. These additives include hydrophobic amino acids such as tryptophan, tyrosine, leucine, phenylalanine, and the like.

For delivery by inhalation or insufflation, the composition of the described invention is placed within a suitable dosage receptacle in an amount sufficient to provide a subject with a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. Nos. 4,227,522; U.S. Pat. No. 4,192,309; and U.S. Pat. No. 4,105,027. Suitable containers also include those used in conjunction with Glaxo's Ventolin® Rotohaler brand powder inhaler or Fison's Spinhaler® brand powder inhaler. Another suitable unit-dose container which provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). All of these references are incorporated herein by reference.

The compositions of the invention may be used in the form of drops or sprays (e.g., a nasal spray, aerosol spray, or pump spray) or other vehicles for nasal administration (intranasal delivery). Aerosol spray preparations can be contained in a pressurized container with a suitable propellant such as a hydrocarbon propellant. Pump spray dispensers can dispense a metered dose or a dose having a specific particle or droplet size. Any dispensing device can be arranged to dispense only a single dose, or a multiplicity of doses. More generally, compositions of the invention, especially those formulated for intranasal administration, can also be provided as solutions, suspensions, or viscous compositions (e.g., gels, lotions, creams, or ointments).

The compositions of the described invention may be in the form of suppositories for rectal administration of the composition. “Rectal” or “rectally” as used herein refers to introduction into the body through the rectum where absorption occurs through the walls of the rectum. These compositions can be prepared by mixing the drug with a suitable nonirritating excipient such as cocoa butter and polyethylene glycols which are solid at ordinary temperatures but liquid at the rectal temperature and will therefore melt in the rectum and release the drug. When formulated as a suppository the compositions of the invention may be formulated with traditional binders and carriers, such as triglycerides.

The term “topical” refers to administration of an inventive composition at, or immediately beneath, the point of application. The phrase “topically applying” describes application onto one or more surfaces(s) including epithelial surfaces. Although topical administration, in contrast to transdermal administration, generally provides a local rather than a systemic effect, as used herein, unless otherwise stated or implied, the terms topical administration and transdermal administration are used interchangeably. For the purpose of this application, topical applications shall include mouthwashes and gargles.

Topical administration may also involve the use of transdermal administration such as transdermal patches or iontophoresis devices which are prepared according to techniques and procedures well known in the art. The terms “transdermal delivery system,” transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for postmenopausal indications, and nicotine for smoking cessation.

Patches suitable for use in the described invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, 249-297 (Tapash K. Ghosh et al. eds., 1997), hereby incorporated herein by reference. These patches are well known in the art and generally available commercially.

In some embodiments, the compositions of the described invention may be formulated with an excipient, vehicle or carrier selected from solvents, suspending agents, binding agents, fillers, lubricants, disintegrants, and wetting agents/surfactants/solubilizing agents. The terms “excipient”, “vehicle”, or “carrier” refer to substances that facilitate the use of, but do not deleteriously react with, the active compound(s) when mixed with it. The term “active” refers to the ingredient, component or constituent of the compositions of the described invention responsible for the intended therapeutic effect. Carriers must be of sufficiently high purity and of sufficiently low toxicity to render them suitable for administration to the subject being treated. The carrier can be inert, or it can possess pharmaceutical benefits.

The carrier can be liquid or solid and is selected with the planned manner of administration in mind to provide for the desired bulk, consistency, etc., when combined with an active and the other components of a given composition. Typical pharmaceutical carriers include, but are not limited to, binding agents (including, but not limited to pregelatinized maize starch, polyvinylpyrrolidone or hydroxypropyl methylcellulose); fillers (including but not limited to lactose and other sugars, microcrystalline cellulose, pectin, gelatin, calcium sulfate, ethyl cellulose, polyacrylates or calcium hydrogen phosphate.); lubricants (including, but not limited to magnesium stearate, talc, silica, sollidal silicon dioxide, stearic acid, metallic stearates, hydrogenated vegetable oils, corn starch, polyethylene glycols, sodium benzoate, sodium acetate); disintegrants (including but not limited to starch, sodium starch glycolate) and wetting agents (including but not limited to sodium lauryl sulfate). Additional suitable carriers for the compositions of the described invention include, but are not limited to, water, salt solutions, alcohol, vegetable oils, polyethylene glycols, gelatin, lactose, amylose, magnesium stearate, talc, silicic acid, viscous paraffin, perfume oil; fatty acid monoglycerides and diglycerides, petroethral fatty acid esters, hydroxymethylcellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds.

The term “pharmaceutically acceptable carrier” as used herein refers to any substantially non-toxic carrier conventionally useful for administration of pharmaceuticals in which the active component will remain stable and bioavailable. In some embodiments, the pharmaceutically acceptable carrier of the compositions of the described invention include a release agent such as a sustained release or delayed release carrier. In such embodiments, the carrier can be any material capable of sustained or delayed release of the leptin peptide active ingredient to provide a more efficient administration, resulting in less frequent and/or decreased dosage of the active ingredient, ease of handling, and extended or delayed effects. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines.

The therapeutically active leptin, leptin mimic, leptin agonist, or leptin derivative peptides, as well as leptin blockers and leptin antagonists of the described invention can be formulated per se or in salt form. The term “pharmaceutically acceptable salts” refers to nontoxic salts of the peptides of the described invention. The peptide salts which can be used for the invention are pharmaceutically acceptable salts of organic acids or pharmaceutically acceptable salts of inorganic acids. Examples of such pharmaceutically acceptable peptide salts include, but are not limited to, those formed with free amino groups such as those derived from hydrochloric, phosphoric, sulfuric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Additional compositions of the described invention can be prepared readily using technology is known in the art, such as that which is described in Remington's Pharmaceutical Sciences, 18th or 19th editions, published by the Mack Publishing Company of Easton, Pa., which is incorporated herein by reference.

In some embodiments, the compositions of the described invention can further include one or more compatible active ingredients aimed at providing the composition with another pharmaceutical effect in addition to that provided by a leptin, leptin mimic peptide or a derivative thereof “Compatible” as used herein means that the active ingredients of such a composition are capable of being combined with each other in such a manner so that there is no interaction that would substantially reduce the efficacy of each active ingredient or the composition under ordinary use conditions. In another aspect of the described invention, the composition also may be administered serially or in combination with other compositions for treating diseases, conditions or disorders resulting from accumulation of amyloid peptides. For example, without limitation, such other compositions may include monoclonal antibodies (such as monoclonal anti-β-Amyloids and monoclonal anti-β-secretases); and anti-inflammatory compounds (including, but not limited to nonsteroidal anti-inflammatory drugs (NSAIDs), such as ibuprofen, indomethacin, and flurbiprofen). Anti-inflammatory compounds have been shown to direct Aβ-lowering properties in cell cultures as well as in transgenic models of AD-like amyloidosis.

A composition of the described invention, alone or in combination with other active ingredients, may be administered to a subject in a single dose or multiple doses over a period of time. As used herein, the terms “therapeutically effective amounts,” and “pharmaceutically effective amounts” are used interchangeably to refer to the amount of the composition of the invention that results in a therapeutic or beneficial effect, including a subject's perception of health or general well-being, following its administration to a subject. Additionally, the terms “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition resulting from accumulation of an amyloid peptide in an amount sufficient to eliminate or reduce the risk, lessen the severity, or delay the onset of the disease, disorder or condition, including biochemical, histologic and/or behavioral symptoms of the disease, disorder or condition, its complications and intermediate pathological phenotypes presenting during development of the disease, disorder or condition.

The concentration of the active substance is selected so as to exert its therapeutic effect, but low enough to avoid significant side effects within the scope and sound judgment of the skilled artisan. The effective amount of the composition may vary with the age and physical condition of the biological subject being treated, the severity of the condition, the duration of the treatment, the nature of concurrent therapy, the specific compound, composition or other active ingredient employed, the particular carrier utilized, and like factors. Those of skill in the art can readily evaluate such factors and, based on this information, determine the particular effective concentration of a composition of the described invention to be used for an intended purpose. Additionally, in therapeutic applications of the described invention, compositions or medicants are administered to a patient suspected of, having, or already suffering from, such a disease, disorder or condition in an amount sufficient to cure, or at least partially arrest, the symptoms of the disease, disorder or condition, including its complications and intermediate pathological phenotypes in development of the disease, disorder or condition. In some methods, administration of the composition of the described invention reduces or eliminates cognitive impairment in patients that have not yet developed characteristic pathology of the disease, disorder or condition.

An amount adequate to accomplish therapeutic or prophylactic treatment is defined herein as a therapeutically-effective dose. In both prophylactic and therapeutic regimes, an amount of the compositions of the described invention is usually administered in several dosages until a sufficient beneficial response has been achieved. Typically, the response is monitored and repeated dosages are given if the response starts to wane. A skilled artisan can determine a pharmaceutically effective amount of the inventive compositions by determining the dose in a dosage unit (meaning unit of use) that elicits a given intensity of effect, hereinafter referred to as the “unit dose.” The term “dose-intensity relationship” refers to the manner in which the intensity of effect in an individual recipient relates to dose. The intensity of effect generally designated is 50% of maximum intensity. The corresponding dose is called the 50% effective dose or individual ED50. The use of the term “individual” distinguishes the ED50 based on the intensity of effect as used herein from the median effective dose, also abbreviated ED50, determined from frequency of response data in a population. “Efficacy” as used herein refers to the property of the compositions of the described invention to achieve the desired response, and “maximum efficacy” refers to the maximum achievable effect. The amount of compounds in the compositions of the described invention which will be effective in the treatment of a particular disorder or condition will depend on the nature of the disorder or condition, and can be determined by standard clinical techniques. (See, for example, Goodman and Gilman's THE PHARMACOLOGICAL BASIS OF THERAPEUTICS, Joel G. Harman, Lee E. Limbird, Eds.; McGraw Hill, New York, 2001; THE PHYSICIAN'S DESK REFERENCE, Medical Economics Company, Inc., Oradell, N.J., 1995; and DRUG FACTS AND COMPARISONS, FACTS AND COMPARISONS, INC., St. Louis, Mo., 1993). The precise dose to be employed in the formulation will also depend on the route of administration, and the seriousness of the disease or disorder, and should be decided according to the judgment of the practitioner and each patient's circumstances. Various administration patterns will be apparent to those skilled in the art.

The term “phosphorylated tau accumulation modulating amount” as used herein refers to a therapeutically effective amount of a leptin composition that modulates the phosphorylation of tau protein. A phosphorylated tau accumulation modulating amount includes prophylactic or preventative amounts of the compositions of the described invention.

The dosage ranges for the administration of the compositions of the described invention are those large enough to produce the desired therapeutic effect. According to some embodiments, the therapeutically effective amount of the compositions of the described invention is administered one or more times per day on a regular basis. A typical dose administered to a subject is between about 0.01 mg of the composition per kg (of body weight) per day and about 0.5 mg of the composition per kg (of body weight) per day. For example, without limitation, the minimum dose of the composition is contemplated as about 0.01 mg/kg/day, about 0.025 mg/kg/day, about 0.05 mg/kg/day, about 0.075 mg/kg/day, about 0.08 mg/kg/day, about 0.1 mg/kg/day, about 0.125 mg/kg/day, about 0.15 mg/kg/day, about 0.175 mg/kg/day, about 0.2 mg/kg/day, about 0.225 mg/kg/day, about 0.25 mg/kg/day, about 0.275 mg/kg/day, about 0.3 mg/kg/day, about 0.325 mg/kg/day, about 0.35 mg/kg/day, about 0.375 mg/kg/day, about 0.4 mg/kg/day, about 0.45 mg/kg/day, about 0.475 mg/kg/day, or about 0.5 mg/kg/day and the maximum dose is contemplated as about 0.5 mg/kg/day, about 0.475 mg/kg/day, about 0.45 mg/kg/day, about 0.4 mg/kg/day, about 0.375 mg/kg/day, about 0.35 mg/kg/day, about 0.325 mg/kg/day, about 0.3 mg/kg/day, about 0.275 mg/kg/day, about 0.25 mg/kg/day, bout 0.225 mg/kg/day, about 0.2 mg/kg/day, about 0.175 mg/kg/day, about 0.15 mg/kg/day, about 0.125 mg/kg/day, about 0.1 mg/kg/day, about 0.08 mg/kg/day, about 0.075 mg/kg/day, about 0.05 mg/kg/day, about 0.025 mg/kg/day, or about 0.01 mg/kg/day. In some embodiments of the invention in humans, the dose may be about 0.01 mg to about 0.3 mg of the composition per kg (of body weight) per day, and in other embodiments in humans, between 0.01 and 0.08 mg of the composition per kg (of body weight) per day.

Those skilled in the art will recognize that initial indications of the appropriate therapeutic dosage of the compositions of the invention can be determined in in vitro and in vivo animal model systems, and in human clinical trials. One of skill in the art would know to use animal studies and human experience to identify a dosage that can safely be administered without generating toxicity or other side effects. For acute treatment, it is preferred that the therapeutic dosage be close to the maximum tolerated dose. For chronic preventive use, lower dosages may be desirable because of concerns about long term effects.

The effectiveness of the compositions and methods of the described invention can be assayed by a variety of protocols. The effects of increasing cognitive function in a human subject can be determined by methods routine to those skilled in the art including, but not limited to, both paper and pencil, and computer tests. One of skill in the art can also directly measure amyloid peptide accumulation levels, neurofibrillary tangle formation and neurodegeneration in animal models. Furthermore, amyloid peptide may be measured in a sample of a subject's cerebrospinal fluid (CSF) obtained by spinal tap. One measure of accumulation of an amyloid peptide is an increase in levels circulating in the blood of a subject. Such levels may be measured by Sandwich Enzyme-linked-Immunoabsorbent-Assays (ELISAs), using a pair of antibodies, one for capture and the other for detection. These methods are well known by those of ordinary skill in the art.

The term “variant” as used herein refers to a peptide sequence that varies at one or more amino acid positions with respect to the reference peptide. A variant can be a naturally-occurring variant or can be the result of spontaneous, induced, or genetically engineered mutation(s) to the nucleic acid molecule encoding the variant peptide. A variant peptide can also be a chemically synthesized variant. A skilled artisan likewise can produce polypeptide variants having single or multiple amino acid substitutions, deletions, additions or replacements. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, such as, for example, an epitope for an antibody. The techniques for obtaining such variants, including genetic (suppressions, deletions, mutations, etc.), chemical, and enzymatic techniques are known to the skilled artisan.

The leptin of the described invention may be altered in various ways including amino acid substitutions, deletions, truncations (e.g, fragments), and insertions. Methods for such manipulations are generally known in the art. For example, amino acid sequence variants can be prepared by mutations in the DNA. Methods for mutagenesis and nucleotide sequence alterations are well known in the art. For example, point mutations may be introduced into the leptin coding sequence using Invitrogen's GeneTaylor Mutagenesis System, Stratagene's QuickChange System or NEB's Phusion System according to manufacturer's guidelines. The described invention is intended to encompass conservative substitutions, such as exchanging one amino acid with another amino acid having similar properties without altering peptide structure and/or function.

The described invention provides a recombinant leptin. Methods for producing recombinant peptides are well-known in the art. For example, the nucleic acid encoding leptin of the described invention may be inserted into a replicable vector for cloning (amplification of the DNA) or for expression. Various vectors are publicly available. The vector may, for example, be in the form of a plasmid, cosmid, viral particle, or phage. For example, plasmid vectors include, but are not limited to, pET-26+ and pCMV6-AC. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

A number of promoters can be used in the practice of the invention. For example, a promoter can be employed which will direct expression of a polynucleotide of the described invention in E. coli. Other equivalent transcription promoters from various sources are known to those of skill in the art. By way of non-limiting example, expression and cloning vectors usually contain a promoter operably linked to the leptin encoding nucleic acid sequence to direct mRNA synthesis. Promoters recognized by a variety of potential host cells are well known. Promoters suitable for use with prokaryotic hosts include the beta-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21-25 (1983)). Promoters for use in bacterial systems also will contain a Shine-Dalgarno (S.D.) sequence operably linked to the DNA encoding leptin.

Both expression and cloning vectors contain a nucleic acid sequence that enables the vector to replicate in one or more selected host cells. Such sequences are well known for a variety of bacteria, yeast, and viruses.

Expression and cloning vectors will typically contain a selection gene, also termed a selectable marker. Typical selection genes encode proteins that (a) confer resistance to antibiotics or other toxins (e.g., ampicillin, neomycin, methotrexate, or tetracycline), (b) complement auxotrophic deficiencies, or (c) supply critical nutrients not available from complex media, (e.g., the gene encoding D-alanine racemase for Bacilli). Examples of suitable selectable markers for mammalian cells include, but are not limited to, those that enable the identification of cells competent to take up the leptin-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

The leptin of the described invention may be produced recombinantly not only directly, but also as a fusion peptide with a heterologous peptide. The heterologous peptide may include, but is not limited to, an IgG1-Fc, transferrin, a signal sequence or other peptide having a specific cleavage site at the N-terminus of the mature protein or polypeptide. By way of non-limiting example, leptin of the described invention may be cloned into the multiple cloning site of the pFUSE-hIgG1-Fc vector. For leptin fusion to the human transferrin protein, for example, the hIgG1-Fc nucleotide sequence may be excised by restriction endonuclease from the pFUSE-hIgG1-Fc vector and human transferrin nucleotide sequence inserted.

Host cells are transfected or transformed with expression or cloning vectors described herein for leptin production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991).

Methods of eukaryotic cell transfection and prokaryotic cell transformation are known to the ordinarily skilled artisan, for example, CaCl₂, Ca₂PO₄, liposome-mediated and electroporation. Depending on the host cell used, transformation is performed using standard techniques appropriate to such cells. The calcium treatment employing calcium chloride, as described in Sambrook et al., or electroporation is generally used for prokaryotes. For mammalian cells, the calcium phosphate precipitation method of Graham and van der Eb, Virology, 52:456-457 (1978) can be employed. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, See Keown et al., Methods in Enzymology, 185:527-537 (1990) and Mansour et al., Nature, 336:348-352 (1988).

Suitable host cells for cloning or expressing DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and KS 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. These examples are illustrative rather than limiting.

Addition of isopropyl-beta-D-thiogalactopyranoside (IPTG) to bacterial cultures is a long-standing way to induce expression of plasmid-based genes for the production of recombinant peptides under the control of the lac promoter. IPTG binds to the lac repressor in Escherichia coli, thereby preventing binding of the repressor protein to DNA and blocking gene transcription.

After expression, recombinant leptin may be recovered from culture medium or from host cell lysates. If membrane-bound, it can be released from the membrane using a suitable detergent solution (e.g. Triton-X 100) or by enzymatic cleavage. Cells employed in expression of leptin can be disrupted by various physical or chemical means, such as freeze-thaw cycling, sonication, mechanical disruption, or cell lysing agents.

It may be desired to isolate or purify leptin from recombinant cell proteins or polypeptides. The following procedures are exemplary of suitable isolation and purification procedures: by fractionation on an ion-exchange column; reverse phase HPLC; chromatography on silica or on a cation-exchange resin such as DEAE; chromatofocusing; SDS-PAGE; ammonium sulfate precipitation; metal chelating columns; or a specific antibody column to bind epitope-tagged forms of the protein of interest (e.g., nickel columns to bind His-tagged proteins, anti-HA columns to bind HA-tagged proteins, or anti-FLAG columns to bind FLAG-tagged proteins). Various methods of protein purification may be employed, and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular peptide produced.

According to one embodiment, the described invention provides a route of administrating the composition. The composition may be constituted into any form suitable for the mode of administration selected. Exemplary routes of administration include, but are not limited to, parenteral (including subcutaneous), oral, inhalation, insufflation, topical, buccal and rectal. Compositions suitable for parenteral administration include sterile solutions, emulsions and suspensions. Oral administration include solid forms, such as pills, capsules, granules, tablets, and powders, and liquid forms, such as solutions, syrups, elixirs, and suspensions. Compositions suitable for inhalation and insufflation may take the form of an aerosolized solution. Compositions suitable for topical administration include creams, ointments and dermal patches. Compositions suitable for buccal administration may take the form of tablets or lozenges. Compositions suitable for rectal administration may take the form of suppositories. Formulations for administration may be provided using any formulation known in the art and appropriate for the route of administration. Such formulations may be as provided using the guidance of such resources as REMINGTON'S PHARMACEUTICAL SCIENCES, 18th ed., Mack Publishing Co., Easton, Pa. 1990.

According to another embodiment, the described invention provides for a composition comprising a leptin and a pharmaceutically acceptable carrier. Pharmaceutically acceptable carriers encompass any of the standard pharmaceutical carriers. For example, pharmaceutically acceptable excipients may include a solution that is isotonic with blood such as saline, Ringer's solution, or dextrose solution. Alternatively, non-aqueous vehicles such as fixed oils and ethyl oleate may be used, as well as liposomes. Further, excipients may be included that improve the efficacy, receptor affinity, or half-life of the active ingredient. For example, but not by way of limitation, the EPO-derived peptides of the methods of the described invention may be pegylated (i.e., coupled with polyethylene glycol) by means well-known in the art to prolong the half-life of the active ingredient in the circulation. (See, e.g., Kozlowski et al. J. Control Release 72: 217-224, 2001). Such modification may enhance biological activity to be useful as therapeutic agents.

According to one embodiment, the first composition comprises a leptin derivative, exemplified by a truncated leptin protein, or a leptin with unnatural amino acid substitutions, or other modifications that may alter leptin potency.

According to one embodiment, the leptin may be delivered through gene-therapy, including the use of viral vectors, like adeno-associated virus, herpes simplex virus, or retroviral vectors like lentiviral vectors which can encode the leptin therapeutic wherein the viral vector can be either by systemic or stereotactically administered into the CNS, wherein the therapeutic amount of the leptin or the leptin mimic is effective to modulate accumulation of the amyloid peptide in brain, the hyperphosphorylation of tau or loss of neuronal function as it can occur in neurodegenerative disorders.

According to one embodiment, the method may further comprise monitoring circulating levels of the amyloid peptide or hyperphosphyrlated tau by imaging or direct measurement of these proteins in the cerebral spinal fluid (CSF). According to another embodiment, the circulating levels of amyloid peptide may be detected in a sample of CSF or blood.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the described invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “an”, and “the” include plural references unless the context clearly dictates otherwise. All technical and scientific terms used herein have the same meaning.

The publications discussed herein are provided solely for their disclosure prior to the filing date of the present application and each is incorporated by reference in its entirety. Nothing herein is to be construed as an admission that the described invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Methods

Reagents.

Minimum essential medium (MEM) was purchased from ATCC (Manassas, Va.). Trypsin-EDTA and penicillin solution were purchased from MP Biomedicals (Solon, Ohio). Fetal bovine serum (FBS), all-trans retinoic acid (ATRA), G418, water-soluble cholesterol, water-soluble oleic acid and recombinant human leptin were purchased from Sigma-Aldrich (St. Louis, Mo.). C₂ ceramide was purchased from EMD Biosciences (San Diego, Calif.). Leptin fragments (Lep 22-56; Tyr-Lep 26-39; Lep 93-105) were purchased from CPC Scientific (Sunnyvale, Calif.).

Cell Cultures, Treatments, Antibodies and Immunoassays.

SY5Y cells (human neuroblastoma) were maintained in culture as described (Johnsingh et al., FEBS Lett. 465:53-8 (2000)). The human neuroblastoma cell line, SH-SY5Y, was purchased from ATCC. Cell culture was performed according to manufacturer's specific guidelines. Cells were propagated in MEM containing 10% FBS. Neuronal differentiation was performed as described previously (Greco, S. J. et al., Biochem Biophys Res Commun 376 (2008) 536-541). To generate SY5Y stably over-expressing amyloid precursor protein (APP), cells were transfected with a mammalian expression vector encoding the 751 amino acid isoform of human APP (APP₇₅₁—Accession # NM_{—}201413) (Origene Technologies; Rockville, Md.) using the FuGENE HD transfection reagent, according to manufacturer's specific instructions (Promega; Madison, Wis.). Briefly, cells were transiently transfected with APP₇₅₁ or vehicle for 48 h and then switched into selection medium containing a concentration range of the antibiotic G418 (100-600 μg/mL) to determine the optimal dose for stable selection. Selection media was changed every 3 days to remove non-viable cells. After 3 weeks, 200 μg/mL G418 yielded distinct colonies while all vehicle-transfected cells were non-viable. Cells were maintained in 10% FBS media containing 200 μg/mL G418 for expansion. Primary neural cultures were obtained from E 16 rat embryonic cortex, as described (Shimoda et al., 1992. Brain Res. 586:319-31 (1992)). These were either grown as mixed cultures (Johnsingh et al., FEBS Lett. 465:53-8 (2000)) or grown under conditions that favor the isolation and proliferation of astrocytes (Takeshima et al., J Neurosci. 14:4769-79 (1994)).

Neuro2a (mouse neuroblastoma) stably transfected with hyg-sa134, a pcDNA3.1/Hygro plasmid (Invitrogen, CA) modified to express a fusion protein of secreted alkaline phosphatase (SEAP) and a fragment of APP consisting of the C-terminal 134 amino acids (“CAPP134”) (SEQ ID NO:10) were maintained in culture as described (Johnsingh et al., J Neurosci. 14:4769-79 (2000)) in the presence of 400 μg/ml of hygromycin. SEQ ID NO: 11 is the DNA sequence of the entire hygsa134 vector, which was derived from the pcDNA3.1/Hygro vector by genetic manipulation to insert the DNA sequences for SEAP and CAPP 134—The SEAPCAPP cDNA insert from hyg-sal 34 was also subcloned into an adenoviral vector using the Adeno Vator system (Qbiogene, CA). The DNA sequence for SEAP (corresponding to nucleotides 981-2441 of hyg-sa134) (SEQ ID NO: 9) is located 5′ to the DNA sequence (SEQ ID NO: 12) coding for CAPP134 (SEQ ID NO: 10).

SY5Y and hyg-sa134-Neuro2a cells were treated at 80% confluency (see below). Primary neural cultures from mouse embryos were allowed to grow for 6-12 days following plating and prior to viral infection and treatments.

About 5 μg/ml or about 10 μg/ml water-soluble cholesterol was added to cultures for 2 or 5 hours. Water soluble cholesterol (Sigma-Aldrich, MO) is a solution made of cholesterol balanced with cyclodextrin CDX (40 mg cholesterol per gr CDX). For comparison, cultures were treated with the equivalent amount of the resin alone, which leads to depletion of cholesterol in the cultures (Simons et al., Proc Natl Acad Sci USA 95:6460-4 (1998)).

About 100 ng/ml or about 400 ng/ml leptin (Harbor-UCLA, CA), was added in cell culture medium for 2 or 5 h. Cells were approximately 80% confluent at the time of treatment. Peptide YY (3-36) (Phoenix Pharmaceuticals, Inc., CA), and CNTF (Sigma-Aldrich, MO) were added at about 2511M or 150 1.IM for the same incubation periods. TOFA, etomoxir (Research Biochemicals International, MA) and cerulenin (Sigma-Aldrich, MO) were used as described below.

Cell lysates were used for the detection of full-length APP_(SEQ ID NO: 1-SEQ ID NO: 3) and its C-terminal fragments generated by 13- and a-secretase (10 kDa (SEQ ID NO: 8) and 8 kDa (SEQ ID NO: 16) respectively) as described (Johnsingh et al., FEBS Lett. 465:53-8 (2000)). This was performed either by ³⁵S-[Met]/³⁵S-[Cys] metabolic labeling/immunoprecipitations or Western blots using a rabbit polyclonal antibody directed against the last 20 C-terminal amino acids of APP (Institute for Basic Research, NY) (Figueiredo-Pereira et al., J Neurochem. 72:1417-22 (1999); Johnsingh et al., FEBS Lett. 465:53-8. (2002)).

For the determination of Aβ peptide several methodologies also were used. SY5Y cells in culture were metabolically labeled with ³⁵S-[Met] as described (Figueiredo-Pereira et al. J Neurochem. 72:1417-22 (1999)), followed by immunoprecipitation, resolution of the immunoprecipitates on SDS-PAGE, autoradiography, and densitometric analysis of the autoradiogram. Neuro2a cells were stably transfected with hyg-sa134 (K. Sambamurti, S. Carolina Medical Center, SC) and Aβ40 (SEQ ID NO: 4) plus Aβ42 (SEQ ID NO: 5) plus Aβ43 (SEQ ID NO: 6) (Total A(3) in the medium then was quantified by sandwich ELISAs developed with 4G8 and 6E10 monoclonal antibodies (Signet, MA) as described (Figueiredo-Pereira et al., J Neurochem. 72:1417-22 (1999)). Commercially available ELISA kits (KMI Diagnostics, MN) were used for the separate determination of Aβ40 (SEQ ID NO:4) and Aβ42/43 (SEQ ID NO:5/SEQ ID NO:6) in formic acid extracts of mice brains. Flotillin was detected using monoclonal anti-flotillin-1 antibodies (BD Biosciences, CA). Actin was detected using monoclonal anti-actin antibodies (Research Diagnostics, Inc, NJ).

Leptin was detected using a rabbit polyclonal antibody raised against mouse leptin, cross-reacting with human leptin (obtained from Dr. A. F. Parlow, Harbor-UCLA, CA). Immunofluorescent confocal microscopy was performed on 2% paraformaldehyde-fixed primary neural cells. Filipin staining was performed as described (Feng et al., Nat Cell Biol. 5:781-92 (2003)).

Preparation of ApoE and Binding with ¹²⁵I-Aβ.

ApoE was isolated from the conditioned media of human embryonic kidney (HEK-293) cells stably-transfected with human apoE (having the E3 allele or the E4 allele) cDNA (Tezapsidis et al., FASEB J. 17:1322-1324 (2003)). These preparations, while usually poor in lipid, are fully functional for uptake experiments. ApoE then was pre-incubated with ¹²⁵I-A13 overnight at 37° C. (Aβ/ApoE: 1/50 w/w) as described (Tezapsidis et al., FASEB J. 17:1322-1324 (2003)).

Aβ-Uptake by SY5Y Cells.

Human ¹²⁵I-AP (iodinated at Tyr-10, Amersham Biosciences, IM 294) uptake was measured following addition of 0.1 nM ¹²⁵-I-Aβ40 (SEQ ID NO: 4) to confluent SY5Y cells (60,000 cpm/ml) in the presence or absence of 100 ng/ml or 400 ng/ml leptin also included in a 24 h pre-incubation period. ¹²⁵I-Aβ was either added alone or was previously incubated with apoE3. In controls, Receptor Associated Protein (“RAP”, 1 μM) was added together with AP or the AP/apoE complex. RAP is an antagonist of a number of lipoprotein receptors (LaDu et al., Neurochem Int. 39:427-34 (2001)). After 24 h, the media were collected and subjected to scintillation counting for eradiation (Kang et al., 2000. J Clin Invest. 106:1159-66 (2000)). The amount of radioactivity was measured in both the trichloroacetic acid (10%) TCA pellets (representing intact Aβ) and the corresponding supernatants (representing degraded Aβ). 96.5±8.2% (mean±s.e.m., n=4 experiments, triplicate determinations) of the radioactivity found in the medium could be recovered in the TCA pellet and represented intact or oligomeric Ap (not shown), when Aβ was pre-incubated with apoE. However, only 31.2±5.8% (n=4) of the radioactivity was recovered in the TCA pellet in the absence of apoE, suggesting that AP was degraded under those conditions, consistent with reports by others. This has been suggested to be due to the activity of Insulin-Degrading Enzyme (Farris et al., Proc Natl Acad Sci USA. 100:4162-4167 (2003)). Indeed, inclusion during the uptake of 1,10 phenanthroline, a general metalloprotease inhibitor that effectively inhibits degradation of secreted Aβ in vitro, abolished AP degradation.

The amount of TCA-precipitable radioactivity in the soluble fraction of cell lysates was compared to that in the total lysates, the ratio of which was typically about 0.8 to about 0.9 (not shown), to further verify that radioactivity was reduced in the media as a reflection of AP uptake by the cells, rather than due to non-specific binding to the extracellular surface of membranes or oligomerization/aggregation of Aβ.

Measurement of Protein.

Proteins were extracted from cells by treatment with the nonionic surfactant Igepal (SIGMA, 0.1%) and brief sonication. Protein content was determined by the Bradford method (Bradford, Anal Biochem. 72:248-54 (1976)).

SREBP cDNAs.

Human SREBP-1 (SEQ ID NO: 17) and SREBP-2 (SEQ ID NO: 19) cDNAs were obtained by polymerase chain reaction (“PCR”) using a human brain cDNA expression library as a template. Briefly, 5′-gagaggatccaacagggcaggacacgaa-3′ (linker italicized, BamHI site underlined) (SEQ ID NO: 20) was used as forward primer and 5′-gagagaattcggctgctgccaagggaca-3′ (linker italicized, EcoRI site underlined) (SEQ ID NO: 21) was used as a reverse primer, generating a 1461 nt fragment of human SREBP-1 (GenBank Accession No. U00968, GenInfo Identifier (GI):409404) predicted to encode for SREBP-1 (1445 amino acids) (SEQ ID NO: 24). The resulting 1.5-kb fragment was cloned into the BamHI and EcoRI sites of the pcDNA3.1 vector. Similarly, 5′-gagaggatccaaggttgtcgggtgtcatg-3′ (linker italicized, BamHI site underlined) (SEQ ID NO: 22) was used as a forward primer and 5′-gagagaattcggctggctcatctttgacctt-3′ (linker italicized, EcoRI site underlined) (SEQ ID NO: 23) as a reverse primer, generating a 1492 nt fragment of human SREBP-2 (GenBank Accession No. UO2031, GI:451329), predicted to encode for SREBP-2 (1-467 amino acids) (SEQ ID NO: 25). The resulting 1.5-kb fragment was cloned into the BamHI and EcoRI sites of the pcDNA3.1 vector.

Leptin studies in mice. One year-old transgenic animals with the following genotypes were used: a) APP_{swe}/PS1_{M146V} (double transgenic) (Holcomb et al., Nat Med. 4:97100 (1998)); b) PS1_{M146V} (Duff et al., Nature. 383:710-3 (1996)) and c) wild-type C57B1/6×SJL. SEQ ID NO: 13 is the amino acid sequence for APPswe. A double mutation at codons 670 and 671 (APP isoform a) co-segregates with the disease in two large (probably related) early-onset Alzheimer's disease families from Sweden. Two base pair transversions (G to T, A to C) from the normal sequence predict L to N and M to L amino acid substitutions at codons 670 and 671 of the APP transcript. SEQ ID NO:14 is the amino acid sequence of PS 1 in humans. SEQ ID NO: 15 is the amino acid sequence of PS1_{M146V}. A single mutation at codon 146 co-segregates with the disease in members of early-onset Alzheimer's disease families. A base pair change from the normal sequence predicts M to V amino acid substitution at codon 146.

Blood was withdrawn (approximately 1 ml) from deeply anaesthetized animals by cardiocentesis and mixed with 25 μl of 164 μM EDTA anticoagulant. Plasma was prepared immediately and frozen at −70 C.°. Plasma leptin concentrations were determined by a radioimmunoassay (RIA) (Chung et al., Am. J. Physiol. 274:R985-R990 (1998)), using a kit from LINCO Research, Inc. (Missouri).

APP_{swe} (SEQ ID NO: 13) expressing mice (Tg2576) or wild-type littermates were maintained in pathogen-free environment at 25° C. on a 12-12 h light-dark cycle. Mice were euthanized between the ages of 31 and 40 weeks. They were provided ad libidum access for up to 9 weeks (i.e., 1 week prior to leptin treatments and 8 weeks during such treatments) to a high fat diet (D 12451) containing about 45% of the total calories from fat (Research Diets, NJ) or to a low fat diet (D 12450B) containing about 10% of the total calories from fat. Equal number of male and female Tg2576 mice under each diet were administered leptin or a placebo (PBS) from the age of 32 wks to up to 40 wks of age. For this, mice were anaesthetized with intraperitoneal injection of ketamine (55 mg/ml) and xylazine (7-10 mg/ml) and surgically fitted with an Alzet miniosmotic pump (model 2004, Durect Co, CA) placed subcutaneously. Local subcutaneous injection of 0.5 ml of 0.5% lidocaine insured postoperative relief. Half of the mice received daily about 20 pg leptin in PBS (0.25 μl/h of 3.33 mg/ml recombinant murine leptin) and the other half were infused with PBS. Four from each group (two males and two females) were euthanized after 4 weeks treatment. Osmotic pumps were replaced in the rest and the mice then treated for a total period of 8 weeks. Wild-type littermates were also treated with leptin under high or low diet regimens. The animal protocol was reviewed and approved by the Institutional Animal Core and Use Committee (IACUC) at the Columbia University Medical Center.

For other subjects, including humans, recombinant leptin products can be prepared for use in the methods of the described invention by various methods. One such method is described in U.S. Pat. No. 6,001,968, the contents of which are incorporated by reference herein. Leptin includes, but is not limited to, recombinant human leptin (PEG-0B, Hoffman La Roche) and recombinant methionyl human leptin (Amgen). Leptin derivatives, e.g., truncated forms of leptin (see para. 33 above), useful in the described invention include: U.S. Pat. Nos. 5,552,524; 5,552,523; 5,552,522; 5,521,283; and PCT International Publication Nos. WO 96/23513; WO 96/23514; WO 96/23515; WO 96/23516; WO 96/23517; WO 96/23518; WO 96/23519; and WO 96/23520, the contents of which are incorporated by reference herein. Also leptin fusion products useful in the described invention include, but are not limited to, fc-leptin, which is a fusion peptide derived from leptin and the Fc immunoglobulin region (see U.S. Pat. No. 6,936,439 and U.S. Published Patent Application No. 20050163799, the contents of which are incorporated by reference herein). The terms “fusion protein” or “fusion product” as used herein refers to a protein created through genetic engineering from two or more proteins/peptides by creating a fusion gene (i.e., removing the stop codon from the DNA sequence of the first protein and appending the DNA sequence of the second protein in frame) so that the DNA sequence encoding the two or more proteins/peptides is expressed by a cell as a single protein.

Lipid Insults.

Time course studies were performed to determine the effects of the lipid insults (ceramide, cholesterol and linoleic acid) at 1, 4, 6 and 18 hours on cell viability, tau phosphorylation and Aβ production (data not shown). These studies identified optimal time points in which the lipids induced significant changes in the cellular readout compared to baseline. For cell viability and Aβ production, 18 h lipid exposure produced a maximal effect. For tau phosphorylation, 6 h lipid exposure produced a maximal effect.

Cell Viability Assay.

RA-SY5Y, at 2×10⁴ cells/well, were seeded in 96-well microplates and treated for 18 h with a range of concentrations of cholesterol to determine effective doses for 50-70% viability (data not shown), or in the presence of a range of concentrations of full length leptin or leptin fragments to determine effective doses to prevent cell death. Viability was assessed using the Cell-Titer Blue Viability Assay (Promega) by adding 20 μL of the reagent to each well for 4 h, and plates read by a microplate reader with fluorescence capabilities at EX₅₃₀₋₅₇₀ₙₘ/EM₅₈₀₋₆₂₀ₙₘ. Viability was determined using a standard curve of known cell number and plotted as a percent of non-treated or vehicle control.

Preparation of Cell Lysates.

RA-SY5Y were treated with leptin (100 ng/ml) or leptin fragments (25 and 100 nM) in the presence of ceramide (25 μM), or linoleic acid (30 μg/mL) for 6 h, and then harvested by scraping. Preparation of whole cell lysates from cell pellets was performed as described previously (Greco, S. J. et al., Biochem Biophys Res Comm 376 (2008) 536-541).

ELISAs.

Aβ₍₁₋₄₀₎ levels in cell culture media from SY5Y_{APP751} treated for 18 h with the aforementioned metabolic insults in the presence or absence of leptin, were determined using the human amyloidβ 1-40 ELISA kit (Invitrogen; Carlsbad, Calif.), and phospho- and total tau levels in RA-SY5Y lysates were determined using the human Tau pSer³⁹⁶, pThr²³¹ and total tau ELISA kits (Invitrogen) according to manufacturer's specific instructions. Aβ₍₁₋₄₀₎, phosho- and total tau levels were calculated from a standard curve developed with OD at 450 nm using 8 serial dilutions of known concentration.

Statistical Analysis.

All values are the mean±s.e.m. Variations between pairs of groups was evaluated with t-test and differences were considered significant when p<0.05. Statistical data tests were performed by using analysis of variance and Tukey-Kramer multiple comparisons with p<0.05 considered statistically significant.

### Example 1

### The Effects of Leptin on Aβ Production In Vitro

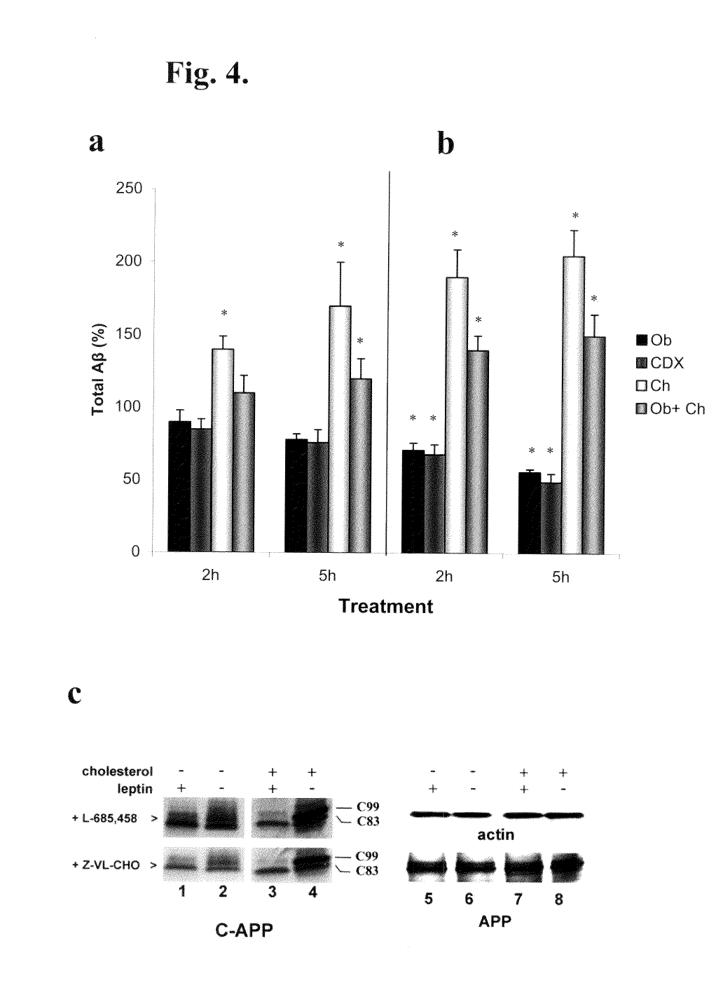

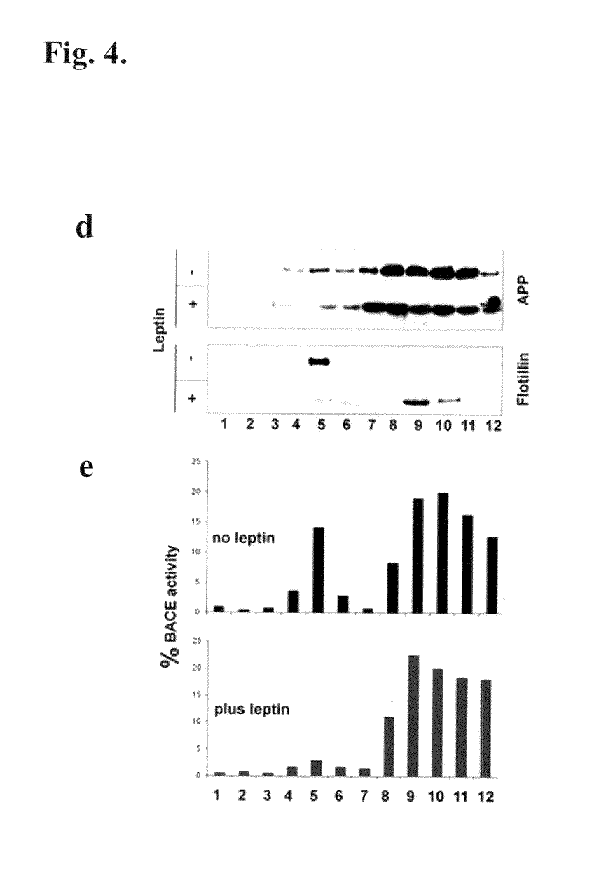

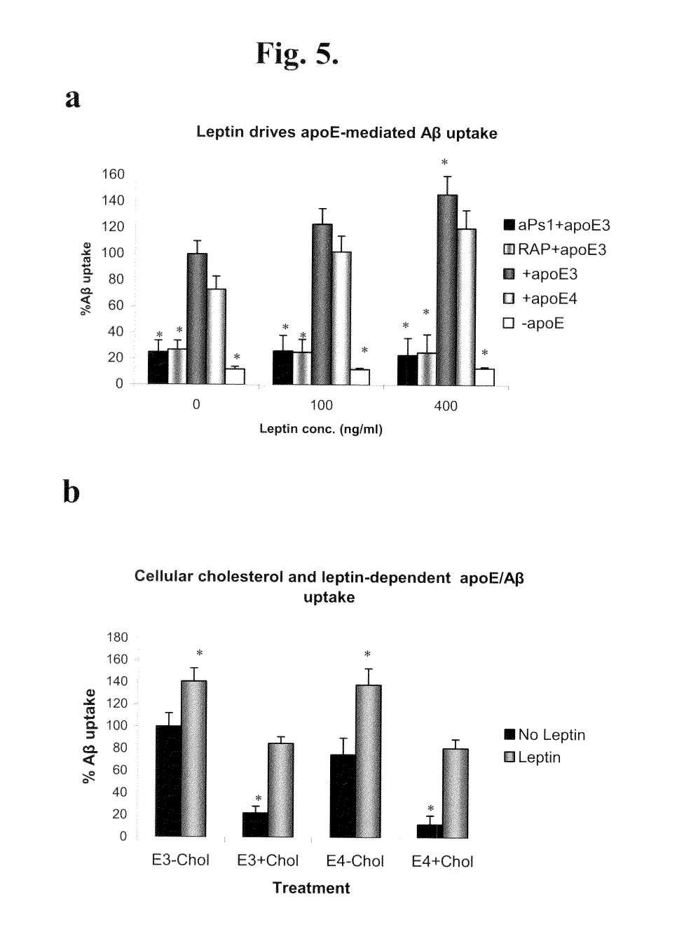

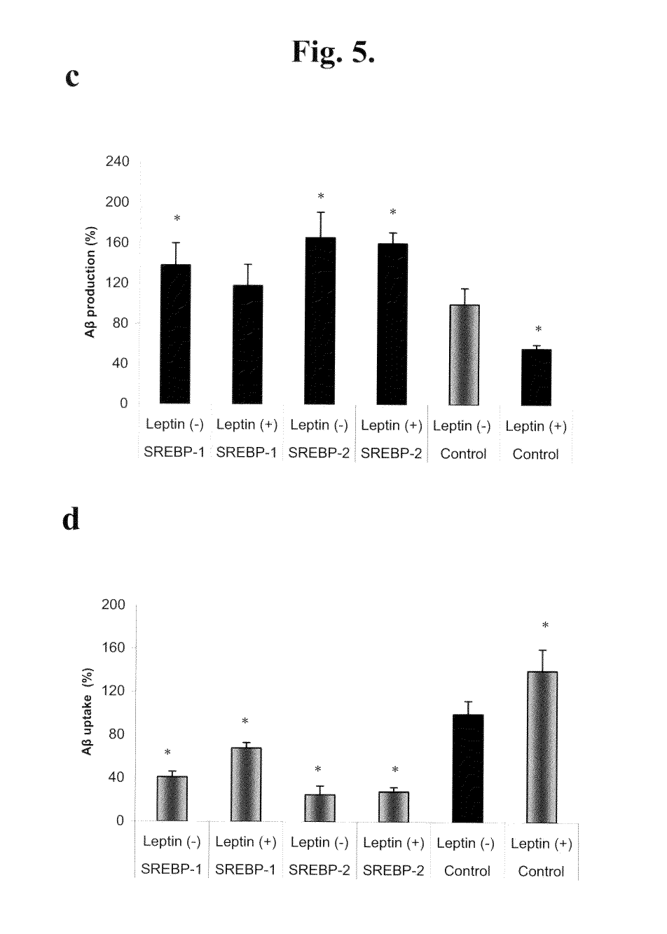

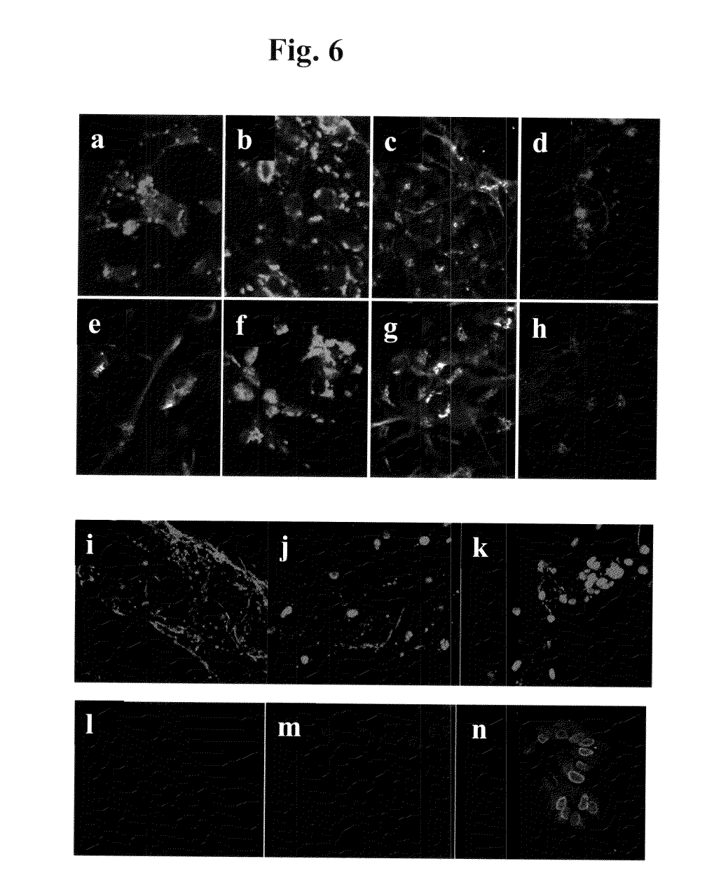

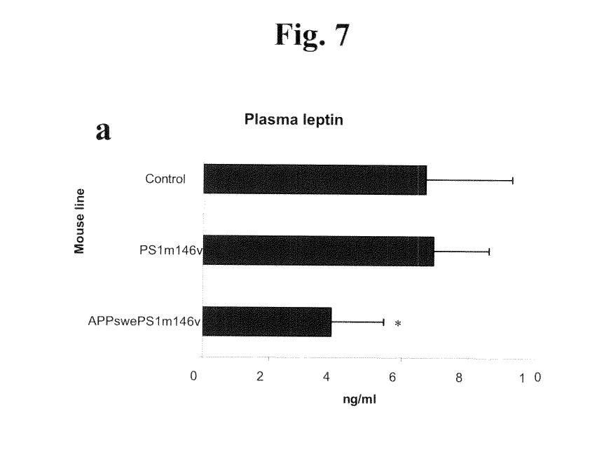

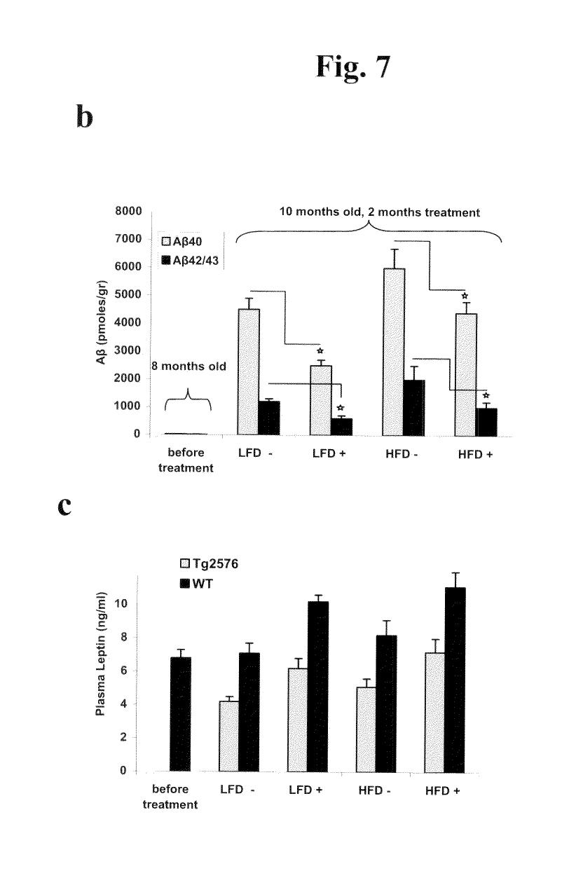

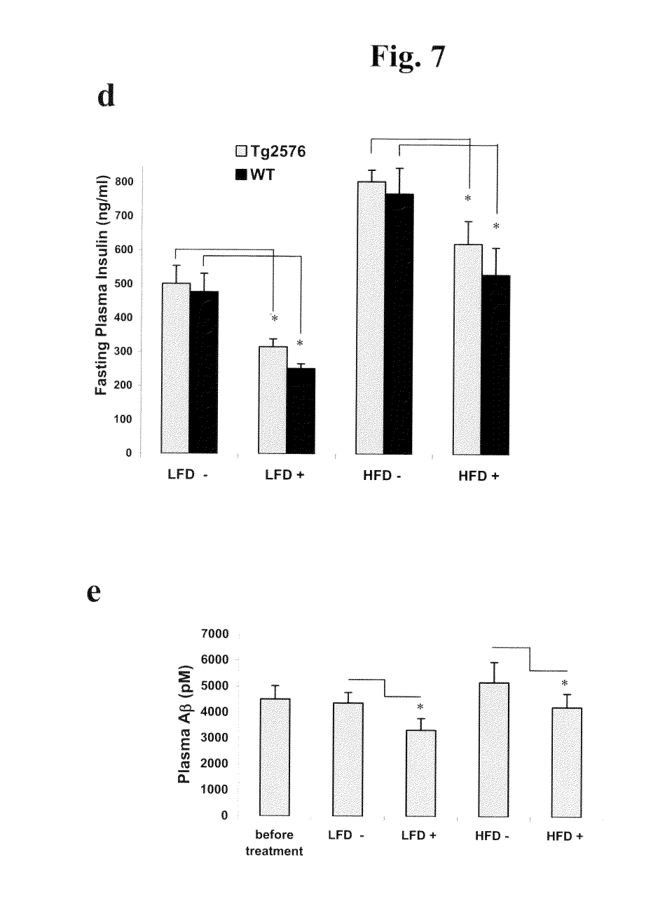

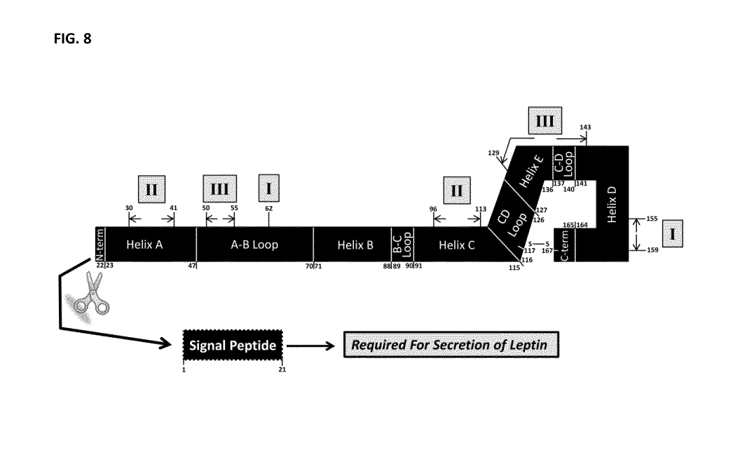

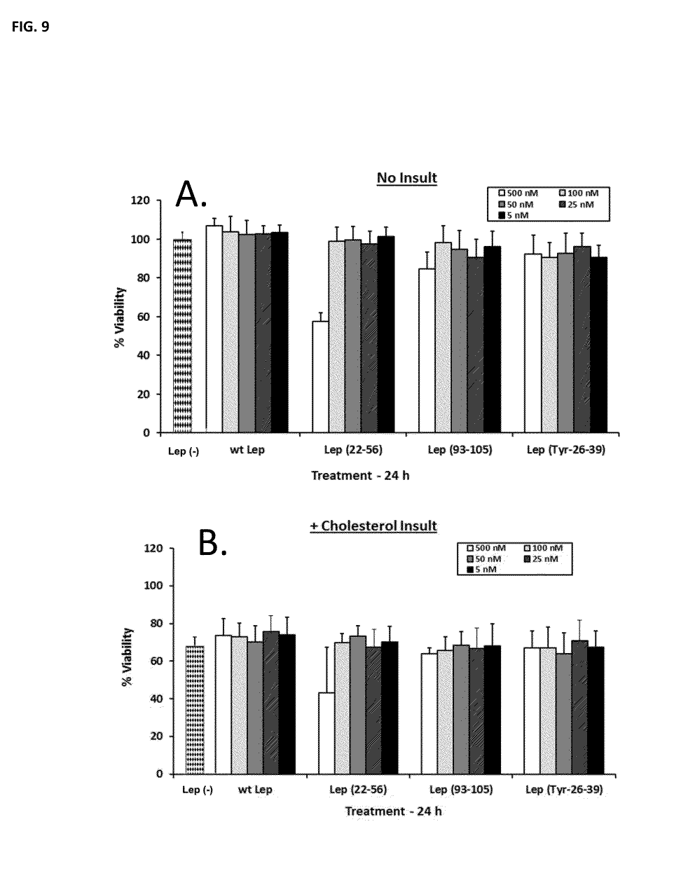

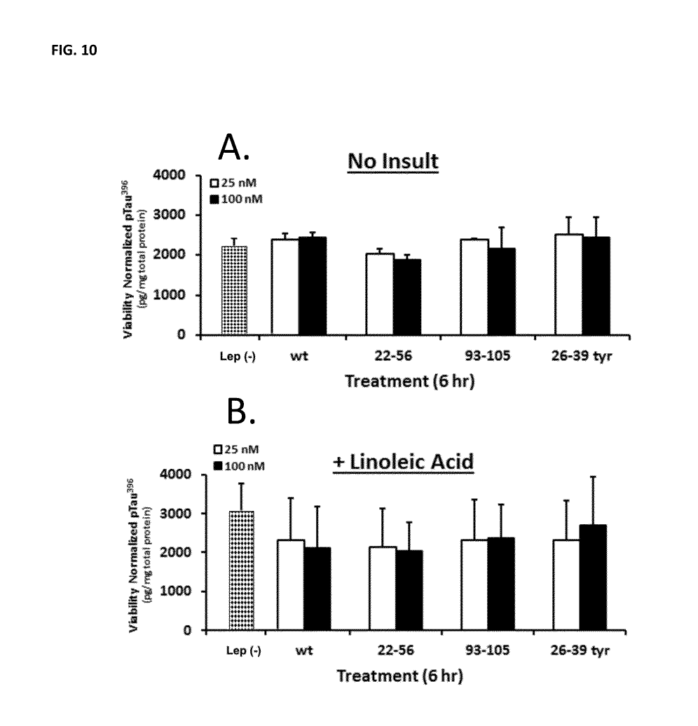

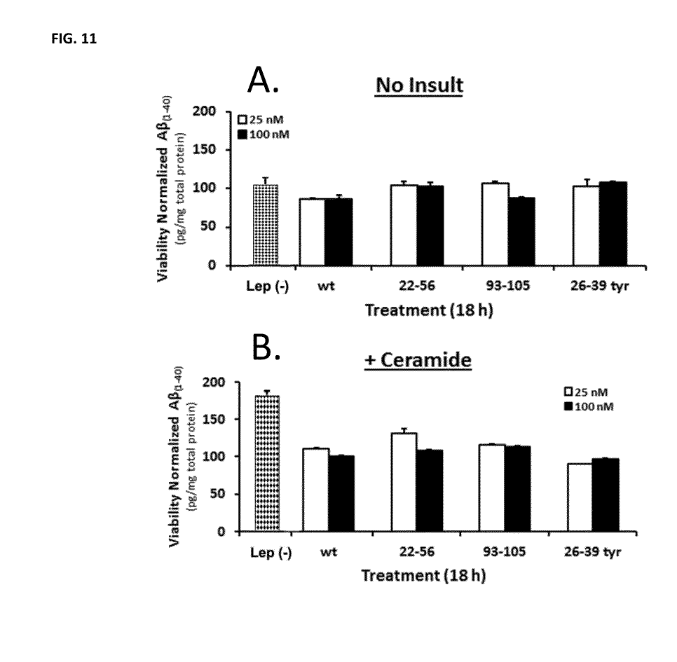

Human (SY5Y) or mouse neuroblastoma cell-lines (Neuro2a) commonly are used to study amyloid 13 metabolism in vitro. Neuro2a cells are stably transfected with hyg-sal 34 (SEQ ID NO: 11), a plasmid driving the expression of a recombinant fusion protein containing the human C-terminal fragment of APP of about 134 amino acids, CAPP₁₃₄ (SEQ ID NO: 10), Here, 5Y5Y or Neuro2a cultures were treated for 2 or 5 h with about 100 ng/ml or about 400 ng/ml leptin (FIG. 4a, 4b) Similarly, primary neurons from embryonic rat brain, infected with an adenovirus to direct the expression of CAPP 134 (SEQ ID NO:10) also were treated according to the same regimen.

FIG. 4 shows that leptin affects Af3 production through BACE in rafts. In panel (a), Neuro2a cells stably transfected with hyg-sa134 were treated for about 2 h or about 5 h with about 100 ng/ml leptin, Ob (black); about 125 mg/ml cyclodextrin, CDX (striped gray); about 5 mg/ml cholesterol, Ch (pale gray); and leptin plus cholesterol, Ob+Ch (medium gray). Media were collected and assayed for total Ala by ELISAs (Figueiredo-Pereira et al., J Neurochem. 72:1417-22 (1999)). Results are expressed as a percentage of the corresponding controls that did not receive drug treatment, measured at about 2 h and about 5 h respectively. Water soluble cholesterol (Sigma-Aldrich, MO) is a solution made of cholesterol balanced with CDX (40 mg cholesterol per gr CDX). In panel (b), Neuro2a cells stably transfected with hyg-sa134 were treated for about 2 h or 5 h with about 400 ng/ml leptin, Ob (black); about 250 mg/ml cyclodextrin, CDX (striped gray), about 10 mg/ml cholesterol, Ch (pale gray) and leptin plus cholesterol, Ob+Ch (medium gray) were used. In panel (c), SY5Y cells in culture were treated with about 400 ng/ml leptin or about 10 tg/ml cholesterol, or both, in the presence of the y secretase inhibitors L-685,458 (100 nM) or Z-VL-CHO (10011M) for about 5 h. Extracts prepared from harvested cells were analysed by SDS-PAGE and Western blotting using an antibody directed against the C-terminal fraction of APP (C-APP, lanes 1-4) or actin (top lanes 5-8) or full-length APP (bottom lanes 5-8). Immunoreactive bands C99 and C83 correspond to β- and γ secretase-generated fragments. In panel (d), extracts from SY5Y cells treated with and without leptin as above were solubilized in the presence of Triton X-100 and the insoluble fraction was applied to a discontinuous sucrose gradient as described (Cordy et al., 2003). Fractions collected from the bottom of the gradient were analysed by SDS-PAGE and Western blotting for the detection of APP and flotillin (marker for lipid rafts). A shift of the flotillin peak to more dense fractions of the gradient is observed following leptin treatment. In panel (e), fractions collected as above were assayed for P-secretase activity using a fluorescence-quenching assay (QTL Biosystems, NM). The results are expressed as the percent distribution of BACE activity within the gradient derived from cell cultures in the absence (black) or presence (gray) of leptin in the medium. Asterisks indicate that value is significantly different to that of the corresponding control (set at p<0.05).

Leptin caused a dose- and time-dependent decrease in the levels of Aβ detected in the media of transfected Neuro2a cells (56±5% following 5 h treatment with about 400 ng/ml leptin, FIG. 4b). Leptin was almost as efficient as methyl-P-cyclodextrin in lowering Aβ (FIG. 4a, 4b). In agreement with published data (Refolo et al., Neurobiol Dis. 7:321-31 (2000)), inclusion of water-soluble cholesterol in the culture media increased AP production (205±6% after 5 h with 10 1.1, M, FIG. 4b). Leptin partially reduced the amyloidogenic potency of cholesterol when co-administered with cholesterol (150±4% after 5 h with the highest concentrations of leptin, FIG. 4b). When ¹²⁵I-Aβ was included in the media during treatments in the presence or absence of 1 mM 1,10-phenanthroline, a general metalloprotease inhibitor which effectively inhibits degradation of secreted AP in vitro (Qiu et al., J Biol Chem. 272:6641-6646 (1997)), none of these treatments caused any significant differences in the degradation of AP in the medium, as assessed by measuring the percentage of ¹²⁵1-Aβ converted to TCA soluble radioactivity. Treatment with 1,10-phenanthroline did not cause any significant difference in the tracer's uptake by the cells (see below).

Two approaches were used to investigate whether the observed changes in Aβ production were concomitant with fluctuations in P-secretase activity. First, cultures were treated in the presence of the γ-secretase inhibitors L-685,458 (Sigma, 100 nM) or Z-VL-CHO (Figueiredo-Pereira, M. E. (Figueiredo-Pereira et al., J. Neurochem. 72:1417-22 (1999)), (100 μM) to allow the accumulation of 10 kDa CAPPβ (C99) (SEQ ID NO: 8) and 8 kDa CAPPα (C83) (SEQ ID NO: 16), the C-terminal fragments of APP generated by β- and α-secretase respectively. Under those conditions, 5 h treatment with 10 μM cholesterol caused an increase in C99 (SEQ ID NO: 8) but not C83 (SEQ ID NO: 16) (FIG. 4c, lanes #2, 4), consistent with an increase in β-secretase activity. This increase was abolished in the presence of 400 ng/ml leptin (FIG. 4c, lane #3, 4). In addition, APP levels as detected by Western blotting were unchanged and ³⁵S-Met metabolic labeling confirmed that neither APP synthesis (FIG. 4c, bottom lanes #5-8) nor proliferation, as detected by actin Western blots (FIG. 4c, top lanes #5-8), was affected. Leptin's effect on C99 (SEQ ID NO: 8) levels through possible inhibition of β-secretase also was observed in the absence of cholesterol (FIG. 4c, lanes #1, 2).

Second, activity of the beta-site amyloid precursor protein-cleaving enzyme (BACE) was measured in fractionated cell extracts using a fluorescence quenching assay (QTL Biosystems, NM) (FIG. 4e). LRs were prepared from a Triton X-100-insoluble membrane fraction further resolved by separation on a discontinuous sucrose gradient. All steps were carried out at 4° C. Confluent cells were scraped into 2 ml Mes-buffered saline (MBS, 25 mM Mes, 0.15M NaCl, pH 6.5) containing 1% (vol/vol) Triton X-100 and resuspended by passing them 5 times through a 25-gauge needle. An equal volume of 90% (wt/vol) sucrose in MBS then was added. Aliquots (1 ml) were placed in 5-ml ultracentrifuge tubes, and 4-ml discontinuous sucrose gradients consisting of 35% (wt/vol) sucrose in MBS (2 ml) and 5% (wt/vol) sucrose in MBS (2 ml) were layered on top. The sucrose gradients were centrifuged at 100,000×g for 18 h at 4° C. in a Beckman SW55 rotor, and fractions (0.5 ml) subsequently were harvested from the top to the bottom of the tube. (Cordy et al., Proc Natl Acad Sci USA. 100:11735-11740 (2003)).

In agreement with others (Id.), BACE activity in extracts from control cells was detected in a low density fraction also containing flotillin (FIG. 4d), an integral membrane protein known to be a marker for neuronal LRs (Bickel et al., J Biol Chem. 272:13793-802 (1997)). Noticeably, the bulk of BACE activity was detected outside LRs, at higher density fractions. In addition, the distribution of APP immunoreactivity, as detected by Western blotting, was very similar to that of BACE activity in gradient fractions. Only a small fraction co-migrated with the flotillin peak (FIG. 4d). Leptin treatment resulted in a subtle change of the composition and/or density of LRs, as determined by the distribution of BACE activity, APP and flotillin on sucrose gradient fractions. Flotillin migrated at heavier subcellular fractions as compared to controls, and the activity of BACE in the low density fractions was almost absent. A similar shift in the elution position for both flotillin and BACE was observed when cells were treated with CDX (not shown). These data are consistent with the notion that a prerequisite for BACE to generate Afi from APP is its association within LRs, and that the disruption of the lipid composition of those structures by leptin is sufficient to block the activity, presumably by hindering BACE's encounter with the substrate.

FIG. 6 shows that leptin can modulate free cholesterol-rich membrane domains and surplus cholesterol may trigger local leptin production. Neural cultures from E15 rat cerebral cortex were processed for enrichment of neurons (a-d) or astrocytes (e-h) as described (Takeshima et al., J. Neurosci. Methods 67: 27-41 (1996)). After about 7 days to about 10 days in culture, cultures were treated for about 5 h with about 10 pg/ml cholesterol (b, f) or about 400 ng/ml leptin plus cholesterol (c, g) or leptin alone (d, h). Controls (a, e) were treated with culture media alone. Filipin staining was performed as described (Feng et al., Nat. Cell Biol. 5: 781-92 (2003)). Neurons (i-k) and astrocytes (l-n) prepared as above were treated with 0 μM (i, 1), 5 μM (j, m) or 10 μM cholesterol (k, n) for 5 h. Immunostaining was performed for leptin (A.F.Parlow, Harbor-UCLA, CA).

In agreement with its ability to modulate the lipid composition of membranes, leptin treatment of primary neurons (FIG. 6a-d) and astrocytes (FIG. 6e-h) diminished filipin labeling (FIGS. 6d and 6h). Filipin is a fluorescent polyene antibiotic that binds to plasma membrane cholesterol (Feng et al., Nat Cell Biol. 5:781-92 (2003)). Further, the presence of leptin in cultures prohibited an increase in filipin labelling by cholesterol (FIGS. 6b and 60 in both cell types (FIGS. 6c and 6g).

Leptin's ability to lower the production of AP was mimicked by (a) 5-(tetraecyloxy)-2-furoic acid (TOFA), a long chain fatty acid inhibitor of fatty acid synthesis that blocks the synthesis of malonyl-CoA by acetyl CoA carboxylase (“ACC”) (Kempen et al. J Lipid Res. 36:1796-1806 (1995)) and (b) cerulenin, an irreversible fatty acid synthase (“FAS”) inhibitor (Loftus et al., Science. 288:2379-81 (2000); Mobbs, Science. 288:2379-81 (2002)). In contrast, etomoxir (ethyl-2-[6-4-chlorophenoxy)hexyl)oxirane-2-carboxylate), an inhibitor of fatty acid oxidation at the level of carinitine palmitoyl transferase 1 (CPT1) (Minokoshi et al., 2002. Nature. 415:339-43 (2002)), increased AP production (Table 1). This is consistent with an association between leptin's prolipolytic/antilipogenic properties and APP metabolism. Similar results were obtained with SY5Y cells and adenovirus vector-infected primary neurons derived from embryonic rat brains (Table 1). These findings confirm that metabolic pathways involving neuronal lipids and their distribution in membrane compartments influence Aβ production and establish that these can be controlled partially by exogenous leptin.

As Aβ homeostasis and lipid homeostasis are both the result of their production and clearance/uptake, respectively, the effect of leptin on the uptake of extracellular Aβ by SY5Y cells in culture also was investigated. It has been demonstrated that this process is facilitated by apolipoprotein E (“apoE”), which binds to Aβ and directs its capture via the Low-Density Lipoprotein Receptor Related Protein (“LRP”) and the subsequent endocytosis/degradation of the protein-lipid complex by endosomes/lysosomes where only LRP is recycled. Without being limited by theory, this may be the primary mechanism by which neurons absorb lipids from circulating high-density lipoprotein-(HDL)-like lipoproteins from the brain interstitial space (Danik et al., Crit Rev Neurobiol. 13:357-407 (1999)). For the purpose of these experiments, however, lipid-poor apoE was utilized (Narita, J Biochem. 132:743-749 (2002)).

FIG. 5 shows that leptin affects apoE-dependent An-uptake and the possible involvement of SREBPs. In panel (a), Aβ uptake was measured in SY5Y cells following their treatment at 0 ng/ml, 100 ng/ml or 400 ng/ml leptin. Uptake also was measured in cells previously transfected with antisense DNA for PS1 as described (Tezapsidis et al., FASEB 117:1322-1324 (2003)) (black). Uptake is expressed as the percentage of that observed with Aβ pre-incubated with apoE3 (medium gray) in the absence of leptin (first set of columns). Inclusion of RAP (gray stripe) and omission of apoE (white) abolished uptake. Leptin induced a dose-dependent increase in Aβ uptake with a preference for apoE3 (medium gray) over apoE4 (light gray). In panel (b), SY5Y cells were pre-treated with 10 mg/ml cholesterol (+Chol) or normal medium (−Chol). Then Aβ uptake was measured following its preincubation with apoE3 (E3) or apoE4 (E4) in the absence (black) or the presence (gray) of about 400 ng/ml leptin. Cells were more resistant to taking-up Aβ when loaded with cholesterol. Asterisks indicate that the value is significantly different to that set as 100% (set at p<0.05). In panel (c), SY5Y cells were transiently transfected with SREBP-1 or SREBP-2 cDNA or an empty vector (Control). Then Aβ was measured in the medium by ELISAs (Figueiredo-Pereira et al., J Neurochem. 72:1417-22 (1999)) following treatment with (+) or without (−) leptin. Results are expressed as the percentage of the Aβ produced in cells transfected with empty vector that did not receive leptin treatment, set at 100% (grey bar). In panel (d), Aβ uptake was measured in SY5Y cells prepared as in panel (c). Uptake was performed using Aβ/apoE3 complexes. Results are expressed as the percentage of the Aβ taken-up by cells transfected with empty vector that did not receive leptin treatment, set at 100% (black bar).

Leptin increased the uptake of apoE-Aβ in a dose-dependent fashion (FIG. 5a, striped and white bars for apoE3 and apoE4, respectively). Interestingly, the E3 allele of apoE was more efficient in delivering Aβ to the cell than the E4 allele. This indicates that the apoE isoform associated with increased risk for AD may be more resistant to the beneficial action of leptin in promoting lipid delivery to neurons and degradation of Aβ. Next, SY5Y cells were preloaded with cholesterol by introducing a preincubation step with cholesterol/CDX, and compared to controls preincubated with medium. Only 22±6% of apoE3-AP was taken up by cholesterol-loaded SY5Y cells compared to controls (FIG. 5b, black bars, first two pairs).

TABLE 1
The effect of metabolic regulators on AP production by transfected Neuro2a cells,
SY5Y cells or primary embryonic rat neurons infected with adenovirus.

| | | Neuro2a/ | | Neurons/ |
|---|---|---|---|---|
| | | SEAP-APP | SY5Y | SEAPPAPP |
| Inhibitor or | Target or | Aβ (% | AD (% | AD (% |
| Agent | Action | control) | control) | control) |
| TOFA, 200 μM | ACC | 40 ± 15 | 58 ± 12 | 35 ± 4 |
| Cerulenin, 200 μM | FAS | 52 ± 12 | 65 ± 9, NS | 66 ± 5 |
| Etomoxir, 40 μM | CPT-1 | 154 ± 14 | 142 ± 14 | 158 ± 14 |
| Peptide YY (3-36), | Anti-obesity | 92 ± 9, NS | 96 ± 7, NS | 98 ± 5, NS |
| 25 μM | | | | |
| Ciliary neurotrophic | Anti-obesity, | 95 ± 4, NS | 96 ± 8, NS | 89 ± 12, NS |
| factor, 25 μM | neurotrophin | | | |
| Leptin, 400 ng/ml | Anti-obesity, | 56 ± 5 | 38 ± 7 | 35 ± 4 |
| | Energy balance, | | | |
| | immunomodulation | | | |

Results are expressed: as mean ± SEM from 4 experiments, each with 3 determinations. Values are expressed as a percentage of total Aβ found in the conditioned media of cells not receiving treatment. In 5 h SY5Y cells produced 252 ± 50 pM, Neuro2a-SEAP-APP produced 820 ± 210 pM and Neurons/SEAPP-APP produced 131 ± 83 pM. Student's t test was used and statistical significance was set at p50.05. NS: statistically non-significant; TOFA: 5-(tetradecyloxy)-2-furancarboxylic acid; ACC: Acetyl CoA carboxylase; FAS: Fatty acid synthase; CPT-1: carnitine palmitoyl transferase-1

Addition of about 400 ng/ml leptin during the cholesterol pre-incubation period and during the uptake almost completely reversed the phenotype of these cells to that of controls (FIG. 5b, striped bars with leptin, black bars without leptin). These results suggest that leptin increases the capacity of neurons to take-up apoE-Aβ (and presumably lipids) which may be of paramount importance under conditions of remodelling and/or repair. LRP-mediated apoE-lipoprotein internalization is arbitrated through clathrin-coated pits, suggesting that Aβ uptake may not involve membrane microdomains. However, there is increased awareness that LRs and clathrincoated pits may not be exclusive concepts.

To gain insight into the specificity of leptin's ability to modulate Aβ production, cells were treated for 5 h with peptide YY (3-36), a gut-derived hormone affecting daily food intake that is believed to influence hypothalamic circuits (Batterham et al., N Engl J Med. 349:941-8 (2003)) and Ciliary Neurotrophic Factor (CNTF), a member of the gp130 family of cytokines that can regulate survival and differentiation of many types of developing and adult neurons (Sleeman et al., Pharm Acta Hely. 74:265-72 (2000)). At equimolar concentrations (25 μM) neither peptide changed AO′ production in a statistically significant way (Table 1), and this also was observed at higher (150 μM) concentrations (not shown).

To date, three SREBP isoforms, SREBP-1a (SEQ ID NO:17), SREBP-1c (SEQ ID NO:18) and SREBP-2 (SEQ ID NO:19) are known. Two isoforms, SREBP-1a and SREBP 1c, are transcribed from the SREBP-1 gene by alternative (or multiple) promoter usage for the same gene. The acidic transactivation domain that mediates interactions with chromatin modifying coactivators is shorter in SREBP-1c. As a result, SREBP-1c is a weaker transcriptional activator than SREBP-1a (Shimano et al. J. Cli. Inv. 99 (1997) 846-854). As used herein, the term SREBP-1 refers to the a isoform of SREBP-1. SREBP-2 (SEQ ID NO: 19) is more selective in activating the transcription of cholesterol biosynthetic genes, whereas SREBP 1 (SEQ ID NO: 17) and SREBP-1c (SEQ ID NO: 18) preferentially regulate fatty acid synthesis, however there is considerable overlap in their transcriptional activity.

The term “transactivation” as used herein refers to a technique used in molecular biology to control gene expression by stimulating transcription. It can be used to turn genes on and off. During transactivation, the transactivation gene and special promoters of DNA are inserted into the genome at areas of interest. The transactivator gene expresses a transcription factor that binds to specific promoter region(s) of DNA, causing that gene to be expressed. The expression of one transactivator gene can activate multiple genes, as long as they have the specific promoter region attached.

The term “coactivators” as used herein refers to a diverse array of gene regulatory proteins that do not themselves bind DNA but assemble on DNA-bound gene regulatory protein. They connect sequence-specific DNA binding activators to the general transcriptional machinery or help activators and the transcriptional apparatus navigate through the constraints of chromatin. Coactivator functions can be broadly divide into two classes: (a) adaptors that direct activator recruitment of the transcriptional apparatus, (b) chromatin-remodeling or -modifying enzymes.

It was of interest that SREBP-1c (SEQ ID NO: 18) mRNA and protein have been shown to be increased in the ob/ob mouse (Shimomura et al., J Biol Chem. 274:30028-32 (1999)), suggesting that leptin could regulate SREBP-1c (SEQ ID NO: 18) levels. To test this, SY5Y cells were transfected with modified pcDNA3.1 vectors to drive the expression of SREBP-1 (SEQ ID NO: 17) or SREBP-2 (SEQ ID NO: 19) under the CMV promoter, and some of the experiments of Aβ production or uptake in the presence or absence of leptin as already described were repeated.

As shown in FIG. 5, SREBP-2 (SEQ ID NO: 19) transfected cells were more resistant to the inhibition of AP production by leptin as compared to SREBP-1 (SEQ ID NO: 17) transfected cells (FIG. 5c). In addition, SREBP-2 (SEQ ID NO: 19) cells were resistant to the increase of apoE/Aβ uptake by leptin (FIG. 5d). Noticeably, transient expression of SREBP-1 (SEQ ID NO: 17) increased the production of Aβ to 138±22% as compared to controls (FIG. 5c) and reduced the uptake of apoE/Aβ to 41±5% as compared to controls (FIG. 5d). SREBP-2 (SEQ ID NO: 19) expression increased production of Aβ to 166±25% and inhibited uptake of apoE/Aβ to 25±8%. Without being limited by theory, at least two different scenarios could explain these results: a) leptin limits the availability of a common precursor for fatty acids and cholesterol (i.e. acetyl-CoA) or b) post-leptin receptor signaling events somehow turn-off SREBP-1 (SEQ ID NO: 17), causing a reduction in cholesterol, which is important for Aβ turnover. While the minor changes observed in SREBP-1 (SEQ ID NO: 17) transfected cells in the presence of leptin support the second possibility, both may be working in cohort.

In agreement with previous reports (Ur et al., Neuroendocrinology. 75:264-72 (2002)) leptin was detected immunocytochemically in dispersed neural cultures prepared from rat embryonic brain (FIG. 6i-6n) and by Western blotting of extracts of these cultures (data not shown). Similarly, the leptin receptor was detected in these cultures (not shown) (Couce et al., Neuroendocrinology. 66:145-50 (1997)). Interestingly, cholesterol treatment enhanced the levels of leptin-like immunoreactivity in both neurons (FIG. 6i-6k) and astrocytes (FIG. 6l-6n) in a dose dependent-fashion. Without being limited by theory, leptin appears to serve as a local feedback signal to inhibit further cholesterol synthesis and uptake, which in turn has an impact on Ap production and uptake. Consequently, deficiencies in either leptin or transduction of its signal in neural cells could be contributory to AD-related pathways. Within the CNS, glia are the cell group prominently synthesizing apoE, cholesterol and phospholipid rich HDL-like lipoprotein particles (Fagan et al., J Biol Chem. 274:30001-7 (1999)). (As used herein, the terms “glia” or “glial cell” are used interchangeably to refer to the connective tissue cells of the CNS that serve as the supportive structure that holds together and protects neurons). Lipids are required by neurons during plasticity-related neuritic arborization/outgrowth or during neural progenitor cell proliferation. (“Neural plasticity” refers to the ability of neural circuits to undergo changes in function or organization due to previous activity). Nonetheless, excess cholesterol and AP can be harmful. Without being limited by theory, bi-directional communication between neurons and glia, based on local leptin (rather than leptin derived from the circulation) and leptin signaling pathways, may serve to balance local lipid requirement. It has been demonstrated previously that leptin can modulate hippocampal excitability via activation of large conductance calcium-activated potassium ion channels (Shanley et al., Nat Neurosci. 5:299-300 (2002)), supporting a link between endocrine factors and AD.

### Example 2

### In Vivo Leptin Activity

Plasma leptin levels were measured in transgenic mice engineered to express mutations linked to familial AD: APP with the Swedish mutation (APP_{swe}) (SEQ ID NO: 13), PS1 with the M146V substitution (PS1_{M146V}) (SEQ ID NO: 15), and both APP_{swe} (SEQ ID NO: 13) and PS1_{M146V} (SEQ ID NO: 15). Among those, only the transgenic mice expressing APP_{swe} exhibit AD-like pathology. The APP_{swe}-expressing mice in the PS1_{M146V} background exhibit AD-like pathology at a younger age (6 months). The PS1_{M146V} mice do not develop AD-like pathology.

FIG. 7 shows a deficiency of leptin in AD transgenic mice and the effect of leptin supplementation on amyloid load. In panel (a), plasma leptin was quantified in one year old mice with the following genotypes: i) double mutant APP_{swe}/PS1_{M146V} ii) single mutant PS1_{M146V} and wild-type (a cross between C57BL/6Ntac and B6SJLF1). Asterisk indicates that value is significantly different to that of non-transgenic controls (set at p<0.05). Plasma Ail was also measured in these mice prior to treatment. Panel (b) shows Tg2576 mice under high fat (HFD) and low fat (LFD) diets one week prior to the implantation of the Alzet pump subcutaneously (s.c) for constant delivery of leptin (+) or vehicle PBS (−) at 8 months of age. The pump was replaced after 4 weeks. Formic acid extracts of brains obtained as described previously (Kawarabayashi et al., J Neurosci. 21:372-81 (2001)) were used to determine the Aβ40 (SEQ ID NO: 4) and Aβ42 (SEQ ID NO: 5) content by commercially available ELISA kits (KMI Diagnostics, MN), as described by the manufacturer. Only APP_{swe}-expressing mice (Tg2576) contained detectable amounts of A13 species. At 8 months of age the Tg2576 mouse has very low levels of Aβ. In panel (c), plasma leptin was determined by radioimmunoassay (“RIA”) (LINCO Research, Inc.) in 8 month old Tg2576 and WT littermate mice and then again following treatments as described in FIG. 4b. Leptin also was measured in WT but not Tg2576 mice prior to treatment. In panel (d), plasma insulin was determined by RIA (LINCO Research, Inc.) in 8 month old WT and Tg2576 mice and then again following a 2 month LFD or HFD with (+) or without (−) leptin infusion. In panel (e), plasma total Aβ (Aβ40 (SEQ ID NO: 4) plus Aβ42/43 (SEQ ID NO: 5/SEQ ID NO: 6) was measured in 8 month Tg2576 mice and then again following a 2 month LFD or HFD with (+) or without (−) leptin infusion.

In both males and females, circulating leptin levels were approximately half of those in littermates not expressing the APP_{swe} (SEQ ID NO: 13), regardless of the expression of PS1_{M146V} (SEQ ID NO: 15) (FIG. 7a and FIG. 7c).

Based on leptin's antiamyloidogenic activity in vitro as described above and the apparent leptin deficiency in the APP_{swe}-expressing mice, the effect of chronic peripheral administration of leptin to animals under a high or low fat diet was investigated (FIG. 7b-70. Constant subcutaneous (s.c.) infusion of murine leptin (0.25/h of 3.33 mg/ml) (or PBS as placebo) was administered to Tg2576 or wild-type (WT) littermate mice for up to 8 weeks from about 8 months of age under the two different dietary regimens described above in Methods. Brain Aβ levels of the APPs. hemizygous mouse rise between 6-9 months and lead to the appearance of the first thioflavin S positive amyloid plaques in the hippocampus and cerebral cortex, approximately 2 months later (Thioflavin S is a histologic stain used to demonstrate amyloid containing neurofibrillary tangles and senile plaques in diseased brain tissue sections). APPs_{we} expressing transgenic Tg2576 mice under the high fat diet had higher levels of both Aβ40 and Aβ42 in formic acid extracts of brain homogenates when compared to those under the low fat diet (FIG. 7c), in agreement with others (Refolo et al., Neurobiol Dis. 7:321-31 (2000)). Neuropathological examination was not performed because amyloid deposits in the form of cored or diffuse plaques in the 10 month-old Tg2576 brains are too few (Kawarabayashi et al., J Neurosci. 21:372-81 (2001)) to allow statistically significant correlative studies. Further, plasma leptin and insulin levels were measured.

The level of leptin was confirmed to be lower in APP_{swe}-expressing mice at 10 months, compared to controls, irrespective of diet and weight (FIG. 7b, 7d). In contrast, fasting insulin levels in mice of both genotypes fluctuated similarly and were elevated by high fat diet and lowered by low fat diet. Leptin treatment decreased fasting insulin levels in all groups, consistent with its ability to increase insulin sensitivity (FIG. 7d). Finally, quantification of total Aβ in the plasma (FIG. 7e) of the Tg2576 mouse revealed that leptin treatment was able to lower the levels of circulating AP under both diets. Without being limited by theory, it is not known whether this reflects the lowering of the CNS amyloid load shown in FIG. 7c, or is related to changes in peripheral Aβ production.

As the APP_{swe} transgene in the Tg2576 mouse is under the control of the Prion-protein promoter (Hsiao et al., Science. 274:99-102 (1996)), allowing its expression in the CNS and periphery (Ford et al., Neuroscience. 113:177-92. (2002); Lemaire-Vieille et al., Proc Natl Acad Sci USA. 97:5422-7 (2000)), and leptin is primarily produced in adipocytes, the adipose tissue extracted from these mice under high or low fat diets, plus or minus leptin treatment, was examined as described (Yu et al., J Biol Chem. 277:50876-84 (2002)). Higher levels of APP expression in adipocytes derived from the transgenic mice compared to expression in wild-type animals was detected. Leptin treatment had no apparent influence on this expression (data not shown). Interestingly, transgenic adipocytes were less responsive with regards to insulin-induced expression of leptin and glucose uptake than adipocytes from controls (data not shown). This was similar to the changes associated with senescence developed over time with normal aging in adipocytes (Yu and Zhu, J Biol Chem. 277:50876-84 (2003)).

### Example 3

### Effect of Metabolic Challenges on Neuronal Cell Viability

Doses of cholesterol which decreased neuronal viability by 25-50% (27.5 μg/mL) after 18 h treatment (data not shown) were used to determine whether co-treatment with full length or fragments of leptin at a range of concentrations could attenuate the toxic effects of the cholesterol (FIG. 9). RA-SY5Y were treated for 18 h with full length or fragments of leptin ranging from 5 nM (white bars) to 500 nM (black bars), or no leptin control (checkered bars) in the presence (FIG. 9B) or absence (FIG. 9A) of cholesterol, and cell viability measured. Cholesterol insult induced a decrease in cell viability in the range of 35±5% when treated without Leptin (9B; checkered bar). Full length and fragments of leptin showed no effect on cell viability in the absence of cholesterol (FIG. 9A), while full length and Lep (22-56) trended towards increased viability in the presence of cholesterol at all doses (FIG. 9B; second and third group of bars from left). These results demonstrated that the leptin fragments were well tolerated by the cells compared to the full length peptide. Based on these data we proceeded to analyze full length leptin and the fragments at doses of 25 and 100 nM in subsequent assays.

### Example 4

### AD-Related Pathway Activation Following Exposure to Exogenous Lipids

Metabolic stress has been linked to the contribution/activation of AD pathological pathways, with lipids known to play an important role (Puglielli, R. E. et al., Nat Neurosci 6 (2003) 345-351; Glockner, F. et al., Journal of Neuropathology and Experimental Neurology 70 (2011) 292-301; Wilson, D. M. et al., The American Journal of Pathology 150 (1997) 2181-2195). We therefore investigated the extent to which extracellular lipids can increase the phosphorylation of tau (FIG. 10) and Aβ production (FIG. 11) in neuronal cells, and determined whether full length leptin and leptin fragments could prevent these effects. RA-SY5Y were treated for 6 h with full length leptin or leptin fragments (25 or 100 nM) or no leptin control (checkered bars) in the presence (FIG. 10B) or absence (FIG. 10A) of linoleic acid, and phosphorylation of tau at pTau³⁹⁶ was measured by ELISA. Challenging cells with linoleic acid resulted in an approximately 50% increase in tau phosphorylation (FIG. 10B, checkered bar). Simultaneous treatment with full length or fragments of leptin showed limited effects on tau phosphorylation in the absence of linoleic acid (FIG. 10A), while all treatments trended towards decreased phosphorylation in the presence of linoleic at all doses (FIG. 10B; white and black bars). These results suggest that the leptin fragments span regions of the full length peptide that are functionally active in regulating tau phosphorylation.

In parallel to the tau studies, we investigated whether exposure of neuronal cells to high lipids could exacerbate the extracellular production of Aβ, and likewise determined whether full length leptin and leptin fragments could prevent these effects (FIG. 11). SY5Y stably over-expressing human APP₇₅₁ were treated for 18 h with leptin or leptin fragments (25 or 100 nM) or no leptin control in the presence or absence of ceramide, and the amount of extracellular Aβ₍₁₋₄₀₎ measured by ELISA. Ceramide induced an approximately 75% increase in the amount of Aβ₍₁₋₄₀₎ produced (FIG. 11B; checkered bar). Treatment with full length or fragments of leptin showed limited effects on Aβ₍₁₋₄₀₎ production in the absence of ceramide (FIG. 11A), while all treatments decreased Aβ₍₁₋₄₀₎ in the presence of ceramide at all doses (FIG. 11B; white and black bars). Full length leptin and leptin fragments' ability to reduce Aβ₍₁₋₄₀₎ production following ceramide challenge was similar to tau phosphorylation in that treatment reduced the readout to near baseline levels in the absence of the lipid insult. In summation, these data suggest that fragments of leptin spanning known functionally active domains are a plausible approach as an AD therapeutic.

Without being limited by theory, these studies support the conclusion that early leptin administration to Tg2576 mice has an impact on CNS amyloid deposition and should affect synaptic function and behavioral profile. These studies also demonstrate that a low fat diet in combination with leptin supplementation could be a potential palliative treatment for certain AD cases.

Without being limited by theory, the association between leptin/leptin signaling and AD-like pathobiology reported here in a mouse model is perhaps complementary, or works in parallel, to pathways involving insulin, as reviewed recently (Watson, CNS Drugs. 17:27-45 (2003)). Plasma leptin levels decrease with aging in a manner which is more profound in postmenopausal women (Isidori et al., The Journal of Clinical Endocrinology & Metabolism. 85:1954-1962 (2000)) and leptin receptors are present throughout the brain including the hippocampus and olfactory bulb, domains affected early during the course of the disease. Because dysregulation of pathways associated with leptin may play a critical role in the pathogenesis of AD, leptin treatment may be beneficial in some AD cases, specifically those experiencing weight loss and/or have low circulating leptin levels.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

The sequence listing in the form of a .txt file named “117468-010302 Sequence Listing_ST25.txt” containing SEQ ID NOs: 1-42, electronically filed on Apr. 4, 2012 as a substitute for the sequence listing originally filed with the application, is incorporated by reference herein.

What is claimed is:

1. A method for treating a progressive cognitive disease, cognitive disorder, or cognitive condition resulting from accumulation of an amyloid peptide, comprising:
administering to a subject in need thereof a first composition comprising
(i) a therapeutic amount of a leptin, a leptin mimic, or a pharmaceutically acceptable salt thereof, and
(ii) a pharmaceutically acceptable carrier,
wherein the leptin or the leptin mimic is a recombinant human leptin, a pegylated recombinant human leptin (PEG-OB), a recombinant human methionyl leptin, a leptin peptidomimetic, a biologically active fragment of leptin, a fusion peptide of leptin with an Fc fragment of immunoglobulin, a fusion peptide of the biologically-active fragment of leptin with the Fc fragment of immunoglobulin, a leptin agonist, or a combination thereof,
wherein the therapeutic amount of the leptin or the leptin mimic is effective to modulate accumulation of the amyloid peptide in brain.

2. The method according to claim 1, wherein the method further comprises monitoring circulating levels of the amyloid peptide.

3. The method according to claim 2, wherein the circulating levels of amyloid peptide are detected in a sample of cerebrospinal fluid or blood.

4. The method according to claim 1, wherein the method further comprises placing the subject on a low fat diet.

5. The method according to claim 1, wherein the progressive cognitive disease, cognitive disorder, or cognitive condition is a dementia, an amyloidosis, Down's syndrome, or cerebral amyloid angiopathy.

6. The method according to claim 5, wherein the progressive cognitive disease, cognitive disorder, or cognitive condition is Alzheimer's disease.

7. The method according to claim 5, wherein the progressive cognitive disease, cognitive disorder, or cognitive condition is senile systemic amyloidosis.

8. The method according to claim 5, wherein the progressive cognitive disease, cognitive disorder, or cognitive condition is cerebrovascular amyloidosis.

9. The method according to claim 1, wherein the amyloid peptide is an amyloid β (Aβ) peptide.

10. The method according to claim 1, wherein the first composition further comprises
(iii) a therapeutically effective amount of one or more lipolytic/antilipogenic compounds wherein the one or more lipolytic/antilipogenic compounds reduce amyloid β (Aβ) production, increase apoE-Aβ (Aβ) uptake, or both.

11. The method according to claim 1, wherein the first composition modulates accumulation of the amyloid peptide in the cerebral nervous system.

12. The method according to claim 1, wherein the first composition is administered by at least one route selected from the group consisting of orally, buccally, parenterally, intranasally, rectally, virally and topically.

13. The method according to claim 1, wherein the method further comprises serially administering a second composition comprising a therapeutically effective amount of one or more lipolytic/antilipogenic compounds, wherein the one or more lipolytic/antilipogenic compounds reduce amyloid β (Aβ) production, increase apoE-Aβ (Aβ) uptake, or both.

14. The method according to claim 13, wherein the method further comprises placing the subject on a low fat diet.

15. A method of improving resilience of cognitive function in a subject in need thereof, the method comprising:
(a) administering to the subject a composition comprising:
i. a cognitive function-enhancing or cognitive function stabilizing amount of leptin, a leptin mimic, or a pharmaceutically acceptable salt thereof,
wherein the leptin or the leptin mimic is a recombinant human leptin, a pegylated recombinant human leptin (PEG-OB), a recombinant human methionyl leptin, a leptin peptidomimetic, a biologically-active fragment of leptin, a fusion peptide of leptin with an Fc fragment of immunoglobulin, a fusion peptide of the biologically-active fragment of leptin with the Fc fragment of immunoglobulin, a leptin agonist, and a combination thereof, and
ii. a pharmaceutically acceptable carrier
wherein the cognitive function-enhancing or the cognitive function stabilizing amount of leptin or the leptin mimic is effective to modulate accumulation of an amyloid peptide in brain.

16. The method according to claim 15, wherein the composition is administered orally, buccally, parenterally, intranasally, rectally, virally or topically.

17. The method according to claim 15, further comprising:
(b) measuring the subject's ability to perform mental tasks.

18. The method according to claim 17, wherein the subject's ability to perform mental tasks is measured by at least one test for memory, computation, or attention.

19. The method according to claim 1, wherein the biologically active fragment of leptin comprises an amino acid sequence selected from the group consisting of SEQ ID NOs: 27, 28, 29, 30, 31, 32, 33, 34, 35, 36, 37, 38, 39, 40, 43, 44 and 45.

20. The method according to claim 1, wherein the biologically active fragment of leptin comprises a first and a second fragment,
wherein the first fragment has amino acid sequence SEQ ID NO: 41,
wherein the second fragment has amino acid sequence SEQ ID NO: 42, and
wherein the first fragment is covalently linked to the second fragment via a disulfide bond between cysteine at amino acid residue 96 of SEQ ID NO: 41 and cysteine at amino acid residue 8 of SEQ ID NO: 42.

21. The method according to claim 1, wherein the therapeutic amount is from about 0.01 mg per kg (of body weight) per day to about 0.5 mg per kg (of body weight) per day.

22. The method according to claim 1, wherein the subject in need thereof has a systemic leptin deficiency.

23. The method according to claim 22, wherein the composition restores, replenishes, or increases leptin levels.

24. The method according to claim 15, wherein the biologically active fragment of leptin has an amino acid sequence selected from the group consisting of SEQ ID NOs: 27, 28, 29, 30, 31, 32, 33, 34, 35, 36, 37, 38, 39, 40, 43, 44 and 45.

25. The method according to claim 15, wherein the biologically active fragment of leptin comprises a first and a second fragment,
wherein the first fragment has amino acid sequence SEQ ID NO: 41,
wherein the second fragment has amino acid sequence SEQ ID NO: 42, and
wherein the first fragment is covalently linked to the second fragment via a disulfide bond between cysteine at amino acid residue 96 of SEQ ID NO: 41 and cysteine at amino acid residue 8 of SEQ ID NO: 42.

26. The method according to claim 15, wherein the cognitive function-enhancing amount of the leptin, the leptin mimic, or the pharmaceutically acceptable salt thereof, reduces amyloid β (Aβ) production, increases apoE-Abeta (Aβ) uptake, or both.

27. The method according to claim 15, wherein the amyloid peptide is an amyloid β (Aβ) peptide.

28. The method according to claim 15, wherein the subject in need thereof has a systemic leptin deficiency.

29. The method according to claim 28, wherein the composition restores, replenishes, or increases leptin levels.

30. The method according to claim 15, wherein the cognitive function-enhancing amount is from about 0.01 mg per kg (of body weight) per day to about 0.5 mg per kg (of body weight) per day.

31. The method according to claim 15, wherein the composition further comprises
(iii) a therapeutically effective amount of one or more lipolytic/antilipogenic compounds, wherein the one or more lipolytic/antilipogenic compounds reduce amyloid β (Aβ) production, increase apoE-Aβ (Aβ) uptake, or both.

32. The method according to claim 15, wherein the method further comprises placing the subject on a low fat diet.

33. The method according to claim 15, wherein the method further comprises serially administering a second composition comprising a therapeutically effective amount of one or more lipolytic/antilipogenic compounds, wherein the one or more lipolytic/antilipogenic compounds reduce amyloid β (Aβ) production, increase apoE-Aβ (Aβ) uptake, or both.

34. A method for treating a progressive cognitive disease, cognitive disorder, or cognitive condition resulting from an increase in tau phosphorylation, comprising:
administering to a subject in need thereof a first composition comprising
(i) a therapeutic amount of a leptin, a leptin mimic, or a pharmaceutically acceptable salt thereof, and
(ii) a pharmaceutically acceptable carrier,
wherein the leptin or the leptin mimic is a recombinant human leptin, a pegylated recombinant human leptin (PEG-OB), a recombinant human methionyl leptin, a leptin peptidomimetic, a biologically active fragment of leptin, a fusion peptide of leptin with an Fc fragment of immunoglobulin, a fusion peptide of the biologically-active fragment of leptin with the Fc fragment of immunoglobulin, a leptin agonist, or a combination thereof,
wherein the therapeutic amount of the leptin or the leptin mimic is effective to decrease tau phosphorylation in brain.

35. The method according to claim 34, wherein the progressive cognitive disease, cognitive disorder, or cognitive condition is a dementia, an amyloidosis, Down's syndrome, or cerebral amyloid angiopathy.

36. The method according to claim 35, wherein the progressive cognitive disease, cognitive disorder, or cognitive condition is Alzheimer's disease.

37. The method according to claim 35, wherein the progressive cognitive disease, cognitive disorder, or cognitive condition is senile systemic amyloidosis.

38. The method according to claim 35, wherein the progressive cognitive disease, cognitive disorder, or cognitive condition is cerebrovascular amyloidosis.

39. The method according to claim 34, wherein the composition is administered orally, buccally, parenterally, intranasally, rectally, virally or topically.

40. The method according to claim 34, wherein the biologically active fragment of leptin comprises an amino acid sequence selected from the group consisting of SEQ ID NOs: 27, 28, 29, 30, 31, 32, 33, 34, 35, 36, 37, 38, 39, 40, 43, 44 and 45.

41. A method for increasing neuronal cell viability in a subject suffering from a progressive cognitive disease, cognitive disorder, or cognitive condition comprising:
administering to a subject in need thereof a first composition comprising
(i) a therapeutic amount of a leptin, a leptin mimic, or a pharmaceutically acceptable salt thereof, and
(ii) a pharmaceutically acceptable carrier,
wherein the leptin or the leptin mimic is a recombinant human leptin, a pegylated recombinant human leptin (PEG-OB), a recombinant human methionyl leptin, a leptin peptidomimetic, a biologically active fragment of leptin, a fusion peptide of leptin with an Fc fragment of immunoglobulin, a fusion peptide of the biologically-active fragment of leptin with the Fc fragment of immunoglobulin, a leptin agonist, or a combination thereof,
wherein the therapeutic amount of the leptin or the leptin mimic is effective to increase neuronal cell viability in the subject.

42. The method according to claim 41, wherein the progressive cognitive disease, cognitive disorder, or cognitive condition is a dementia, an amyloidosis, Down's syndrome, or cerebral amyloid angiopathy.

43. The method according to claim 42, wherein the progressive cognitive disease, cognitive disorder, or cognitive condition is Alzheimer's disease.

44. The method according to claim 42, wherein the progressive cognitive disease, cognitive disorder, or cognitive condition is senile systemic amyloidosis.

45. The method according to claim 42, wherein the progressive cognitive disease, cognitive disorder, or cognitive condition is cerebrovascular amyloidosis.

46. The method according to claim 41, wherein the composition is administered orally, buccally, parenterally, intranasally, rectally, virally or topically.

47. The method according to claim 41, wherein the biologically active fragment of leptin comprises an amino acid sequence selected from the group consisting of SEQ ID NOs: 27, 28, 29, 30, 31, 32, 33, 34, 35, 36, 37, 38, 39, 40, 43, 44 and 45.

48. A method for treating a progressive cognitive disease, cognitive disorder, or cognitive condition resulting from phosphorylation of tau in brain, comprising:
administering to a subject in need thereof a composition comprising
(i) a therapeutic amount of a biologically active fragment of leptin comprising an amino acid sequence selected from the group consisting of SEQ ID NOs: 43, 44 and 45, and
(ii) a pharmaceutically acceptable carrier,
wherein the therapeutic amount is effective to decrease phosphorylation of tau in brain.


SEQUENCE LISTING (45 sequences)

SEQ ID NO: 1 - protein, 770 aa, Homo sapiens
  MISC_FEATURE: Human APP, isoform a
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 751 aa, Homo sapiens
  MISC_FEATURE: Human APP, isoform b
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAIPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - protein, 695 aa, Homo sapiens
  MISC_FEATURE: Human APP, isoform c
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - protein, 40 aa, Unknown
  Mammalian
  MISC_FEATURE: Abeta40
DAEFRHDSGYEVHHQKLVFFAEDVGSNKGAIIGLMVGGVV

SEQ ID NO: 5 - protein, 42 aa, Unknown [sequence omitted]

SEQ ID NO: 6 - protein, 43 aa, Unknown
  Mammalian
  MISC_FEATURE: Abeta43
DAEFRHDSGYEVHHQKLVFFAEDVGSIIKGAIIGLMVGGVVIA

SEQ ID NO: 7 - protein, 671 aa, Unknown
  Mammalian
  MISC_FEATURE: sAPPbeta (based on SEQ ID NO: 1)
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKM

SEQ ID NO: 8 - protein, 99 aa, Unknown
  Mammalian
  MISC_FEATURE: 10 kda CAPPbeta (C99)
  MISC_FEATURE: 10 kDa CAPPbeta (C99)
DAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 9 - DNA, 1461 bp, Unknown
  Mammalian
  misc_feature: SEAP DNA sequence (corresponds to nucleotides 981-2441) of hyg-sa134 vector (SEQ ID NO: 11)
gttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgatgtgatcctaggtggaggccgaaagtacatgtttcgcatgggaaccccagaccctgagtacccagatgactacagccaaggtgggaccaggctggacgggaagaatctggtgcaggaatggctcggcgaacgccagggtgcccggtacgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcagacacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccttcatcgggctggccgctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttgg

SEQ ID NO: 10 - protein, 134 aa, Unknown
  Mammalian
  MISC_FEATURE: Amino acid sequence of CAPP134 (amino acid residues 630-770 of human APP, isoform a, SEQ ID NO: 1)
VEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 11 - DNA, 7506 bp, Unknown
  Mammalian
  misc_feature: The DNA sequence of the entire hygsa134 vector (with inserts)
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttctgcatgctgctgctgctgctgctgctgggcctgaggctacagctctccctgggcatcatcctagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgatgtgatcctaggtggaggccgaaagtacatgtttcgcatgggaaccccagaccctgagtacccagatgactacagccaaggtgggaccaggctggacgggaagaatctggtgcaggaatggctcggcgaacgccagggtgcccggtacgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcagacacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccttcatcgggctggccgctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttggaagatcctagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 12 - DNA, 440 bp, Unknown
  Mammalian
  misc_feature: CAPP134 cDNA insert in hygsa134 vector
agttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagc

SEQ ID NO: 13 - protein, 770 aa, Unknown
  Mammalian
  MISC_FEATURE: APPswe
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNLDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 14 - protein, 467 aa, Unknown
  Mammalian
  MISC_FEATURE: Presenilin 1 (PS1) amino acid sequence (homo sapiens)
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 15 - protein, 467 aa, Unknown
  Mammalian
  MISC_FEATURE: PS1M146V : A single mutation at codon 146 co-segregates with the disease in members of  early-onset Alzheimer′s disease families. A base pair change from the normal sequence predicts M to V amino acid substitution at codon 146.
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVVTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 16 - protein, 83 aa, Unknown
  Mammalian
  MISC_FEATURE: 8 kDa CAPPalpha (C83)
LVFFAEDVGSNKGAIIGLMVGGVVIATVIVITLVMLKKKQYTSIHHGVVEVDAAVTPEERHLSKMQQNGYENPTYKFFEQMQN

SEQ ID NO: 17 - DNA, 4154 bp, Unknown
  Mammalian
  misc_feature: SREBP-1a; SREBP-1 (whole mRNA sequence from Gene Bank Accession No. U00968, GI:409404)
taacgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcgccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttata

SEQ ID NO: 18 - protein, 1123 aa, Unknown
  Mammalian
  MISC_FEATURE: SREBP-1c amino acid sequence; identical to SREBP-1a (SEQ ID NO: 24) except for amino acid residues 1-6 of SREBP-1c (from SREBP-1c specific exon) differ from residues 1-30 of SREBP-1a (SEQ ID NO: 24)
MDCTFEDMLQLINNQDSDFPGLFDPPYAGSGAGGTDPASPDTSSPGSLSPPPATLSSSLEAFLSGPQAAPSPLSPPQPAPTPLKMYPSMPAFSPGPGIKEESVPLSILQTPTPQPLPGALLPQSFPAPAPPQFSSTPVLGYPSPPGGFSTGSPPGNTQQPLPGLPLASPPGVPPVSLHTQVQSVVPQQLLTVTAAPTAAPVTTTVTSQIQQVPVLLQPHFIKADSLLLTAMKTDGATVKAAGLSPLVSGTTVQTGPLPTLVSGGTILATVPLVVDAEKLPINRLAAGSKAPASAQSRGEKRTAHNAIEKRYRSSINDKIIELKDLVVGTEAKLNKSAVLRKAIDYIRFLQHSNQKLKQENLSLRTAVHKSKSLKDLVSACGSGGNTDVLMEGVKTEVEDTLTPPPSDAGSPFQSSPLSLGSRGSGSGGSGSDSEPDSPVFEDSKAKPEQRPSLHSRGMLDRSRLALCTLVFLCLSCNPLASLLGARGLPSPSDTTSVYHSPGRNVLGTESRDGPGWAQWLLPPVVWLLNGLLVLVSLVLLFVYGEPVTRPHSGPAVYFWRHRKQADLDLARGDFAQAAQQLWLALRALGRPLPTSHLDLACSLLWNLIRHLLQRLWVGRWLAGRAGGLQQDCALRVDASASARDAALVYHKLHQLHTMGKHTGGHLTATNLALSALNLAECAGDAVSVATLAEIYVAAALRVKTSLPRALHFLTRFFLSSARQACLAQSGSVPPAMQWLCHPVGHRFFVDGDWSVLSTPWESLYSLAGNPVDPLAQVTQLFREHLLERALNCVTQPNPSPGSADGDKEFSDALGYLQLLNSCSDAAGAPAYSFSISSSMATTTGVDPVAKWWASLTAVVIHWLRRDEEAAERLCPLVEHLPRVLQESERPLPRAALHSFKAARALLGCAKAESGPASLTICEKASGYLQDSLATTPASSSIDKAVQLFLCDLLLVVRTSLWRQQQPPAPAPAAQGASSRPQASALELRGFQRDLSSLRRLAQSFRPAMRRVFLHEATARLMAGASPTRTHQLLDRSLRRRAGPGGKGGAVAELEPRPTRREHAEALLLASCYLPPGFLSAPGQRVGMLAEAARTLEKLGDRRLLHDCQQMLMRLGGGTTVTSS

SEQ ID NO: 19 - DNA, 4249 bp, Unknown
  Mammalian
  misc_feature: SREBP-2; whole mRNA sequence from Genbank Accession No. U02031, GI:451329; CDS 118...3543; for cloning in pcDNA3.1, see SEQ ID NOs. 22 and 23
  misc_feature: SREBP-2; whole mRNA sequence from Genbank Accession No. U02031, GI:451329; CDS 118...3543
ccgtcggtgaggcggtgccgggcgggggttgtcgggtgtcatgggcggtggcgacggcaccgcccccgcgtctccctgagcgggacggcagggggggcttctgcgctgagccgggcgatggacgacagcggcgagctgggtggtctggagaccatggagaccctcacggagctgggcgacgagctgaccctgggagacatcgacgagatgctgcaatttgtcagtaatcaagtgggagagttccctgacttgttttcagaacagctgtgtagctcctttcctggcagtggtggtagtggtagcagcagcggcagcagtggcagcagcagcagcagcagcaatggcaggggcagcagcagcggagctgtggacccttcagtgcaacggtcattcacccaggtcacattaccttccttctctccctcggcggcctccccacaggctccaactctgcaagtcaaggtttctcccacctcagttcccaccacacccagggcaactcctattcttcagccccgcccccagccccagcctcaacctcaaactcagctgcaacaacagacggtaatgatcacgccaacattcagcaccactccgcagacgaggatcatccagcagcctttgatataccagaatgcagctactagctttcaagtccttcagcctcaagtccaaagcctggtgacatcctcccaggtacagccggtcaccattcagcagcaggtgcagacagtacaggcccagcgggtgctgacacaaacggccaatggcacgctgcagacccttgccccggctacggtgcagacagttgctgcgccacaggtgcagcaggtcccggtcctggtccagcctcagatcatcaagacagattcccttgttttgaccacactgaagacagatggcagccctgttatggctgcggtccagaacccggccctcaccgccctcaccacccctatccagacggctgcccttcaagtaccaaccctggtgggcagcagtgggaccattctgaccacaatgcctgtaatgatggggcaagagaaagtgcccattaagcaggtacctgggggagtcaagcagcttgagccccccaaagaaggagaaaggcggacaacccataatatcattgagaaacgatatcgctcctccatcaatgacaaaatcatcgaattgaaagacctggtcatggggacagacgccaagatgcacaagtctggcgttctgaggaaggccattgattacatcaaatacttgcagcaggtcaatcataaactgcgccaggagaacatggtgctgaagctggcaaatcaaaagaacaagcttctaaagggcatcgacctaggcagtctggtggacaatgaggtggacctgaagatcgaggactttaatcagaatgtccttctgatgtcccccccagcctctgactcagggtcccaggctggcttctctccctactccattgactctgagccaggaagccctctattggatgatgcaaaggtcaaagatgagccagactctcctcctgtggcgctgggcatggtagaccgctcacggattcttctgtgtgtcctcaccttcctgtgcctctcctttaaccccctgacttccctgctgcagtggggaggggcccacgactctgaccagcacccacactcaggctctggccgcagtgtcctgtcattcgagtcaggttctgggggctggtttgactggatgatgcctactcttctcttatggctggtaaatggtgtgattgtcctgagcgtctttgtgaagctgctggttcatggggagccagtgatccggccacactcgcgctcctcggtcaccttctggaggcaccggaaacaggcagatctggatctcgccagaggagattttgcagctgctgccgccaacctacaaacctgcctggcagttttgggccgggcactgcccacctcccgcctggacctggcctgcagcctctcctggaacgtgatccgctacagcctgcagaagctacgcctggtgcgctggctgctcaagaaagtcttccagtgccggcgggccacgccagccactgaggcaggctttgaagacgaagctaagaccagcgcccgggatgcggctctggcctatcaccggctgcaccagctgcacatcacagggaagcttcctgcaggatccgcctgttccgatgtacacatggcgttgtgtgccgtgaacctggctgaatgtgcagaggagaagatcccaccgagcacactggttgagatccatctgactgctgccatggggctcaagacccggtgtggaggcaagctgggcttcctggccagctacttcctcagccgagcccagagcctgtgtggccccgagcacagtgctgttcctgactccctgcgctggctctgccaccccctgggccagaagtttttcatggagcggagctggtctgtgaagtcagctgccaaggagagtctatactgtgcccagaggaacccagctgaccccattgcgcaggtccaccaggccttctgcaagaacctgctggagcgagctatagagtccttggtgaaacctcaggccaagaagaaggctggagaccaggaagaagagagctgtgaattctccagtgctctggagtacttgaaattacttcattcttttgtggactctgtgggggttatgagccccccactctccaggagctccgtgctcaagtccgccctgggtccagacatcatctgtcggtggtggacgtctgcaatcactgtggccatcagctggctccagggagacgatgcagctgtgcgctctcattttaccaaagtggaacgcatccccaaggccctggaagtgacagagagccccctggtgaaggccatcttccatgcctgcagagccatgcatgcctcactccctgggaaagcagatgggcagcagagttccttctgccattgcgagagggccagtggccacctatggagcagcctcaacgtcagtgggggcacctctgaccctgccctcaaccacgtggtccagctgctcacctgtgacctgctactgtcgctacggacagcgctctggcaaaaacaggccagtgccagccaggctgtgggggagacctaccacgcgtcaggcgctgaactggcgggcttccaacgggacctgggcagcctgcgcaggctggcacacagcttccgcccagcataccgcaaggtgttcctgcatgaagccaccgtgcgcctgatggcaggaggcagccccacccgcacccaccagctgctggaacacagcctgcggcggcgcaccacgcagagcaccaagcacggagaggtggatgcctggcccggccagcgagagcgggccaccgccatcctgctggcctgccgccacctgcccctctccttcctctcctccccgggccagcgggcagtgctgctggccgaagctgcccgcaccctggagaaggtgggcgaccggcgctcctgcaacgactgccagcagatgattgttaagctgggtggtggcactgccattgccgcctcctgaccaccaggctcagcccacccctccacctctctctcgatttctctctctccccctcagcatcttcccgctgagagtggtggggaagagccttgtcttcttagctgtcacctgccgaggcttctgggccactcaggccagtgcacccctgggcagagccccttaaagctgctgtcactagatgcccatggtccagggcctggtgggcgtgagaggataggtggcagggcagaaactgggcagccctgacttgatagcagcagggggagctcccaagctgccaagcccctgcctccagccttcctgagtttctctctcctgaaccctactctctcctttttgcttcctcagtttttatcaggctttctctgggggacagcagtctctgagcaccagggagcagttgccctcaggcctgtgcccagcatgccctcccctttttatacgaatgttttctaccagtgtgcttgggtttgccatgatgcgaggctgagttgctgtagcgtcttgattctctccctgggtctgcgttccctcccctgggcctgactgagcctgctcattgtttttccctttattacacaggacagccaggggaggaggggggcccagccctgggaggctggtgggaggcagggggcaggcctgcggatgcatgaaataatgttggcattattttttaattttttaaaaaataaatggtatctta

SEQ ID NO: 20 - DNA, 28 bp, Unknown
  Mammalian
  misc_feature: SREBP-1 forward primer; BamHI site at nucleotides 5-10; target sequence at nucleotides 13-28
gagaggatccaacagggcaggacacgaa

SEQ ID NO: 21 - DNA, 28 bp, Unknown
  Mammalian
  misc_feature: SREBP-1 reverse primer; EcoRI site at nucleotides 5-10; target sequence at nucleotides 13-28
gagagaattcggctgctgccaagggaca

SEQ ID NO: 22 - DNA, 29 bp, Unknown
  Mammalian
  misc_feature: SREBP-2 forward primer; BamHI site at nucleotides 5-10; target sequence at nucleotides 13-29
gagaggatccaaggttgtcgggtgtcatg

SEQ ID NO: 23 - DNA, 31 bp, Unknown
  Mammalian
  misc_feature: SREBP-2 reverse primer; EcoRI site at nucleotides 5-10; target sequence at nucleotides 13-31
gagagaattcggctggctcatctttgacctt

SEQ ID NO: 24 - protein, 1147 aa, Unknown [sequence omitted]

SEQ ID NO: 25 - protein, 1141 aa, Unknown [sequence omitted]

SEQ ID NO: 26 - protein, 167 aa, Unknown
  Mammalian
  MISC_FEATURE: Human Leptin
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 27 - protein, 141 aa, Unknown
  Mammalian
  MISC_FEATURE (2)..(2): Wherein “Xaa” represents Gln or Glu
  MISC_FEATURE (17)..(17): Wherein “Xaa” represents Asn, Asp, or Gln
  MISC_FEATURE (22)..(22): Wherein “Xaa” represents Thr or Ala
  MISC_FEATURE (23)..(23): Wherein “Xaa” represents Gln, Glu, or absent
  MISC_FEATURE (29)..(29): Wherein “Xaa” represents Gln or Glu
  MISC_FEATURE (49)..(49): Wherein “Xaa” represents Ile, Leu, Met or methionine sulfoxide
  MISC_FEATURE (51)..(51): Wherein “Xaa” represents Gln or Glu
  MISC_FEATURE (57)..(57): Wherein “Xaa” represents Gln or Glu
  MISC_FEATURE (58)..(58): Wherein “Xaa” represents Gln or Glu
  MISC_FEATURE (63)..(63): Wherein “Xaa” represents Ile, Leu, Met, or methionine sulfoxide
  MISC_FEATURE (67)..(67): Wherein “Xaa” represents Asn, Asp, or Gln
  MISC_FEATURE (70)..(70): Wherein “Xaa” represents Gln or Glu
  MISC_FEATURE (73)..(73): Wherein “Xaa” represents Asn, Asp, or Gln
  MISC_FEATURE (77)..(77): Wherein “Xaa” represents Asn, Asp, or Gln
  MISC_FEATURE (95)..(95): Wherein “Xaa” represents Trp or Gln
  MISC_FEATURE (125)..(125): Wherein “Xaa” represents Gln or Glu
  MISC_FEATURE (129)..(129): Wherein “Xaa” represents Gln or Glu
  MISC_FEATURE (131)..(131): Wherein “Xaa” represents Ile, Leu, Met, or methionine sulfoxide
  MISC_FEATURE (133)..(133): Wherein “Xaa” represents Trp or Gln
  MISC_FEATURE (134)..(134): Wherein “Xaa” represents Gln or Glu
VXDDTKTLIKTIVTRIXDISHXXSVSSKXKVTGLDFIPGLHPILTLSKXDXTLAVYXXILTSXPSRXVIXISXDLEXLRDLLHVLAFSKSCHLPXASGLETLDSLGGVLEASGYSTEVVALSRLXGSLXDXLXXLDLSPGC

SEQ ID NO: 28 - protein, 135 aa, Unknown
  Mammalian
  MISC_FEATURE (11)..(11): Wherein “Xaa” represents Asn, Asp, or Gln
  MISC_FEATURE (16)..(16): Wherein “Xaa” represents Thr or Ala
  MISC_FEATURE (17)..(17): Wherein “Xaa” represents Gln, Glu or absence of an amino acid residue
  MISC_FEATURE (23)..(23): Wherein “Xaa” represents Gln or Glu
  MISC_FEATURE (43)..(43): Wherein “Xaa” represents Ile, Leu, Met, or methione sulfoxide
  MISC_FEATURE (45)..(45): Wherein “Xaa” represents Gln or Glu
  MISC_FEATURE (51)..(51): Wherein “Xaa” represents Gln or Glu
  MISC_FEATURE (52)..(52): Wherein “Xaa” represents Gln or Glu
  MISC_FEATURE (57)..(57): Wherein “Xaa” represents Ile, Leu, Met, or methione sulfoxide
  MISC_FEATURE (61)..(61): Wherein “Xaa” represents Asn, Asp, or Gln
  MISC_FEATURE (64)..(64): Wherein “Xaa” represents Gln or Glu
  MISC_FEATURE (67)..(67): Wherein “Xaa” represents Asn, Asp, or Gln
  MISC_FEATURE (71)..(71): Wherein “Xaa” represents Asn, Asp, or Gln
  MISC_FEATURE (89)..(89): Wherein “Xaa” represents Trp or Gln
  MISC_FEATURE (119)..(119): Wherein “Xaa” represents Gln or Glu
  MISC_FEATURE (123)..(123): Wherein “Xaa” represents Gln or Glu
  MISC_FEATURE (125)..(125): Wherein “Xaa” represents Ile, Leu, Met, or methionine sulfoxide
  MISC_FEATURE (127)..(127): Wherein “Xaa” represents Trp or Gln
  MISC_FEATURE (128)..(128): Wherein “Xaa” represents Gln or Glu
TLIKTIVTRIXDISHXXSVSSKXKVTGLDFIPGLHPILTLSKXDXTLAVYXXILTSXPSRXVIXISXDLEXLRDLLHVLAFSKSCHLPXASGLETLDSLGGVLEASGYSTEVVALSRLXGSLXDXLXXLDLSPGC

SEQ ID NO: 29 - protein, 131 aa, Unknown
  Mammalian
  MISC_FEATURE (7)..(7): Wherein “Xaa” represents Asn, Asp, or Gln
  MISC_FEATURE (12)..(12): Wherein “Xaa” represents Thr or Ala
  MISC_FEATURE (13)..(13): Wherein “Xaa” represents Gln, Glu, or absence of an amino acid residue
  MISC_FEATURE (19)..(19): Wherein “Xaa” represents Gln or Glu
  MISC_FEATURE (39)..(39): Wherein “Xaa” represents Ile, Leu, Met or methionine sulfoxide
  MISC_FEATURE (41)..(41): Wherein “Xaa” represents Gln or Glu
  MISC_FEATURE (47)..(47): Wherein “Xaa” represents Gln or Glu
  MISC_FEATURE (48)..(48): Wherein “Xaa” represents Gln or Glu
  MISC_FEATURE (53)..(53): Wherein “Xaa” represents Ile, Leu, Met, or methionine sulfoxide
  MISC_FEATURE (57)..(57): Wherein “Xaa” represents Asn, Asp, or Gln
  MISC_FEATURE (60)..(60): Wherein “Xaa” represents Gln or Glu
  MISC_FEATURE (63)..(63): Wherein “Xaa” represents Asn, Asp, or Gln
  MISC_FEATURE (67)..(67): Wherein “Xaa” represents Asn, Asp, Gln
  MISC_FEATURE (85)..(85): Wherein “Xaa” represents Trp or Gln
  MISC_FEATURE (115)..(115): Wherein “Xaa” represents Gln or Glu
  MISC_FEATURE (119)..(119): Wherein “Xaa” represents Gln or Glu
  MISC_FEATURE (121)..(121): Wherein “Xaa” represents Ile, Leu, Met, or methionine sulfoxide
  MISC_FEATURE (123)..(123): Wherein “Xaa” represents Trp or Gln
  MISC_FEATURE (124)..(124): Wherein “Xaa” represents Gln or Glu
TIVTRIXDISHXXSVSSKXKVTGLDFIPGLHPILTLSKXDXTLAVYXXILTSXPSRXVIXISXDLEXLRDLLHVLAFSKSCHLPXASGLETLDSLGGVLEASGYSTEVVALSRLXGSLXDXLXXLDLSPGC

SEQ ID NO: 30 - protein, 126 aa, Unknown
  Mammalian
  MISC_FEATURE (2)..(2): Wherein “Xaa” represents Asn, Asp, or Gln
  MISC_FEATURE (7)..(7): Wherein “Xaa” represents Thr or Ala
  MISC_FEATURE (8)..(8): Wherein “Xaa” represents Gln, Glu, or absence of an amino acid residue
  MISC_FEATURE (14)..(14): Wherein “Xaa” represents Gln or Glu
  MISC_FEATURE (34)..(34): Wherein “Xaa” represents Ile, Leu, Met, or methionine sulfoxide
  MISC_FEATURE (36)..(36): Wherein “Xaa” represents Gln or Glu
  MISC_FEATURE (42)..(42): Wherein “Xaa” represents Gln or Glu
  MISC_FEATURE (43)..(43): Wherein “Xaa” represents Gln or Glu
  MISC_FEATURE (48)..(48): Wherein “Xaa” represents Ile, Leu, Met, or methionine sulfoxide
  MISC_FEATURE (52)..(52): Wherein “Xaa” represents Asn, Asp, or Gln
  MISC_FEATURE (55)..(55): Wherein “Xaa” represents Gln or Glu
  MISC_FEATURE (58)..(58): Wherein “Xaa” represents Asn, Asp, or Gln
  MISC_FEATURE (62)..(62): Wherein “Xaa” represents Asn, Asp, or Gln
  MISC_FEATURE (80)..(80): Wherein “Xaa” represents Trp or Gln
  MISC_FEATURE (110)..(110): Wherein “Xaa” represents Gln or Glu
  MISC_FEATURE (114)..(114): Wherein “Xaa” represents Gln or Glu
  MISC_FEATURE (116)..(116): Wherein “Xaa” represents Ile, Leu, Met, or methionine sulfoxide
  MISC_FEATURE (118)..(118): Wherein “Xaa” represents Trp or Gln
  MISC_FEATURE (119)..(119): Wherein “Xaa” represents Gln or Glu
IXDISHXXSVSSKXKVTGLDFIPGLHPILTLSKXDXTLAVYXXILTSXPSRXVIXISXDLEXLRDLLHVLAFSKSCHLPXASGLETLDSLGGVLEASGYSTEVVALSRLXGSLXDXLXXLDLSPGC

SEQ ID NO: 31 - protein, 113 aa, Unknown
  Mammalian
  MISC_FEATURE (1)..(1): Wherein “Xaa” represents Gln or Glu
  MISC_FEATURE (21)..(21): Wherein “Xaa” represents Ile, Leu, Met, or methionine sulfoxide
  MISC_FEATURE (23)..(23): Wherein “Xaa” represents Gln or Glu
  MISC_FEATURE (29)..(29): Wherein “Xaa” represents Gln or Glu
  MISC_FEATURE (30)..(30): Wherein “Xaa” represents Gln or Glu
  MISC_FEATURE (35)..(35): Wherein “Xaa” represents Ile, Leu, Met, or methionine sulfoxide
  MISC_FEATURE (39)..(39): Wherein “Xaa” represents Asn, Asp, or Gln
  MISC_FEATURE (42)..(42): Wherein “Xaa” represents Gln or Glu
  MISC_FEATURE (45)..(45): Wherein “Xaa” represents Asn, Asp, or Gln
  MISC_FEATURE (49)..(49): Wherein “Xaa” represents Asn, Asp, or Gln
  MISC_FEATURE (67)..(67): Wherein “Xaa” represents Trp or Gln
  MISC_FEATURE (97)..(97): Wherein “Xaa” represents Gln or Glu
  MISC_FEATURE (101)..(101): Wherein “Xaa” represents Gln or Glu
  MISC_FEATURE (103)..(103): Wherein “Xaa” represents Ile, Leu, Met, or methionine sulfoxide
  MISC_FEATURE (105)..(105): Wherein “Xaa” represents Trp or Gln
  MISC_FEATURE (106)..(106): Wherein “Xaa” represents Gln or Glu
XKVTGLDFIPGLHPILTLSKXDXTLAVYXXILTSXPSRXVIXISXDLEXLRDLLHVLAFSKSCHLPXASGLETLDSLGGVLEASGYSTEVVALSRLXGSLXDXLXXLDLSPGC

SEQ ID NO: 32 - protein, 105 aa, Unknown
  Mammalian
  MISC_FEATURE (13)..(13): Wherein “Xaa” represents Ile, Leu, Met or methionine sulfoxide
  MISC_FEATURE (15)..(15): Wherein “Xaa” represents Gln or Glu
  MISC_FEATURE (21)..(21): Wherein “Xaa” represents Gln or Glu
  MISC_FEATURE (22)..(22): Wherein “Xaa” represents Gln or Glu
  MISC_FEATURE (27)..(27): Wherein “Xaa” represents Ile, Leu, Met, or methionine sulfoxide
  MISC_FEATURE (31)..(31): Wherein “Xaa” represents Asn, Asp, or Gln
  MISC_FEATURE (34)..(34): Wherein “Xaa” represents Gln or Glu
  MISC_FEATURE (37)..(37): Wherein “Xaa” represents Asn, Asp, or Gln
  MISC_FEATURE (41)..(41): Wherein “Xaa” represents Asn, Asp, or Gln
  MISC_FEATURE (59)..(59): Wherein “Xaa” represents Trp or Gln
  MISC_FEATURE (89)..(89): Wherein “Xaa” represents Gln or Glu
  MISC_FEATURE (93)..(93): Wherein “Xaa” represents Gln or Glu
  MISC_FEATURE (95)..(95): Wherein “Xaa” represents Ile, Leu, Met, or methionine sulfoxide
  MISC_FEATURE (97)..(97): Wherein “Xaa” represents Trp or Gln
  MISC_FEATURE (98)..(98): Wherein “Xaa” represents Gln or Glu
IPGLHPILTLSKXDXTLAVYXXILTSXPSRXVIXISXDLEXLRDLLHVLAFSKSCHLPXASGLETLDSLGGVLEASGYSTEVVALSRLXGSLXDXLXXLDLSPGC

SEQ ID NO: 33 - protein, 93 aa, Unknown
  Mammalian
  MISC_FEATURE (1)..(1): Wherein “Xaa” represents Ile, Leu, Met, or methionine sufloxide
  MISC_FEATURE (3)..(3): Wherein “Xaa” represents Gln or Glu
  MISC_FEATURE (9)..(9): Wherein “Xaa” represents Gln or Glu
  MISC_FEATURE (10)..(10): Wherein “Xaa” represents Gln or Glu
  MISC_FEATURE (15)..(15): Wherein “Xaa” represents Ile, Leu, Met, or methionine sulfoxide
  MISC_FEATURE (19)..(19): Wherein “Xaa” represents Asn, Asp, or Gln
  MISC_FEATURE (22)..(22): Wherein “Xaa” represents Gln or Glu
  MISC_FEATURE (25)..(25): Wherein “Xaa” represents Asn, Asp, or Gln
  MISC_FEATURE (29)..(29): Wherein “Xaa” represents Asn, Asp, or Gln
  MISC_FEATURE (47)..(47): Wherein “Xaa” represents Trp or Gln
  MISC_FEATURE (77)..(77): Wherein “Xaa” represents Gln or Glu
  MISC_FEATURE (81)..(81): Wherein “Xaa” represents Gln or Glu
  MISC_FEATURE (83)..(83): Wherein “Xaa” represents Ile, Leu, Met, or methionine sulfoxide
  MISC_FEATURE (85)..(85): Wherein “Xaa” represents Trp or Gln
  MISC_FEATURE (86)..(86): Wherein “Xaa” represents Gln or Glu
XDXTLAVYXXILTSXPSRXVIXISXDLEXLRDLLHVLAFSKSCHLPXASGLETLDSLGGVLEASGYSTEVVALSRLXGSLXDXLXXLDLSPGC

SEQ ID NO: 34 - protein, 93 aa, Unknown; identical to SEQ ID NO: 33
  Mammalian
  MISC_FEATURE (1)..(1): Wherein “Xaa” represents Ile, Leu, Met, or methionine sulfoxide
  MISC_FEATURE (3)..(3): Wherein “Xaa” represents Gln or Glu
  MISC_FEATURE (9)..(9): Wherein “Xaa” represents Gln or Glu
  MISC_FEATURE (10)..(10): Wherein “Xaa” represents Gln or Glu
  MISC_FEATURE (15)..(15): Wherein “Xaa” represents Ile, Leu, Met or methionine sulfoxide
  MISC_FEATURE (19)..(19): Wherein “Xaa” represents Asn, Asp, or Gln
  MISC_FEATURE (22)..(22): Wherein “Xaa” represents Gln or Glu
  MISC_FEATURE (25)..(25): Wherein “Xaa” represents Asn, Asp, or Gln
  MISC_FEATURE (29)..(29): Wherein “Xaa” represents Asn, Asp, or Gln
  MISC_FEATURE (47)..(47): Wherein “Xaa” represents Trp or Gln
  MISC_FEATURE (77)..(77): Wherein “Xaa” represents Gln or Glu
  MISC_FEATURE (81)..(81): Wherein “Xaa” represents Gln or Glu
  MISC_FEATURE (83)..(83): Wherein “Xaa” represents Ile, Leu, Met, or methionine sulfoxide
  MISC_FEATURE (85)..(85): Wherein “Xaa” represents Trp or Gln
  MISC_FEATURE (86)..(86): Wherein “Xaa” represents Gln or Glu

SEQ ID NO: 35 - protein, 75 aa, Unknown
  Mammalian
  MISC_FEATURE (1)..(1): Wherein “Xaa” represents Asn, Asp, or Gln
  MISC_FEATURE (4)..(4): Wherein “Xaa” represents Gln or Glu
  MISC_FEATURE (7)..(7): Wherein “Xaa” represents Asn, Asp, or Gln
  MISC_FEATURE (11)..(11): Wherein “Xaa” represents Asn, Asp, or Gln
  MISC_FEATURE (29)..(29): Wherein “Xaa” represents Trp or Gln
  MISC_FEATURE (59)..(59): Wherein “Xaa” represents Gln or Glu
  MISC_FEATURE (63)..(63): Wherein “Xaa” represents Gln or Glu
  MISC_FEATURE (65)..(65): Wherein “Xaa” represents Ile, Leu, Met, or methione sulfoxide
  MISC_FEATURE (67)..(67): Wherein “Xaa” represents Trp or Gln
  MISC_FEATURE (68)..(68): Wherein “Xaa” represents Gln or Glu
XVIXISXDLEXLRDLLHVLAFSKSCHLPXASGLETLDSLGGVLEASGYSTEVVALSRLXGSLXDXLXXLDLSPGC

SEQ ID NO: 36 - protein, 62 aa, Unknown
  Mammalian
  MISC_FEATURE (16)..(16): Wherein “Xaa′ represents Trp or Gln
  MISC_FEATURE (46)..(46): Wherein ”Xaa′ represents Gln or Glu
  MISC_FEATURE (50)..(50): Wherein “Xaa′ represents Gln or Glu
  MISC_FEATURE (52)..(52): Wherein ”Xaa′ represents Ile, Leu, Met, or methionine sulfoxide
  MISC_FEATURE (54)..(54): Wherein “Xaa′ represents Trp or Gln
  MISC_FEATURE (55)..(55): Wherein ”Xaa′ represents Gln or Glu
DLLHVLAFSKSCHLPXASGLETLDSLGGVLEASGYSTEVVALSRLXGSLXDXLXXLDLSPGC

SEQ ID NO: 37 - protein, 54 aa, Unknown
  Mammalian
  MISC_FEATURE (8)..(8): Wherein “Xaa” represents Trp or Gln
  MISC_FEATURE (38)..(38): Wherein “Xaa” represents Gln or Glu
  MISC_FEATURE (42)..(42): Wherein “Xaa” represents Gln or Glu
  MISC_FEATURE (44)..(44): Wherein “Xaa” represents Ile, Leu, Met, or methionine sulfoxide
  MISC_FEATURE (46)..(46): Wherein “Xaa” represents Trp or Gln
  MISC_FEATURE (47)..(47): Wherein “Xaa” represents Gln or Glu
SKSCHLPXASGLETLDSLGGVLEASGYSTEVVALSRLXGSLXDXLXXLDLSPGC

SEQ ID NO: 38 - protein, 74 aa, Unknown
  Mammalian
  MISC_FEATURE (3)..(3): Wherein “Xaa” represents Gln or Glu
  MISC_FEATURE (6)..(6): Wherein “Xaa” represents Asn, Asp, or Gln
  MISC_FEATURE (10)..(10): Wherein “Xaa” represents Asn, Asp, or Gln
  MISC_FEATURE (28)..(28): Wherein “Xaa” represents Trp or Gln
  MISC_FEATURE (58)..(58): Wherein “Xaa” represents Gln or Glu
  MISC_FEATURE (62)..(62): Wherein “Xaa” represents Gln or Glu
  MISC_FEATURE (64)..(64): Wherein “Xaa” represents Ile, Leu, Met, or methionine sulfoxide
  MISC_FEATURE (66)..(66): Wherein “Xaa” represents Trp or Gln
  MISC_FEATURE (67)..(67): Wherein “Xaa” represents Gln or Glu
VIXISXDLEXLRDLLHVLAFSKSCHLPXASGLETLDSLGGVLEASGYSTEVVALSRLXGSLXDXLXXLDLSPGC

SEQ ID NO: 39 - protein, 63 aa, Unknown
  Mammalian
  MISC_FEATURE (17)..(17): Wherein “Xaa” represents Trp or Gln
  MISC_FEATURE (47)..(47): Wherein “Xaa” represents Gln or Glu
  MISC_FEATURE (51)..(51): Wherein “Xaa” represents Gln or Glu
  MISC_FEATURE (53)..(53): Wherein “Xaa” represents Ile, Leu, Met, or methionine sulfoxide
  MISC_FEATURE (55)..(55): Wherein “Xaa” represents Trp or Gln
  MISC_FEATURE (56)..(56): Wherein “Xaa” represents Gln or Glu
RDLLHVLAFSKSCHLPXASGLETLDSLGGVLEASGYSTEVVALSRLXGSLXDXLXXLDLSPGC

SEQ ID NO: 40 - protein, 52 aa, Unknown
  Mammalian
  MISC_FEATURE (6)..(6): Wherein “Xaa” represents Trp or Gln
  MISC_FEATURE (36)..(36): Wherein “Xaa” represents Gln or Glu
  MISC_FEATURE (40)..(40): Wherein “Xaa” represents Gln or Glu
  MISC_FEATURE (42)..(42): Wherein “Xaa” represents Ile, Leu, Met, or methionine sulforxide
  MISC_FEATURE (44)..(44): Wherein “Xaa” represents Trp or Gln
  MISC_FEATURE (45)..(45): Wherein “Xaa” represents Gln or Glu
SCHLPXASGLETLDSLGGVLEASGYSTEVVALSRLXGSLXDXLXXLDLSPGC

SEQ ID NO: 41 - protein, 138 aa, Unknown
  Mammalian
  MISC_FEATURE (4)..(4): Wherein “Xaa” represents Gln or Glu
  MISC_FEATURE (7)..(7): Wherein “Xaa” represents Gln or Glu
  MISC_FEATURE (22)..(22): Wherein “Xaa” represents Asn, Asp, or Gln
  MISC_FEATURE (27)..(27): Wherein “Xaa” represents Thr or Ala
  MISC_FEATURE (28)..(28): Wherein “Xaa” represents Gln, Glu, or absence of an amino acid residue
  MISC_FEATURE (34)..(34): Wherein “Xaa” represents Gln or Glu
  MISC_FEATURE (54)..(54): Wherein “Xaa” represents Met, methionine sufloxide, Leu, Ile, Val, Ala, or Gly
  MISC_FEATURE (56)..(56): Wherein “Xaa” represents Gln or Glu
  MISC_FEATURE (62)..(62): Wherein “Xaa” represents Gln or Glu
  MISC_FEATURE (63)..(63): Wherein “Xaa” represents Gln or Glu
  MISC_FEATURE (68)..(68): Wherein “Xaa” represents Met, methione sufloxide, Leu, Ile, Val, Ala, or Gly
  MISC_FEATURE (72)..(72): Wherein “Xaa” represents Asn, Asp, or Gln
  MISC_FEATURE (75)..(75): Wherein “Xaa” represents Gln or Glu
  MISC_FEATURE (78)..(78): Wherein “Xaa” represents Asn, Asp, or Gln
  MISC_FEATURE (82)..(82): Wherein “Xaa” represents Asn, Asp, or Gln
  MISC_FEATURE (100)..(100): Wherein “Xaa” represents Gln, Trp, Tyr, Phe, Ile, Val, or Leu
  MISC_FEATURE (130)..(130): Wherein “Xaa” represents Gln or Glu
  MISC_FEATURE (134)..(134): Wherein “Xaa” represents Gln or Glu
  MISC_FEATURE (136)..(136): Wherein “Xaa” represents Met, methionine sulfoxide, Leu, Ile, Val, Ala, or Gly
  MISC_FEATURE (138)..(138): Wherein “Xaa” represents Trp or Gln
VPIXKVXDDTKTLIKTIVTRIXDISHXXSVSSKXKVTGLDFIPGLHPILTLSKXDXTLAVYXXILTSXPSRXVIXISXDLEXLRDLLHVLAFSKSCHLPXASGLETLDSLGGVLEASGYSTEVVALSRLXGSLXDXLX

SEQ ID NO: 42 - protein, 8 aa, Unknown
  Mammalian
  MISC_FEATURE (1)..(1): Wherein “Xaa” represents Gln or Glu
XLDLSPGC

SEQ ID NO: 43 - protein, 35 aa, Homo sapiens
VPIQKVQDDTKTLIKTIVTRINDISHTQSVSSKQK

SEQ ID NO: 44 - protein, 15 aa, Homo sapiens
YKVQDDTKTLIKTIV

SEQ ID NO: 45 - protein, 13 aa, Homo sapiens
NVIQISNDLENLR